(19) United States
(12) Patent Application Publication
(10) Pub. No.: US 2022/0307089 A1
(43) Pub. Date: Sep. 29, 2022

(54) PROGNOSTIC MARKERS OF METASTATIC CANCER

(71) Applicants: The Trustees of Columbia University in the City of New York, New York, NY (US); Rutgers, The State University of New Jersey, New Brunswick, NJ (US)

(72) Inventors: Cory ABATE-SHEN, New York, NY (US); Juan ARRIAGA, New York, NY (US); Antonina MITROFANOVA, Newark, NJ (US)

(21) Appl. No.: 17/696,550

(22) Filed: Mar. 16, 2022

Related U.S. Application Data
(63) Continuation of application No. PCT/US2020/052868, filed on Sep. 25, 2020.
(60) Provisional application No. 62/905,630, filed on Sep. 25, 2019.

(51) Int. Cl. C12Q 1/6886

(52) U.S. Cl. CPC C12Q 1/6886; C12Q 2600/118; C12Q 2600/158

(57) ABSTRACT

The present technology is directed to methods for diagnosing metastasis, or assessing risk of metastasis, in a subject having cancer. The present technology is also directed to methods for treating a subject having metastatic cancer or an increased risk of cancer metastasis; through the development of gene signatures with prognostic value for determining metastasis in human patients undergoing AR therapy. When compared to controls, the expression level of the genes is highly correlative with the presence of metastasis, as well as the risk of future cancer metastasis. The present technology is also directed to biomarkers that can identify and categorize the needs of different patients for less or more aggressive therapy to prevent or treat metastatic disease outcome.

### CROSS REFERENCE TO RELATED APPLICATIONS

This application is a Continuation of and claims priority to International Application Serial No. PCT/US20/52868, filed on Sep. 25, 2020, which claims the benefit of and priority to U.S. Provisional Application No. 62/905,630, filed on Sep. 25, 2019, both entitled “Prognostic Markers of Metastatic Cancer” the disclosures of which are hereby incorporated by reference in their entirety.

### STATEMENT REGARDING FEDERALLY SPONSORED RESEARCH OR DEVELOPMENT

This invention was made with government support under Grant Nos. CA183929-04 and CA173481-06 awarded by the National Institutes of Health (NIH) and National Cancer Institute (NCI). The government has certain rights in the invention.

### BACKGROUND

The present technology relates to biomarkers related to the diagnosis, treatment, and management of cancer. In such endeavors, the ability to provide individualized therapies tends to increase the likelihood of a positive outcome for a patient. One of the factors in successful management of cancer treatment is the ability for the clinician to categorize the level of aggressiveness of cancerous tissue—that is, whether it is indolent or non-metastatic, versus high-risk or metastatic—and accordingly tailor the therapies available for the cancerous tissue.

At present, diagnosis of prostate cancer is based on histological grading of biopsy tissues (Gleason scoring) and serum prostate specific antigen (PSA) levels that stratify patients broadly into low- and high-risk groups. However, these clinical parameters fail to sub-stratify within the same risk-group such that individualized therapy can be administered instead of the current practice of overtreatment of patients.

Metastatic prostate cancer is a leading cause of cancer-related death in men. Indeed, while locally-invasive prostate cancer is relatively indolent, having a 5-year survival of >90%, metastatic prostate cancer is often lethal with 5-year survival of <30%. Usually prostate cancer metastasis is clinically manifested at advanced disease stages particularly, although not exclusively, following androgen deprivation therapy (ADT), which leads to the emergence of castration-resistant prostate cancer (CRPC). Indeed, CRPC is often metastatic (mCRPC) and frequently accompanied by highly aggressive disease variants, including neuroendocrine phenotypes (NEPC).

The predominant site of prostate cancer metastasis is bone (>70% of cases), which is associated with significant morbidity and mortality. However, current treatments are neither curative, nor do they specifically or particularly target bone metastasis. An overwhelming majority of men with metastatic prostate cancer develop metastases to bone. Yet, until now it has proven elusive to develop high-efficiency models that develop bone metastasis in the context of the native tumor microenvironment and during the natural evolution of tumor progression in vivo.

In recent years, the clinical landscape for treatment of metastatic bone cancer has greatly expanded, including next generation androgen receptor axis targeting agents (such as Enzalutamide, Abiraterone, and Apalutamide), immunotherapeutics (such as Sipuleucel-T and PD-1 inhibitors), radionuclide therapy (such as radium-223), and therapies targeting other oncogenic and genomic pathways (such as poly adenosine diphosphate-ribose polymerase (PARP) inhibitors). However, despite improvements in overall survival, none of these agents are curative, either individually or in combination, and few specifically target bone metastases.

It has been established that cancer metastases, including bone metastases, arise as a consequence of complex processes involving both cell-intrinsic features of tumor cells and the physiological milieu of the host tumor microenvironment. Yet, one of the major challenges for studying bone metastasis has been the paucity of models that capture its natural evolution during tumor progression—that is, models that recapitulate cell-intrinsic features of tumor cells and the physiological milieu of the native tumor microenvironment as occurs in vivo. Understanding the intricacies of lethal prostate cancer poses specific challenges due to difficulties in accurate modeling of metastasis in vivo. Indeed, while in vivo models based on prostate cancer cells implanted in bone have provided some information on molecular processes of prostate tumor growth in bone, these models do not fully capture the metastatic processes as occur during tumor evolution. Moreover, while several GEMMs de novo bone metastasis have been described, these have relatively low penetrance, making their use for molecular or preclinical investigations challenging.

Therefore, a need exists for biomarkers that can identify and categorize the needs of different patients for less or more aggressive therapy to prevent or treat metastatic disease outcome, or as novel end-points in clinical trials for evaluating the therapeutic value of novel drugs or drug combinations.

### BRIEF SUMMARY

In certain embodiments, the present technology is directed to a method for diagnosing metastasis in a subject having cancer, or for assessing risk of metastasis in a subject having cancer, the method comprising:

(a) obtaining a sample from the subject;

(b) determining in the sample an expression level of one or more of genes ATAD2, AZIN1, CCNE2, ERCC6L, LMNB1, MAD2L1, MCM4, RACGAP1, RAD21, RAD51AP1, SRPK1, TAF2, TMPO, TOP2A, WDHD1, or WDR12;

(c) comparing the expression level obtained in step (b) with a reference level or with an expression level of the one or more genes in a control sample; and

(d) diagnosing that the subject has metastasis or an increased risk of metastasis, if the expression level of at least one gene obtained in step (b) increases by at least 10% compared to the reference level or its expression level in the control sample.

In other embodiments, the present technology is directed to a method for diagnosing metastasis in a subject having cancer, or for assessing risk of metastasis in a subject having cancer, the method comprising:

(a) obtaining a sample from the subject;

(b) determining in the sample an expression level of one or more of genes ATAD2, AZIN1, CCNE2, ERCC6L, LMNB1, MAD2L1, MCM4, RACGAP1, RAD21, RAD51AP1, SRPK1, TAF2, TMPO, TOP2A, WDHD1, WDR12, CSE1L, LRPPRC, DHX9, UBA2, G3BP1, HEATR1, MRPL3, DDX21, ARL6IP1, CHD1L, PAXIP1, ACTL6A, RRP15, NUP107, CENPN, DBF4, SLC25A5, RAN, CCT5, HNRNPAB, AFG3L2, TMEM97, WDR3, CDC23, NFYA, MSH2, MAPKAPK5, POLR3B, GART, C1QBP, ECT2, DSCC1, TRMT12, SLBP, UNG, TTK, KIF20A, TRIP13, or TIMELESS;

(c) comparing the expression level obtained in step (b) with a reference level or with an expression level of the one or more genes in a control sample; and

(d) diagnosing that the subject has metastasis or an increased risk of metastasis, if the expression level of at least one gene obtained in step (b) increases by at least 10% compared to the reference level or its expression level in the control sample.

In other embodiments, the present technology is directed to a method for treating a subject with metastatic cancer or an increased risk of cancer metastasis, the method comprising:

(a) obtaining a sample from the subject;

(b) determining in the sample an expression level of one or more of genes ATAD2, AZIN1, CCNE2, ERCC6L, LMNB1, MAD2L1, MCM4, RACGAP1, RAD21, RAD51AP1, SRPK1, TAF2, TMPO, TOP2A, WDHD1, or WDR12;

(c) comparing the expression level obtained in step (b) with a reference level or with an expression level of the one or more genes in a control sample; and

(d) treating the subject for metastatic cancer or an increased risk of cancer metastasis, if the expression level of at least one gene obtained in step (b) increases by at least 10% compared to the reference level or its expression level in the control sample.

In other embodiments, the present technology is directed to a method for treating a subject with metastatic cancer or an increased risk of cancer metastasis, the method comprising:

(a) obtaining a sample from the subject;

(b) determining in the sample an expression level of one or more of genes ATAD2, AZIN1, CCNE2, ERCC6L, LMNB1, MAD2L1, MCM4, RACGAP1, RAD21, RAD51AP1, SRPK1, TAF2, TMPO, TOP2A, WDHD1, WDR12, CSE1L, LRPPRC, DHX9, UBA2, G3BP1, HEATR1, MRPL3, DDX21, ARL6IP1, CHD1L, PAXIP1, ACTL6A, RRP15, NUP107, CENPN, DBF4, SLC25A5, RAN, CCT5, HNRNPAB, AFG3L2, TMEM97, WDR3, CDC23, NFYA, MSH2, MAPKAPK5, POLR3B, GART, C1QBP, ECT2, DSCC1, TRMT12, SLBP, UNG, TTK, KIF20A, TRIP13, or TIMELESS;

(c) comparing the expression level obtained in step (b) with a reference level or with an expression level of the one or more genes in a control sample; and

(d) treating the subject for metastatic cancer or an increased risk of cancer metastasis, if the expression level of at least one gene obtained in step (b) increases by at least 10% compared to the reference level or its expression level in the control sample.

In other embodiments, the present technology is directed to a kit comprising:

(a) means for quantifying an expression level of one or more genes selected from the group consisting of ATAD2, AZIN1, CCNE2, ERCC6L, LMNB1, MAD2L1, MCM4, RACGAP1, RAD21, RAD51AP1, SRPK1, TAF2, TMPO, TOP2A, WDHD1, WDR12, CSE1L, LRPPRC, DHX9, UBA2, G3BP1, HEATR1, MRPL3, DDX21, ARL6IP1, CHD1L, PAXIP1, ACTL6A, RRP15, NUP107, CENPN, DBF4, SLC25A5, RAN, CCT5, HNRNPAB, AFG3L2, TMEM97, WDR3, CDC23, NFYA, MSH2, MAPKAPK5, POLR3B, GART, C1QBP, ECT2, DSCC1, TRMT12, SLBP, UNG, TTK, KIF20A, TRIP13, and TIMELESS, in a sample from a subject;

(b) means for comparing the expression level with a reference level or with an expression level of the one or more genes in a control sample; and, optionally,

(c) means for determining a therapy for treating the subject.

### BRIEF DESCRIPTION OF THE DRAWINGS

FIG. 1: A mouse model of highly penetrant bone metastasis. FIG. 1A: Strategy. Delivery of tamoxifen to adult NPK-CAG^{YFP} mice (for Nkx3.1^{CreERT2/+}; Pten^{flox/flox}; Kras^{LSL-G12D/+}; R26R-CAG-^{LSL-EYFP/+}) results in activation of Nkx3.1^{CreERT2} allele leading to gene recombination in prostatic luminal cells to inactivation of Pten (Pten^{flox/flox}) and activation of Kras (Kras^{LSL-G12D/+}) together with lineage marking with R26R-CAG-^{LSL-EYFP/+}. Over time, tumors form which lead to metastasis in the bone as well as to lungs, livers and brain. Primary tumors and metastases were analyzed by bulk and single-cell RNA sequencing, as well as whole exome sequencing. FIGS. 1B-C: Histopathological analyses. B. Representative images of prostate tumors, lung metastases, and the indicated bone metastatic sites showing ex vivo fluorescence, histology (H&E), or immunostaining for YFP. Shown are representative images from 5 independent mice. Scale bars represent 0.1 cm for the epifluorescence and 50 μm for histological images (H&E and IHC). C: Representative H&E images (top) or confocal images (bottom) of bone metastases (spine). Shown is co-expression of YFP, with luminal cytokeratin (Ck8), basal cytokeratin (Ck5), androgen receptor (AR), and Ki67. Scale bars represent 50 μm. FIGS. 1D-F: Single-cell RNA sequencing. Uniform manifold approximation and projection (UMAP) visualization of single-cell RNA sequencing analysis of matched primary tumor and bone samples isolated from NPK-CAG^{YFP} mice. D: UMAP projection showing the sample of origin; black corresponds to primary tumor and dark grey to the bone sample. E: UMAP projection of unsupervised clustering; colors indicate distinct clusters. F: Scaled expression (DESeq2 normalized values) of YFP, AR, Ck8, and CD45 expression levels; p-values were calculated by two-sample two-tailed Welch t-test).

FIG. 2: Bone metastases have distinct sub-clonal origin. FIG. 2A: Whole exome sequencing of matched trios from 5 independent mice of primary tumor, bone, and lung metastases showing evolutionary trees constructed by analyses of somatic mutations (i.e., substitutions and indels). The length of the lines indicates the number of mutations in each branch, and the colors indicate the mutations unique to or shared in the clones; shown are the bootstrap-derived p-values for each case. FIG. 2B: Combined phylogeny tree based on consistent evolutionary patterns across all trees in A. Meta-analysis p-value was calculated using Fisher's method through combining bootstrap-derived p-values from individual trees.

FIG. 3: Molecular analyses of bone metastasis. FIGS. 3A-B: Bulk RNA sequencing. Principal component analysis (PCA) based on RNA sequencing of primary tumors, lung metastases and bone metastases. The circle indicates the separation of the bone metastases relative to primary tumor and lung metastases. FIG. 3B: Heatmap showing the top 100 genes that contribute to principal component 1 from FIG. 3A: Shown are row scaled expression values (color). See also Dataset 1. FIGS. 3C-D: Single-cell RNA sequencing. FIG. 3C: UMAP visualization showing the sample of origin for the two clusters corresponding to the primary tumor and the bone metastatic cells (see FIG. 1D); black corresponds to primary tumor and dark grey to the bone metastatic cluster. FIG. 3D: Enrichment of bone metastasis signature in the bone metastatic versus primary tumor cell clusters. The p-value was calculated by a two-sample two-tailed Welch t-test. FIGS. 3E-F. Gene Set Enrichment Analyses (GSEA). FIG. 3E. GSEA using the mouse NPK-CAG^{YFP} bone metastasis signature (from the bulk RNA sequencing analyses) to query the reference single-cell bone versus primary tumor samples also from NPK-CAG^{YFP} mice. FIG. 3F: GSEA using the human bone metastasis signature based on the Balk dataset (Table S2) to query the reference mouse bone metastasis gene signatures (from the NPK-CAG^{YFP} mice). NES (normalized enrichment score) and p-values were estimated using 1,000 gene permutations.

FIG. 4: MYC is up-regulated in prostate cancer metastasis and necessary for bone tumor growth. FIG. 4A. Cross species pathway-based GSEA following pathway enrichment analysis using the Hallmarks and C2 databases pathways. Shown is the enrichment of pathways from the Balk human bone metastasis signature with pathways from the mouse bone metastasis signature (from NPK-CAG^{YFP} mice). NES and p-values were estimated using 1,000 gene permutations. See also Dataset 3. FIG. 4B: Violin plot depicting the distribution of the NESs (y-axis) which represent MYC activity levels (based on single-sample GSEA) in primary tumors from TCGA (n=497) compared with metastases from SU2C (n=270). The p-value was estimated using two-sample one-tailed Welch t-test. FIG. 4C: Immunohistochemical analyses of MYC protein expression in bone metastases. Shown are representative images of MYC staining of 34 clinical samples of metastatic prostate cancer, including 12 bone metastases. Clinical data for the patient samples is provided in Table S3, and quantification of the staining in bone and other metastases is shown in the present disclosure. FIGS. 4D-I: Analyses of MYC silencing for tumor growth in bone. FIG. 4D: Strategy. PC3 cells engineered to express luciferase (Luc) and green fluorescent protein (GFP) (PC3-Luc-GFP cells) were infected with the control shRNA (shControl) or shRNA to silence MYC and then implanted into the tibia of NOD-SCID mouse hosts and tumor growth in bone was monitored by IVIS imaging. FIG. 4E: Western blot image showing total protein extracts prepared from cells that had been infected with the indicated shRNA to silence MYC (shMYC#1 or (shMYC#2), or with the control shRNA (shControl). Shown are the approximate molecular weights of molecular weight markers (kDa); Actin is a control for protein loading. The uncropped Western image is shown in the present disclosure as well. FIG. 4F: Immunostaining for MYC in tumors from cells that had been infected with shControl or shMYC#1. Scale bars represent 50 μm. FIG. 4G: Growth curves comparing PC3-Luc-GFP cells infected with shMYC#1 or shMYC#2 or shControl (n=10/group). P-value was estimated by two-way ANOVA with Sidak's multiple comparisons against shControl; **** denotes p-value<0.0001. FIG. 4H: Representative IVIS bioluminescence imaging from panel G. FIG. 4I: Representative images from the time of sacrifice of tibias implanted with the PC3-Luc-GFP cells infected with shMYC#1 or shMYC#2 or shControl. Shown are ex vivo imaging of YFP fluorescence, to visualize the tumor, and corresponding micro-computed tomography (CT) images, to show areas of osteolysis as is typical of PC3 tumors in bone. Also shown are representative H&E and immunostaining for YFP. Scale bars represent 0.1 cm for the fluorescent images and 50 μm for histological images (H&E and IHC).

FIG. 5. Myc silencing impairs bone metastasis in an allograft model. FIG. 5A: Strategy for allograft assay for bone metastasis. NPK bone cells that had been isolated from NPK-CAG^{YFP} mice were infected with the control shRNA (shControl) or shRNA to silence Myc and introduce in Nude mouse hosts via intracardiac injection to monitor metastasis in vivo. FIG. 5B: Western blot image showing total protein extracts prepared from cells that had been infected with the indicated shRNA to silence Myc (shMyc#1 or (shMyc#2), or with the control shRNA (shControl). Shown are the approximate molecular weights of molecular weight markers (kDa); Actin is a control for protein loading. The uncropped Western image is shown in Figure S8A. FIG. 5C: Representative ex vivo imaging of YFP fluorescence from the heart (injection site), lung, and the indicated bones from Nude mouse hosts following via intracardiac injection of NPK bone cells infected with shMyc#1 or shMyc#2 or shControl. FIG. 5D. Quantification of the number of metastases in bone or lung from NPK bone cells infected with shMyc#1 or shMyc#2 or shControl (as in panel C). The p-values were estimated based on One-way ANOVA with Dunnett's multiple comparisons against shControl; NS, not significant. FIG. 5E. Representative corresponding images of vertebrae showing ex vivo H&E and immunostaining for YFP of Myc. Scale bars represent 0.1 cm for the fluorescent images and 50 μm for histological images (H&E and IHC).

FIG. 6. A gene signature prognostic for time to metastasis in primary prostate tumors. FIG. 6A: Strategy for identification of the META-55 and META-16 gene signatures. In step 1, we performed genome-wide Spearman correlation analysis to MYC expression in the PROMOTE patient cohort (which includes 55 bone metastases), which identified 559 genes (PROMOTE-559) positively correlated with MYC (FDR p-value<0.0001, Spearman rank correlation coefficient rho plotted in the x-axis in FIG. 6B). In step 2, we performed GSEA using the PROMOTE-559 genes to query the mouse (NPK-CAG^{YFP}) and human (Balk) bone metastasis signatures (see Figure S9A, B). The leading edge genes from the mouse are projected on the y-axis and from the human on the z-axis in FIG. 6B. This identified 55 genes (highlighted in red in FIG. 6B) that are correlated to MYC expression, and upregulated in bone metastases vs primary tumors in both human and mouse bone metastasis signatures termed the META-55. In step 3, the 55 genes were ranked according to their ability to predict time-to-metastasis (i.e., metastasis-free) survival in TCGA patient cohort using Cox proportional hazards model and a Wald p-value<1×10⁻⁷ cutoff. This identified 16 genes most significantly associated with metastasis-free survival that were termed the META-16 signature herein. FIG. 6B: Visualization of the META-55 discovery from Steps 1-2 in A. The META-55 genes are indicated by shading (red), and the subset of the META-16 genes (from Step 3 in A) are shown by name. FIGS. 6C-D. UMAP projection of single-cell RNA sequencing depicting enrichment of the MYC pathway (in FIG. 6C) and the META-16 gene signature (in FIG. 6D). Scaled DESeq2 normalized values are depicted. Shown is the correlation between META-16 expression at the single-cell level with MYC pathway activity. The p-value was estimated using Spearman's rank correlation. FIG. 6E. Violin plot depicting the distribution of the NESs (y-axis) which reflect activity levels of META-16 in primary tumors from TCGA (n=497) compared with metastases from SU2C (n=270). The p-value was estimated using two-sample one-tailed Welch t-test. The p-value for the random model was p-value=0.036. FIG. 6G-K: Association of META-16 with time to metastasis. FIGS. 6G-H: Heatmaps of hierarchical consensus clustering used to define tumors with high (brown cluster) and low (green cluster) expression of the META-16 signature in Mayo (n=235) and JHMI (n=260) cohorts, as indicated (Table S2). Brown vertical bars on the second from top row represent patient cases that developed distant metastasis. FIG. 6I-J: Kaplan-Meier survival analyses comparing patients with the low and high expression of META-16 from panels FIG. 6G and FIG. 6H. The p-values were estimated using a log-rank test. FIG. 6K. Multivariable survival analysis of the META-16 gene signature in the JHMI and MAYO cohorts showing independent association with metastasis-free survival but not with prostate-cancer specific mortality (HR=hazard ratio, CI=confidence interval, p-values estimated from Cox proportional hazards model).

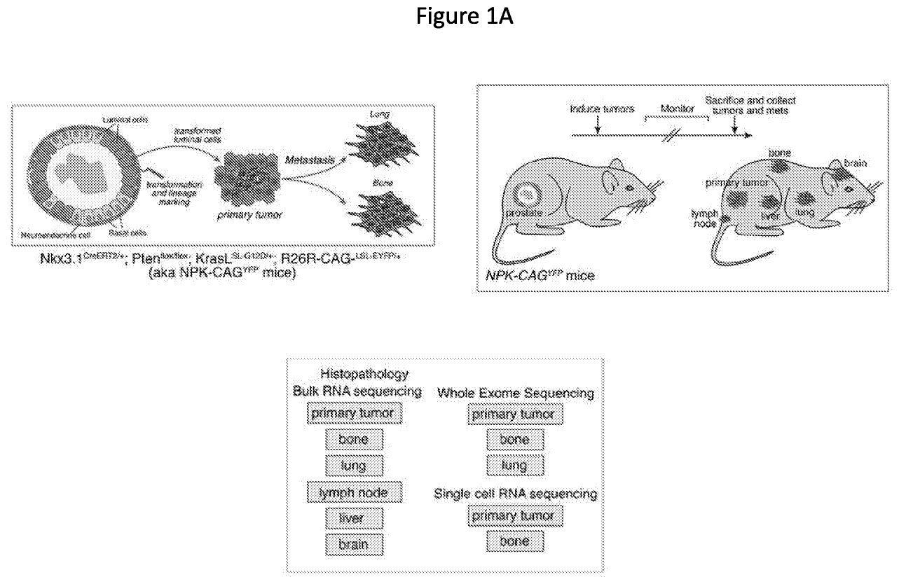

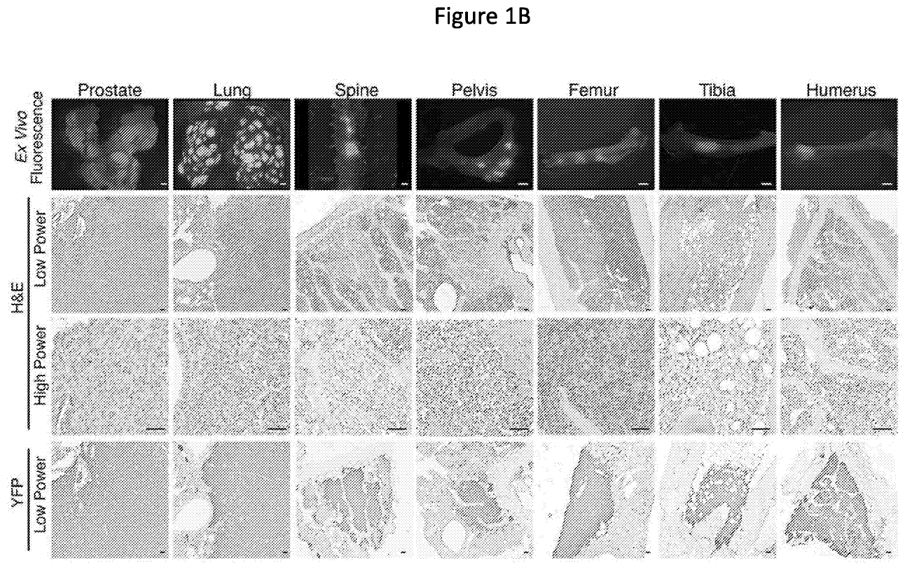

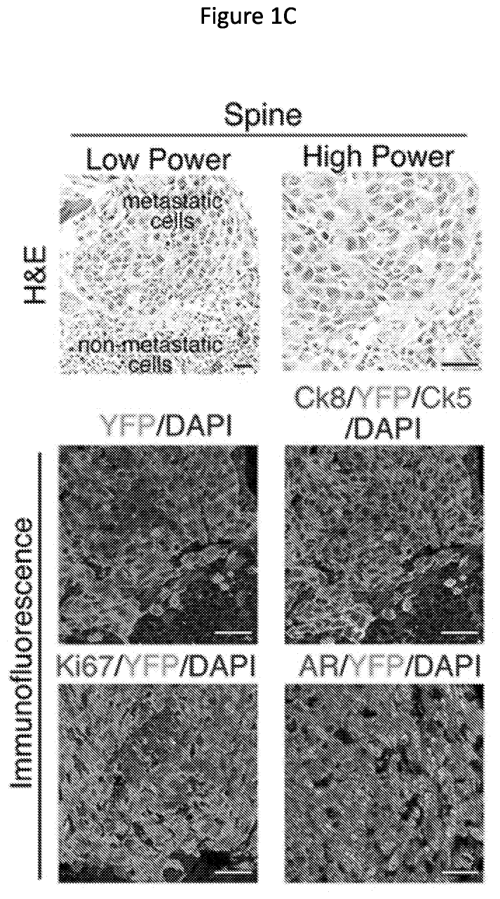

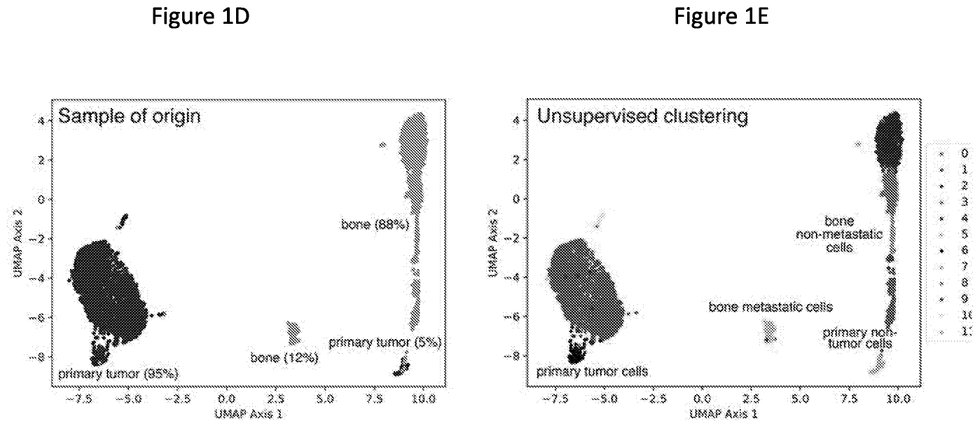

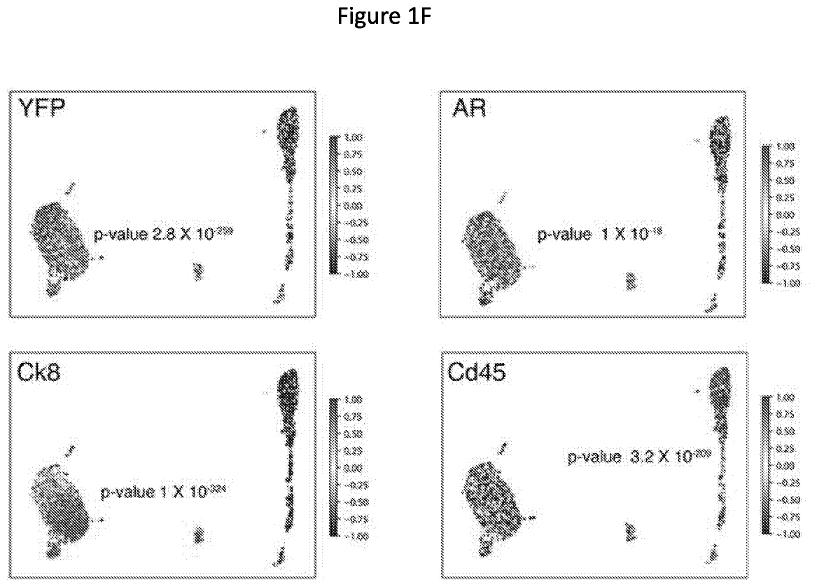

FIG. S1: Additional histological analyses of NPK-CAG^{YFP} prostate tumors (related to FIG. 1). FIG. S1A: Representative H&E sections of primary tumors in the DLP and AP lobes of NPK-CAG^{YFP} mice showing a mixture of high- and low-grade adenocarcinomas with frequent sarcomatoid differentiation and stromal components. No obvious histological differences were observed in tumors of mice without (left) or with (right) bone metastases. FIG. S1B: Representative images of prostate tumors and the indicated metastatic sites showing ex vivo fluorescence, histology (H&E), or immunostaining for androgen receptor (AR) or with the proliferation marker (Ki67). Shown are representative images from 5 independent mice. Scale bars represent 0.1 cm for the fluorescent images and 50 for histological images (H&E and IHC).

FIG. S2: Biological and molecular characteristics of NPK-CAG^{YFP} mice related to FIG. 1). FIGS. S2A-C. Comparison of NPK-CAG^{YFP} mice with (n=47) or without (n=59) bone metastasis. FIG. S2A: Overall survival; log-rank p-value, FIG. S2B: Bladder obstruction; p-value shown by two-sided Fisher's exact test. FIG. S2C: Tumor weight and metastatic load; p-value shown by two-tailed Mann-Whitney test. See also Table S1, FIG. S2D. Gene set enrichment analysis (GSEA) using a gene signature from a human osteoblast-induced, prostate cancer metastasis-specific dataset (34) to query the NPK-CAG^{YFP} bone metastasis reference signature. Normalized enrichment score (NES) and p-value were estimated with 1,000 gene permutations. FIGS. S2E-G. Comparison of intact (n=106) and castrated (n=22) NPK-CAG^{YFP} mice. FIG. S2E. Overall survival; log-rank p-value. FIG. S2F. Tumor weight and bone metastases; p-value shown by two-tailed Mann-Whitney test. See also Table S1. FIG. S2C. GSEA using the bone metastasis signature from castrated NPK-CAG^{YFP} mice to query the bone metastasis reference signature from the intact mice; NES and p-values were estimated using 1,000 gene permutations.

FIG. S3: RNA sequencing analyses of NPK-CAG^{YFP} prostate tumors (related to FIG. 1). FIG. S3A. Heatmap of the top 100 differentially expressed genes in primary prostate tumors from mice with (n=10) versus without (n=4) bone metastasis. Shown are row-scaled expression values (color). See also Dataset 1. FIGS. S3B-1, S3B-2, and S3B-3: GSEA of the selected significantly enriched pathways querying a signature that compares primary prostate tumors from mice with (n=10) versus without (n=4) bone metastasis. NESs and p-values were estimated with 1,000 gene permutations. See also Dataset 3.

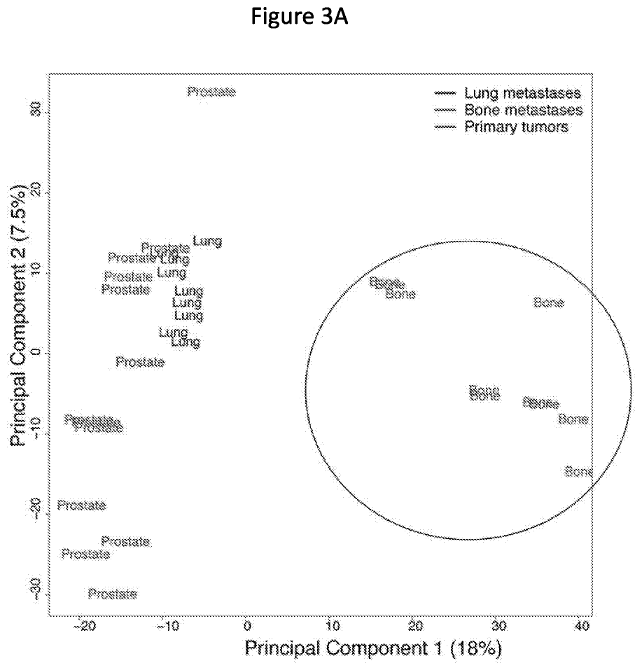

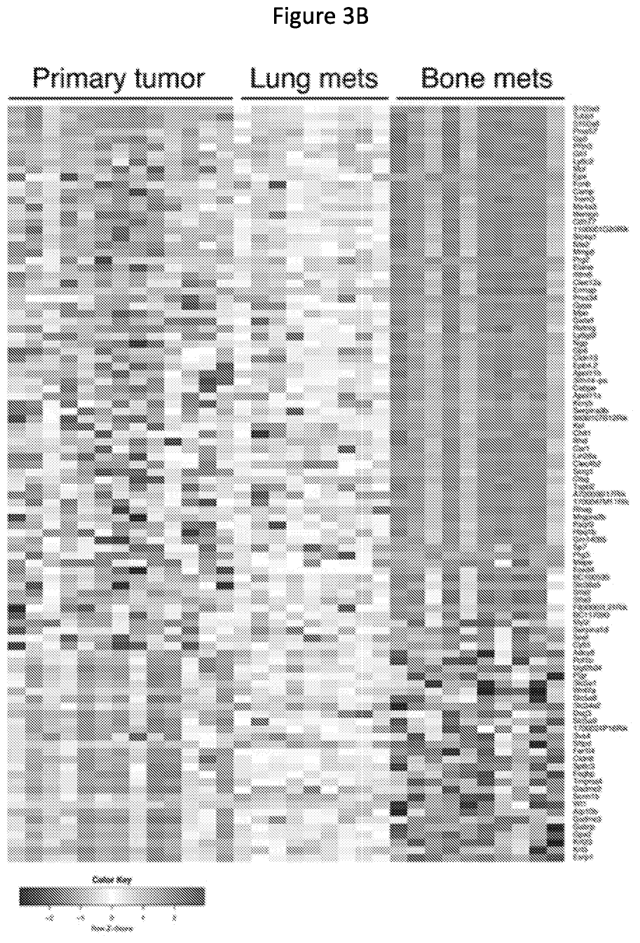

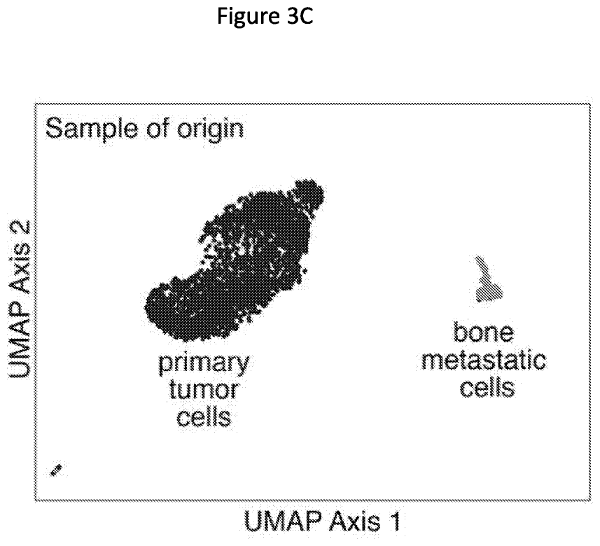

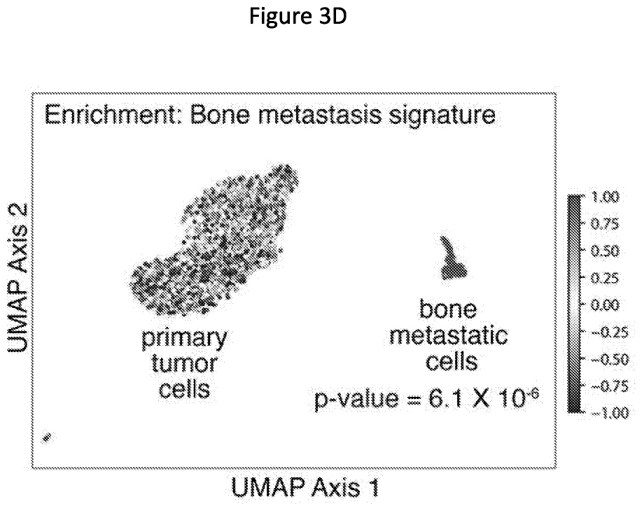

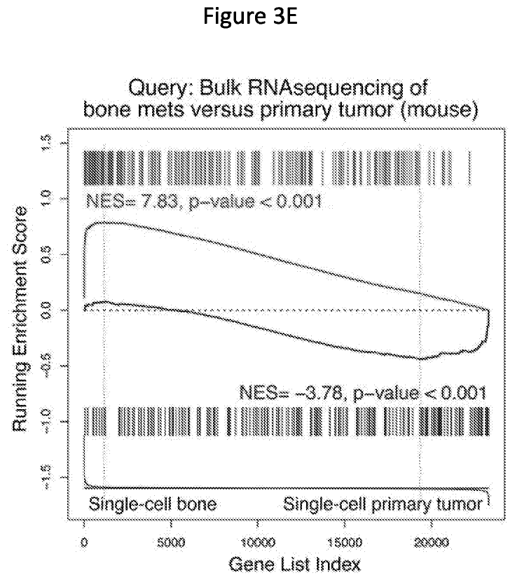

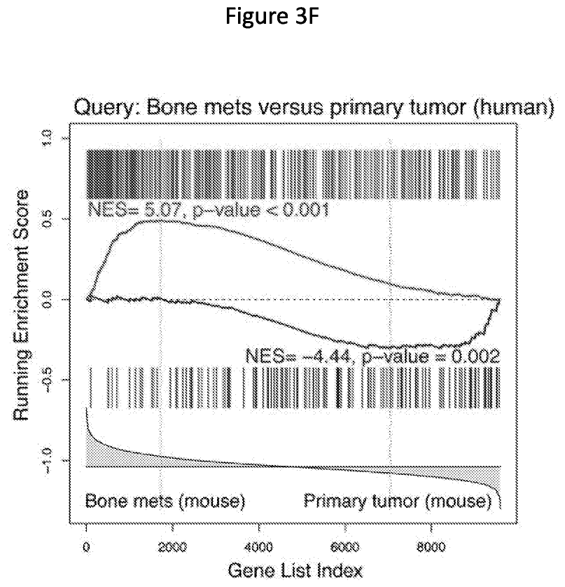

FIG. S4: RNA sequencing of PK-CAG^{YFP} prostate tumors and metastases (related to FIG. 3). FIG. S4A: Principal component analysis (PCA) based on RNA-sequencing analyses of primary tumors and the indicated metastatic sites in intact and castrated mice. The circle indicates the bone metastases, which cluster separately from the primary tumors and other metastases. FIG. S4B: Heatmap showing the top 100 genes that contribute to principal component 1 from (A). Cas, castrated. Shown are row-scaled expression values (color). See also Dataset 1.

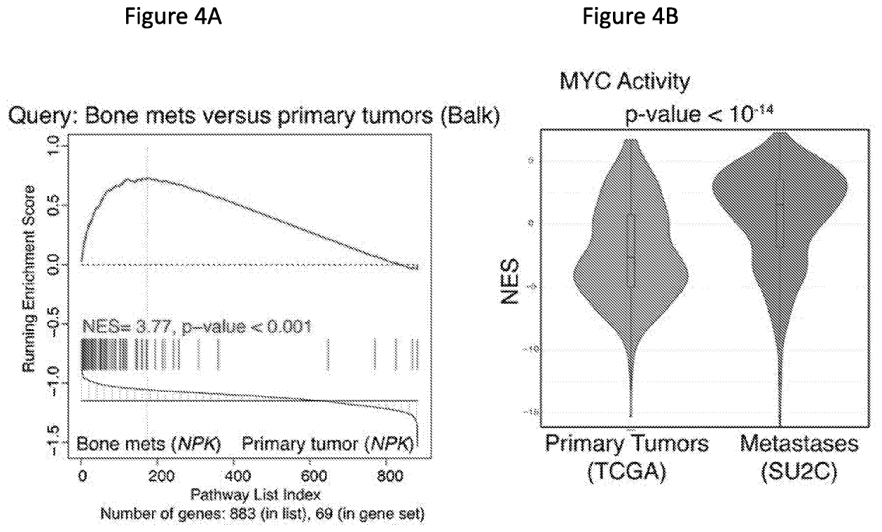

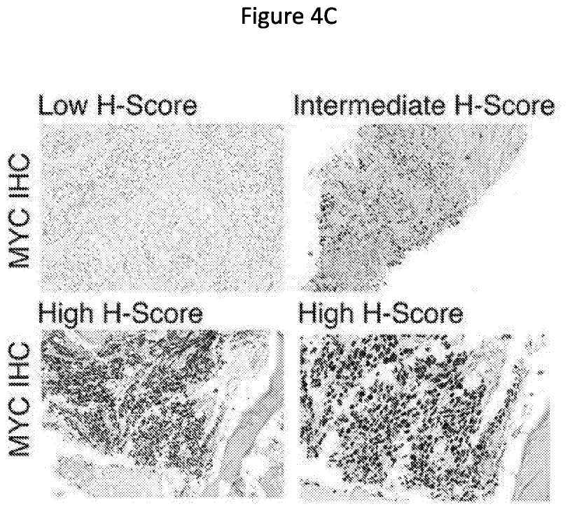

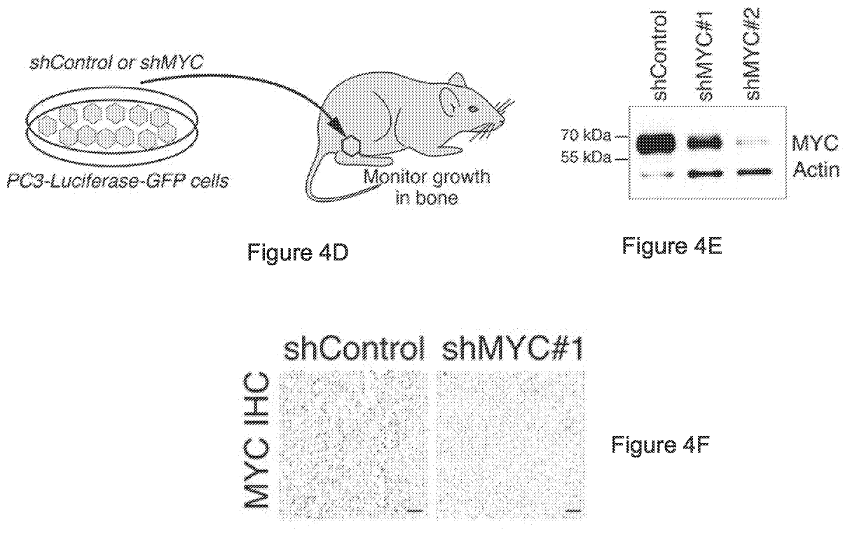

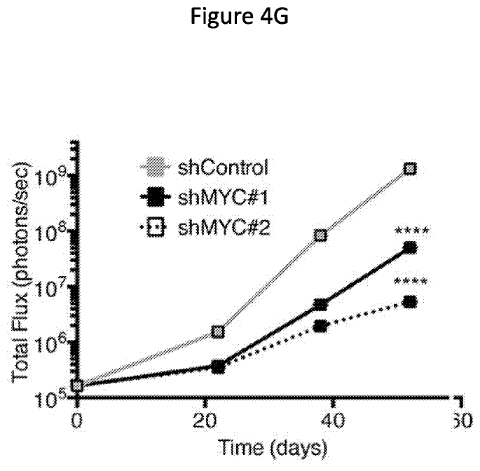

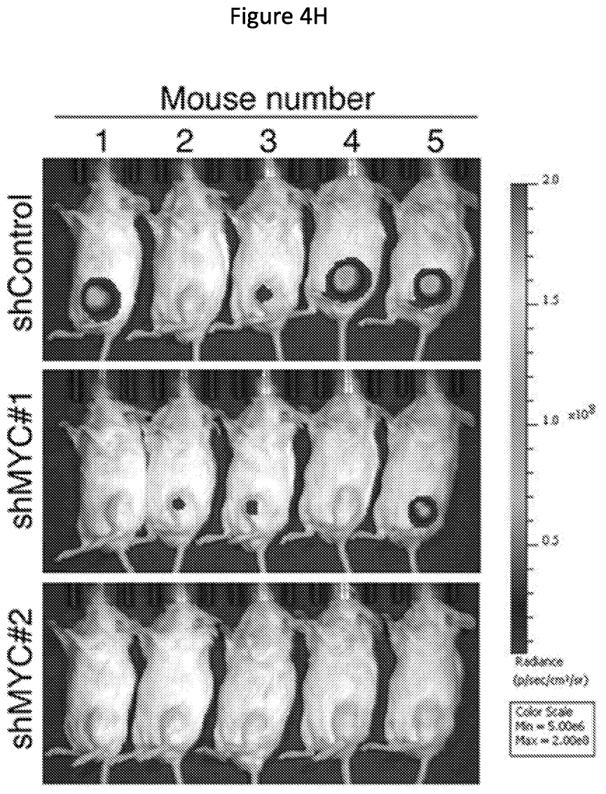

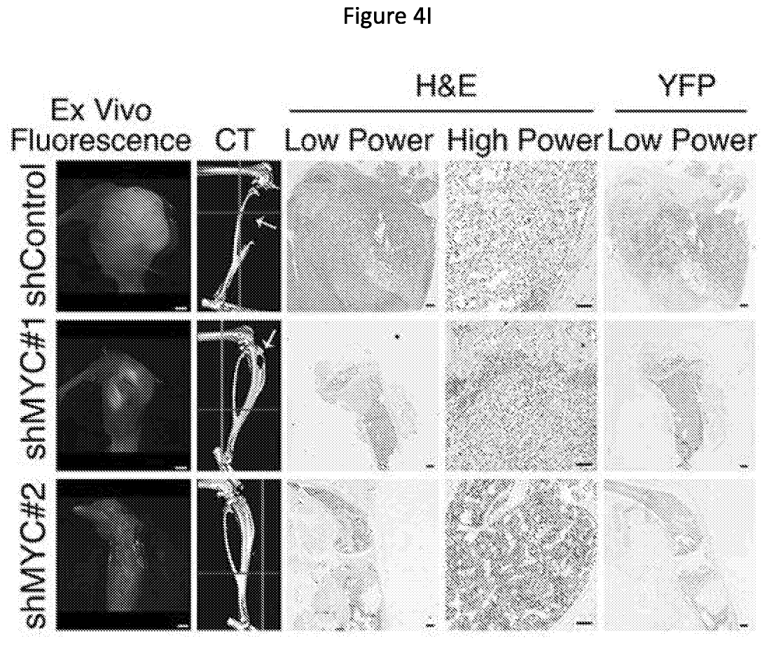

FIG. S5: Additional analyses of MYC activity in prostate tumors and metastases (related to FIG. 4). FIG. S5A: Cross species pathway-based GSEA following pathway enrichment analysis using the Hallmarks and C2 databases pathways. Shown is the enrichment of pathways from the FRCRC human bone metastasis with pathways from the mouse bone metastasis from NPK-CAG^{YFP} mice. NES and p-values were estimated using 1,000 gene permutations. See also Dataset 3. FIG. S5B: Stouffer integration of the leading-edge pathways from the G-SEA comparing mouse and the two human bone metastases signatures from FIG. S5A and FIG. 4A shows that MYC is the highest-ranked conserved pathways enriched in bone metastases versus primary tumors. The x-axis shows the Stouffer integrated NES, FIG. S5C-E: GSEA of bone metastasis signatures from NPK-CAG^{Y}″ mice (left), and the Balk (human, middle) and FRCRC (human, right) datasets showing enrichment of three independent MYC signatures.

FIG. S5C: “Hallmarks MYC” (human). FIG. S5D: “Dang MYC” (human). FIG. S5E. “Sabo Myc” (mouse). NES and p-values were estimated using 1,000 gene permutations. See also Dataset 3. FIG. S5F: Heatmap representation of single-sample GSEA enrichment of the MYC activity based on the Hallmarks MYC pathway in primary tumors from TCGA (n=497) and metastases from SU2C (n=270) cohorts (Table S2). Colors correspond to NES. FIG. S5G: Summary of H-scores from immunostaining analyses of MYC expression in human bone (n=12), liver (n=7) and lymph node (n=15) metastases from the JHH cohort (see Table S3). No difference was observed between the metastatic sites (two-tailed Mann-Whitney test).

FIG. S6: Additional analyses of MYC silencing in human PC3-Luc-GFP cells (related to FIG. 4). FIGS. S6A-B: In vitro analyses of PC3-Luc-GFP cells infected with the indicated shRNA to silence MYC (shMYC#1 or (shMYC#2), or with the control shRNA (shControl). FIG. S6A: Shown is the uncropped Western blot image highlighting the region shown in FIG. 4E by the red rectangle, and the approximate molecular weights of molecular weight markers (kDa). FIG. S6B: Colony formation analyses. Right, representative images of crystal violet-stained colonies. Left, quantification; p-values were estimated by one-way ANOVA with Dunnett's multiple comparisons against shControl. In vitro assays were repeated 3 times in triplicate; a representative experiment is shown. FIG. S6C-E: In vivo subcutaneous growth curves of PC3-Luc-GFP cells infected with the indicated shRNA to silence MYC (shMYC#1 or shMYC#2), or with the control shRNA (shControl). C. Growth curves comparing PC3-Luc-GFP cells infected with shControl or shMYC#1 (n=5/group). p-value was estimated by two-way ANOVA with Sidak's multiple comparisons against shControl. FIG. S6D: Photographs of the tumors at sacrifice. FIG. S6E: Representative IVIS bioluminescence imaging. In all panels, ** denotes p-value<0.01, *** denotes p-value<0.001 and **** denotes p-value<0.0001.

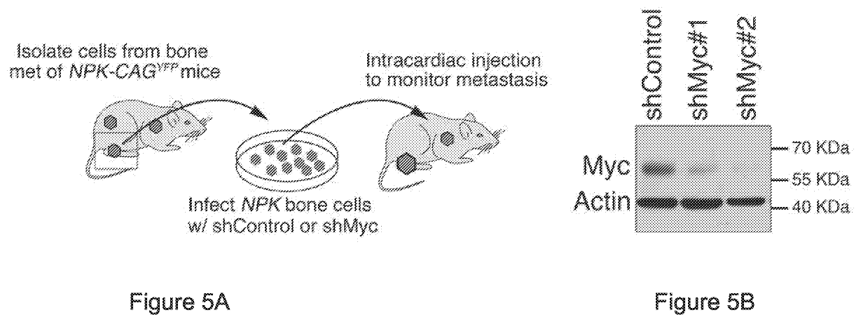

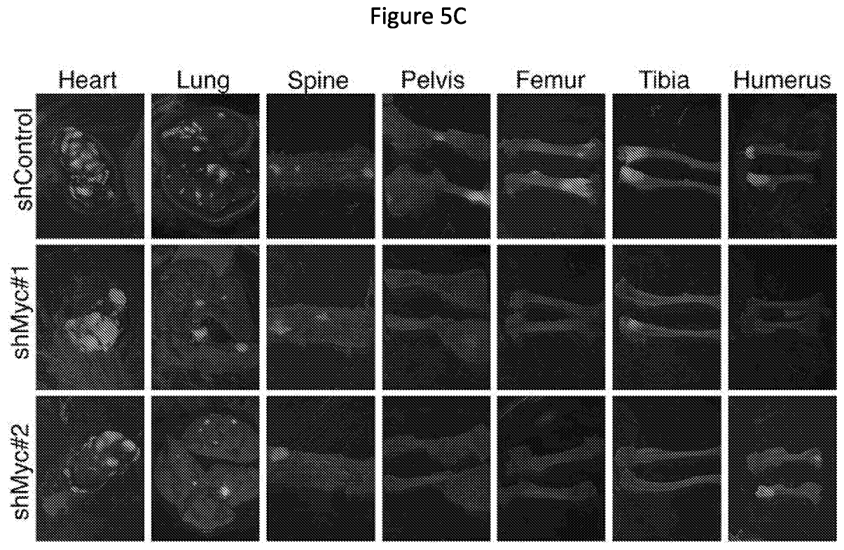

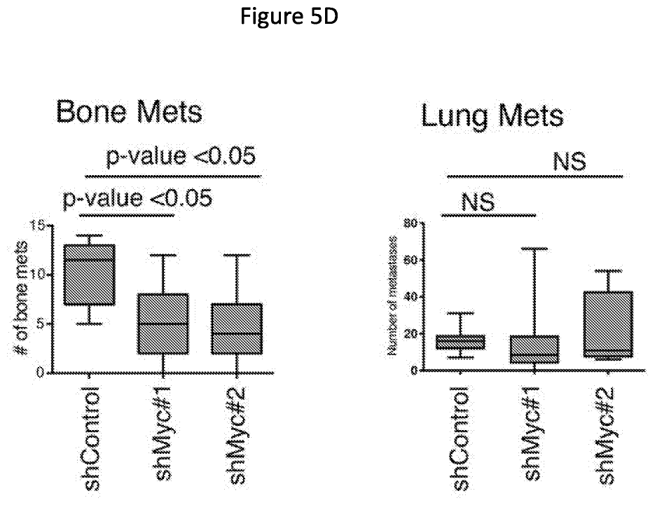

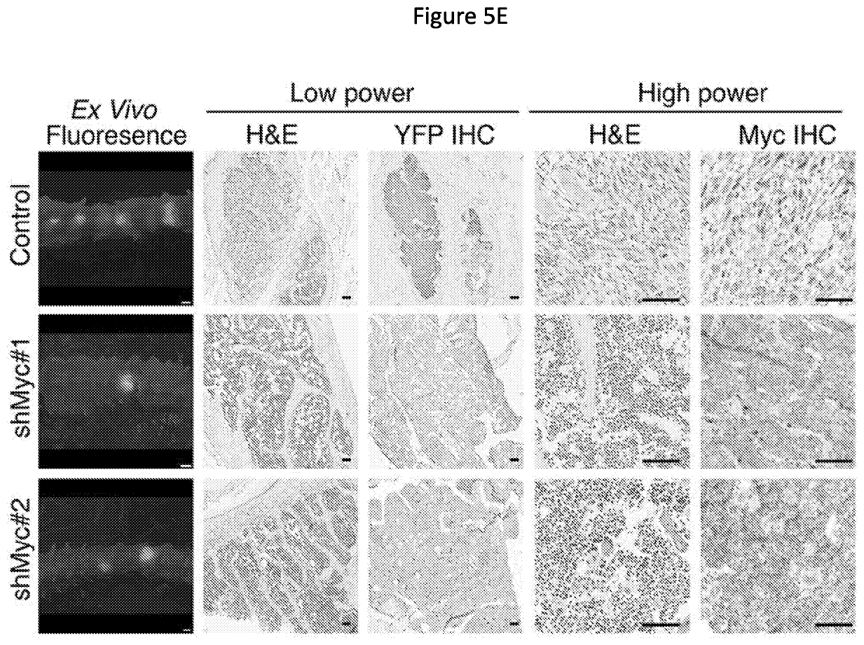

FIG. S7: Generation of an in vivo metastasis model based on NPK bone cells (related to FIG. 5). FIG. S7A: Strategy for generation of a bone metastasis allograft model of NPK-CAG^{YFP} mice. Bone metastases were isolated from NPK-CAG^{YFP} mice and cultured in vitro. Once established, the cells were passaged in Nude mouse hosts via intracardiac injection. A derivative cell line obtained from an ensuing bone metastasis, termed NPK bone cells, was used for the studies described herein. FIG. S7B. Comparison of lung and bone from Nude mouse hosts implanted via intracardiac injection with cells derived from primary tumors of non-metastatic NP-CAG^{YFP} mice from known protocols, or with the NPK bone cells. Shown are representative ex vivo fluorescence or H&E images. Scale bars represent 0.1 cm for the fluorescent images and 50 μm for histological images (H&E).

FIG. S8: Additional analyses of Myc silencing in mouse NPK bone cells (related to FIG. 5). In vitro analyses of NPK bone cells infected with the indicated shRNA to silence Myc (shMyc#1 or (shMyc#2), or with the control shRNA (shControl). FIG. S8A: Shown is the uncropped Western blot image highlighting the region shown in FIG. 5B by the red rectangle, and the approximate molecular weights of molecular weight markers (kDa). FIG. S8B: Colony formation analyses. Right, representative images of crystal violet-stained colonies. Left, quantification. As indicated, ** p-value<0.01; p-values were estimated by one-way ANOVA with Dunnett's multiple comparisons against shControl. In vitro assays were repeated 3 times in triplicate; a representative experiment is shown.

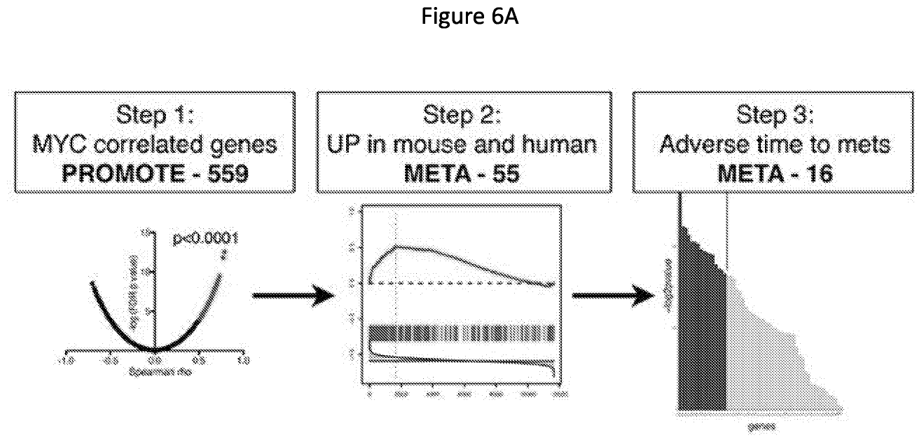

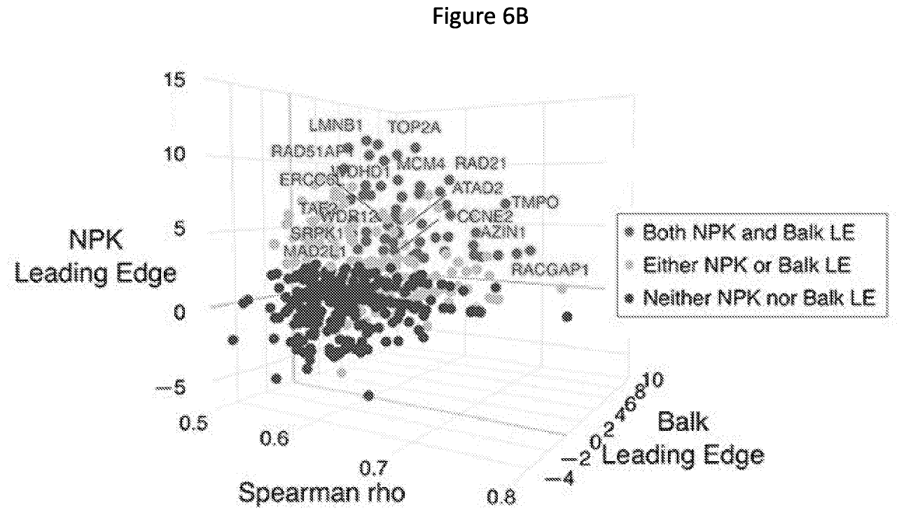

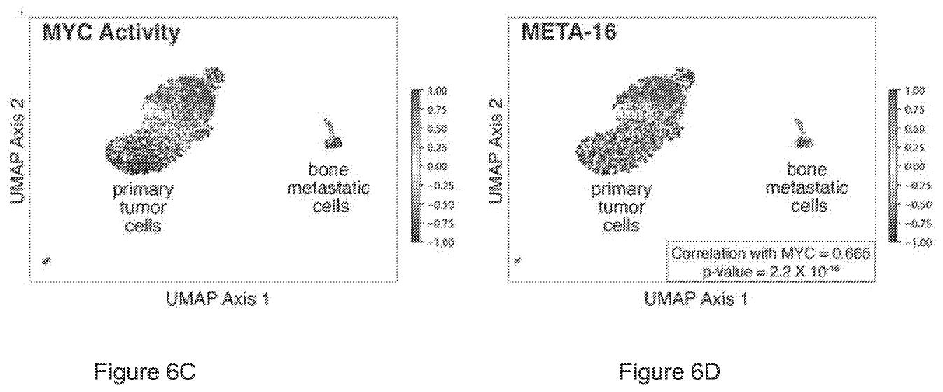

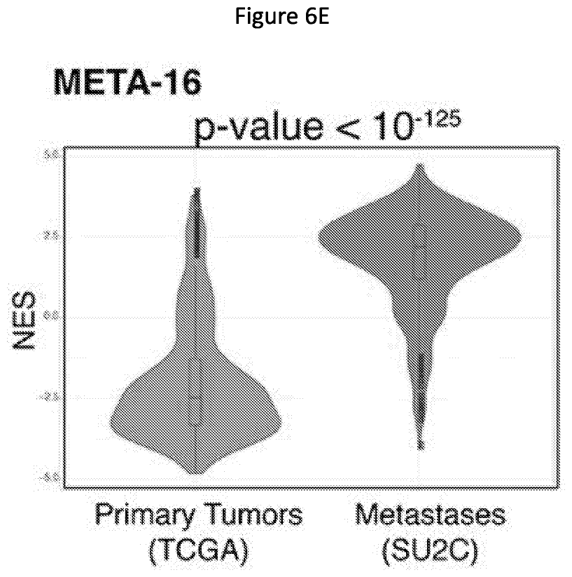

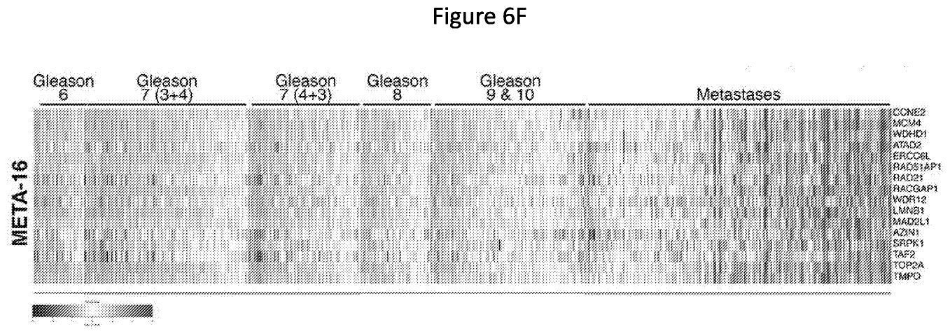

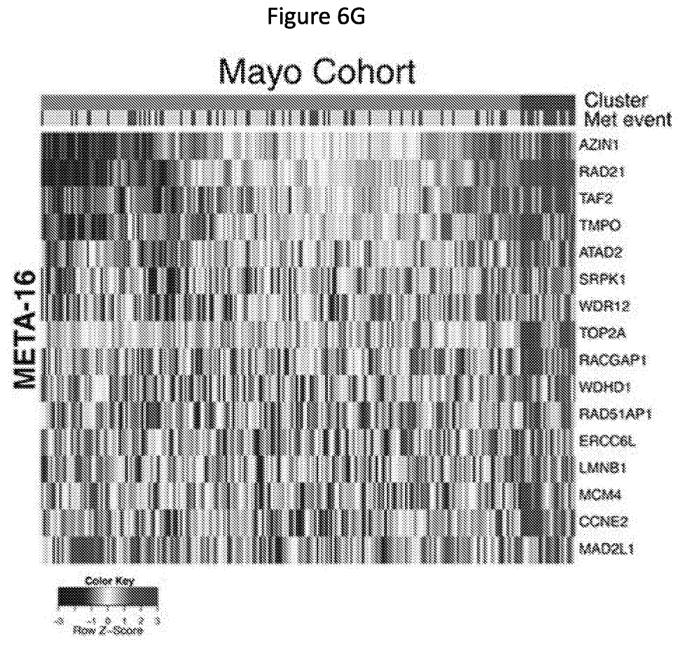

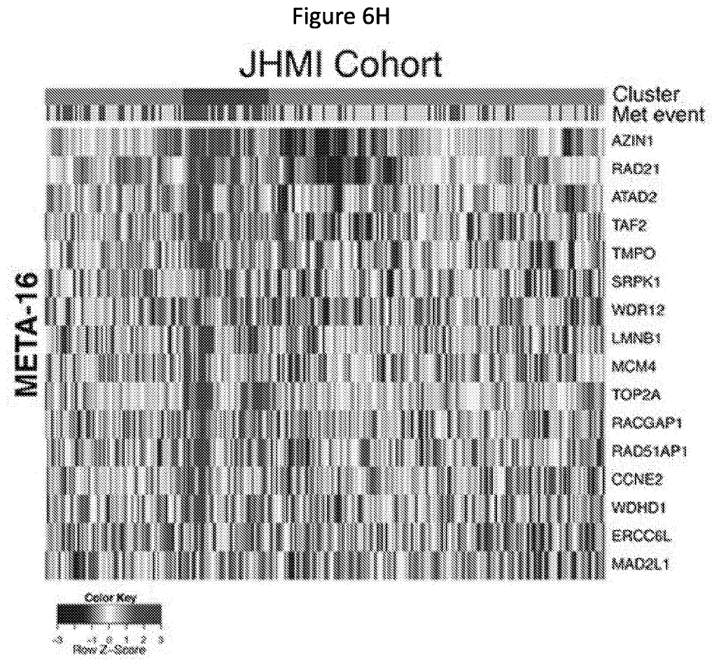

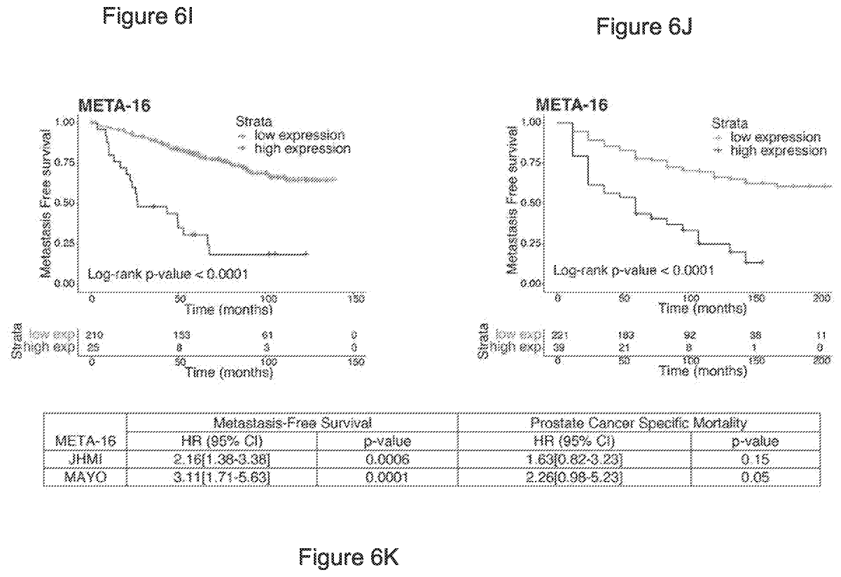

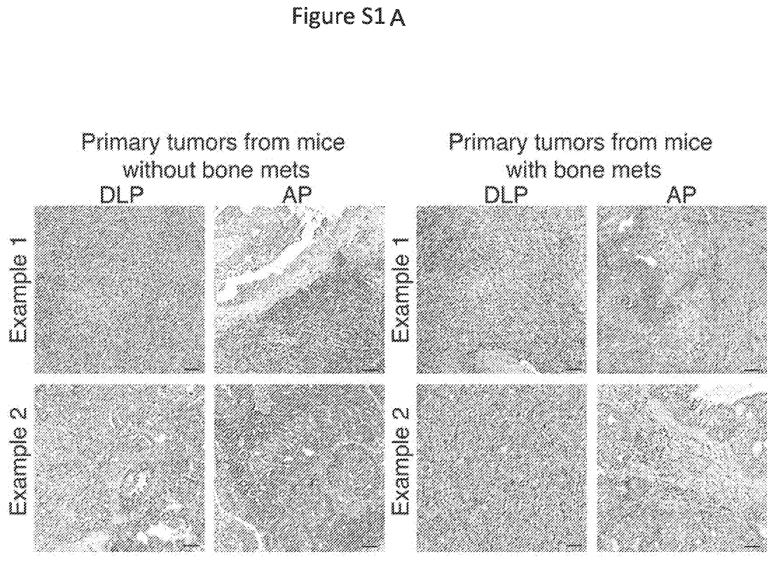

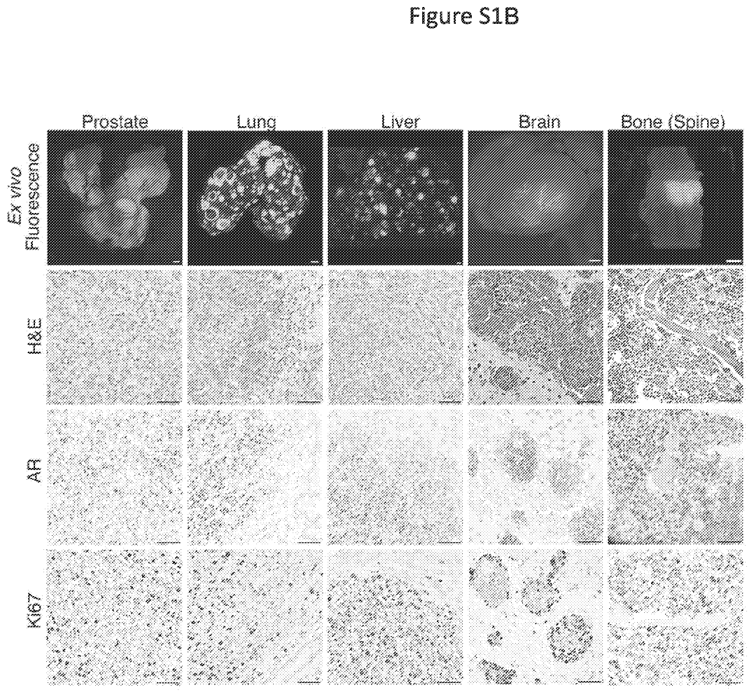

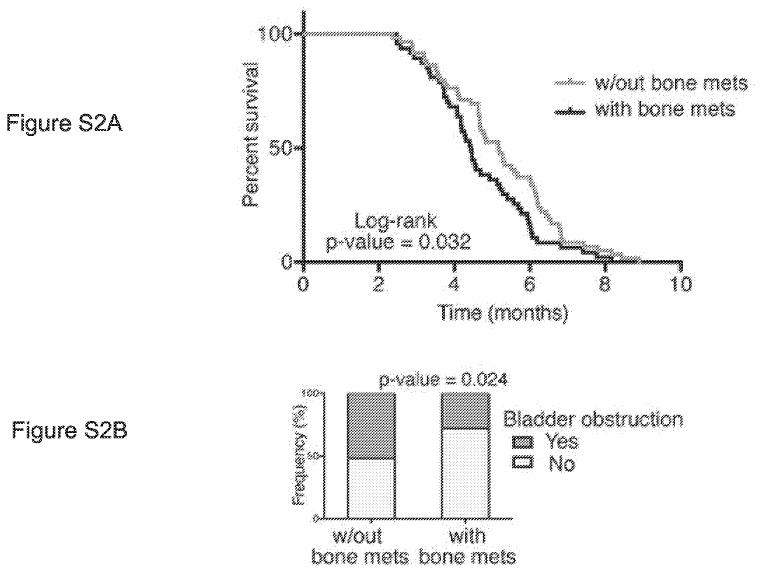

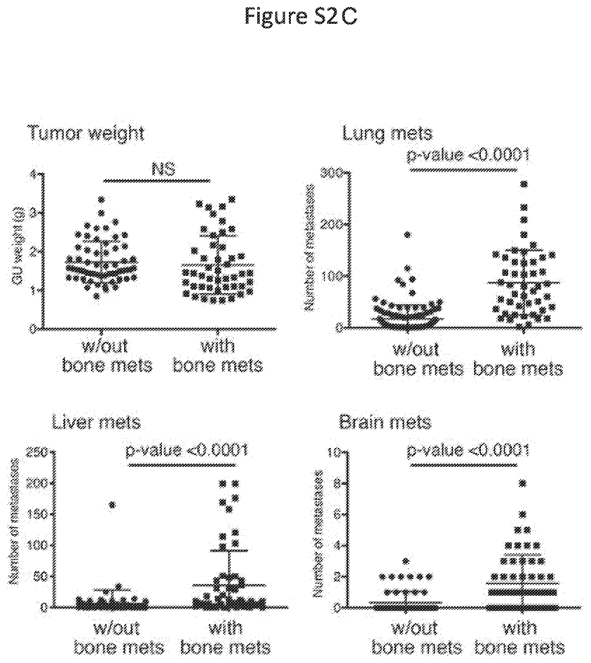

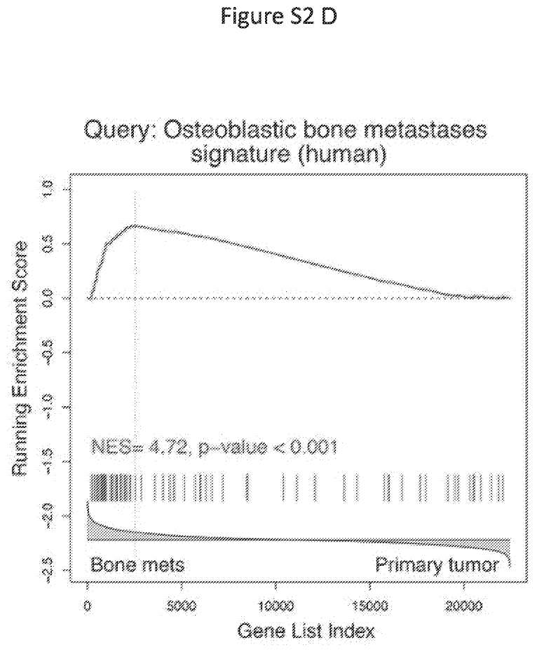

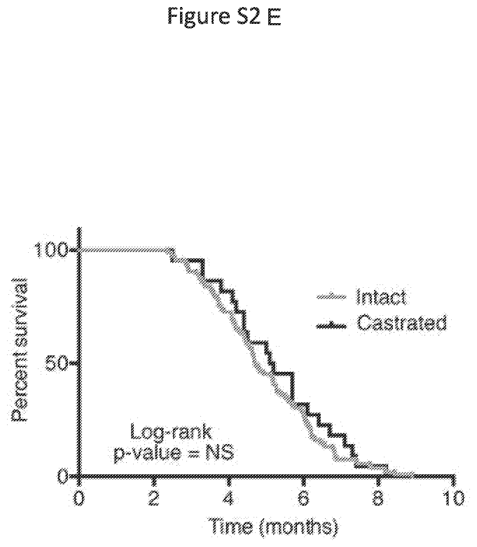

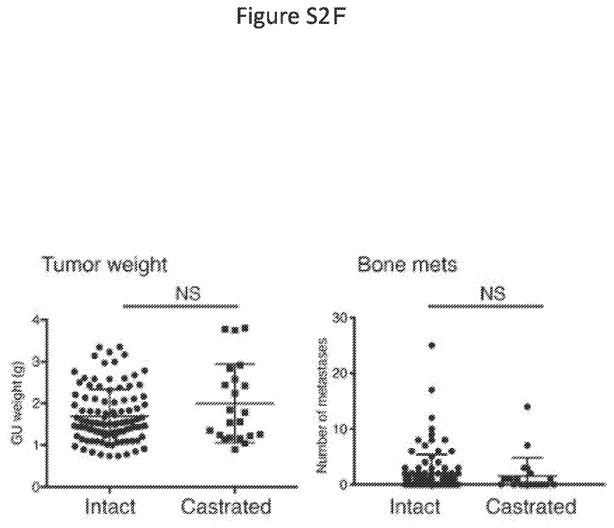

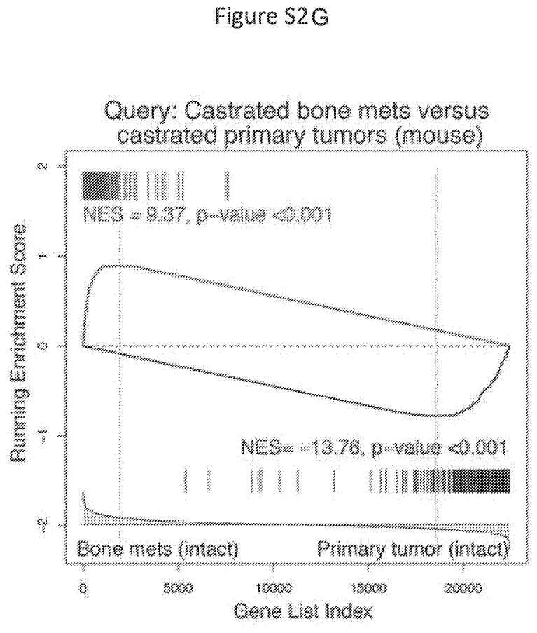

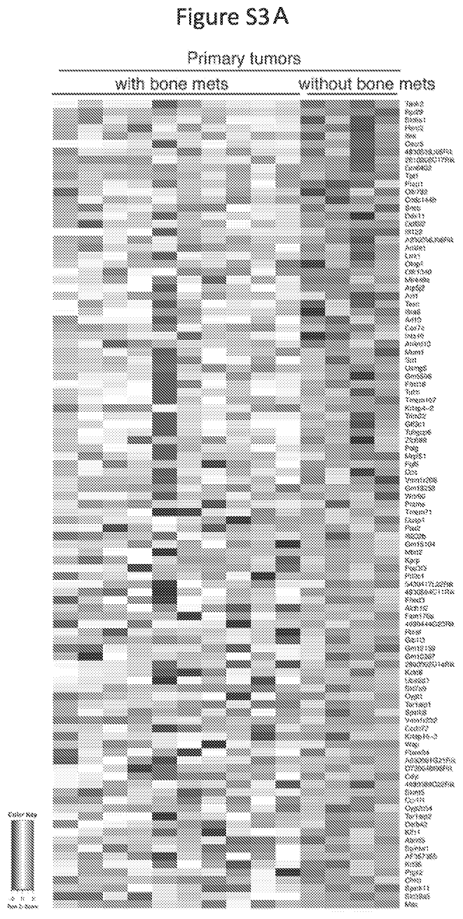

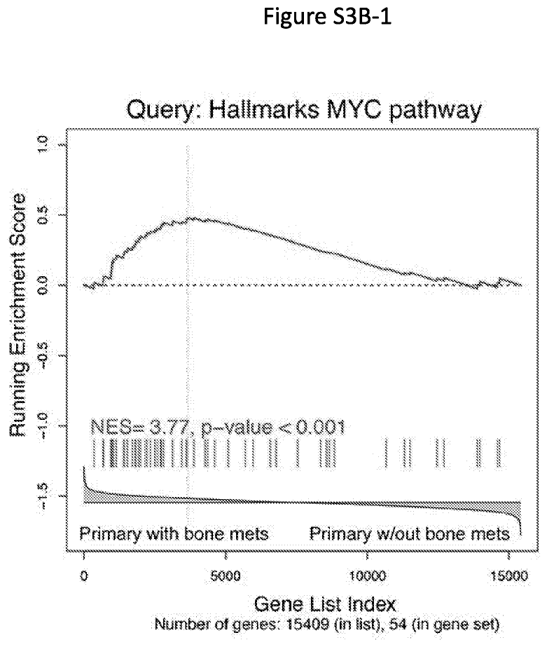

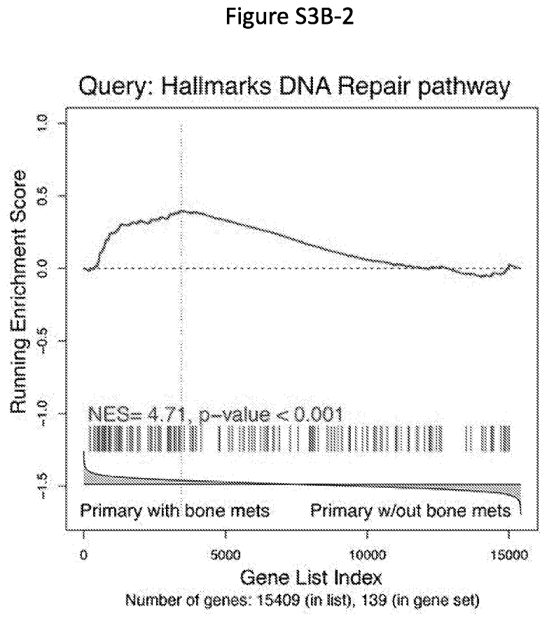

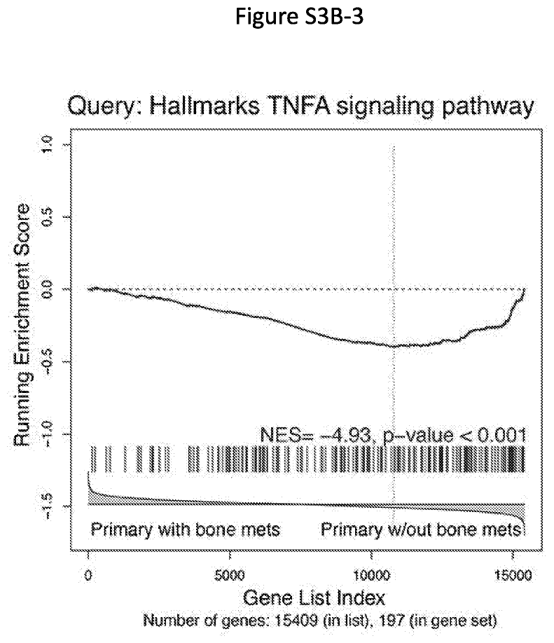

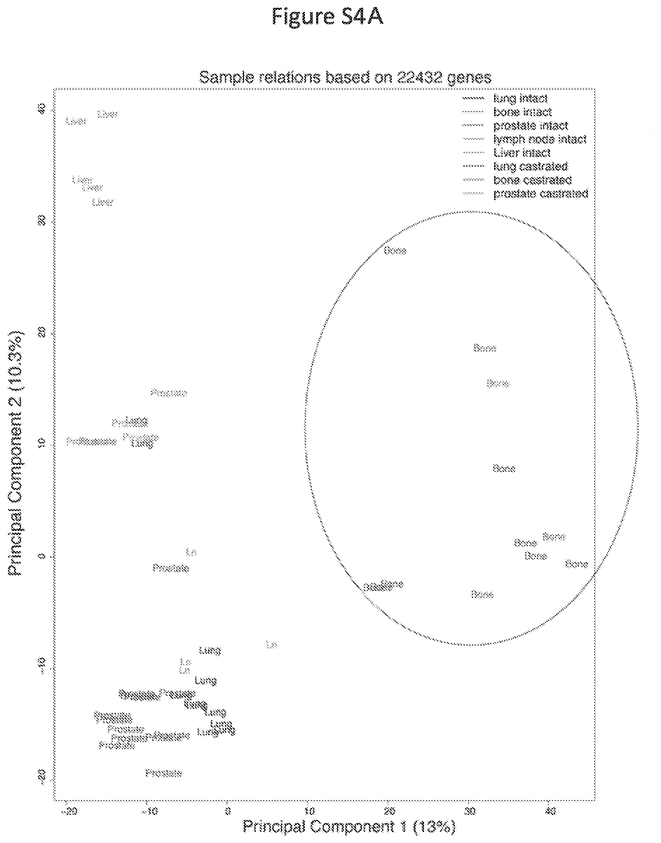

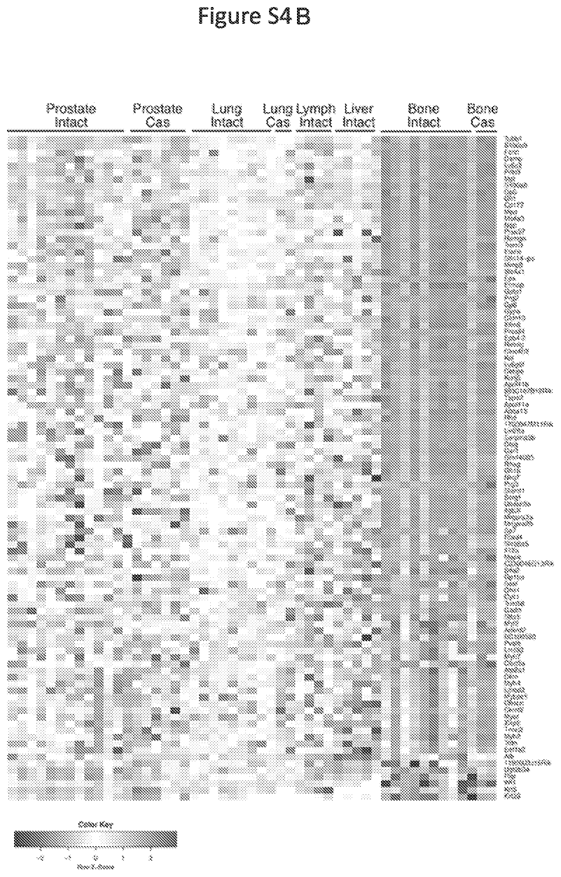

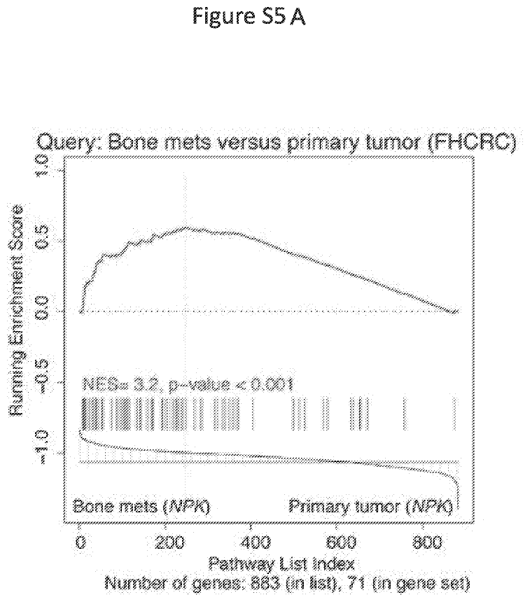

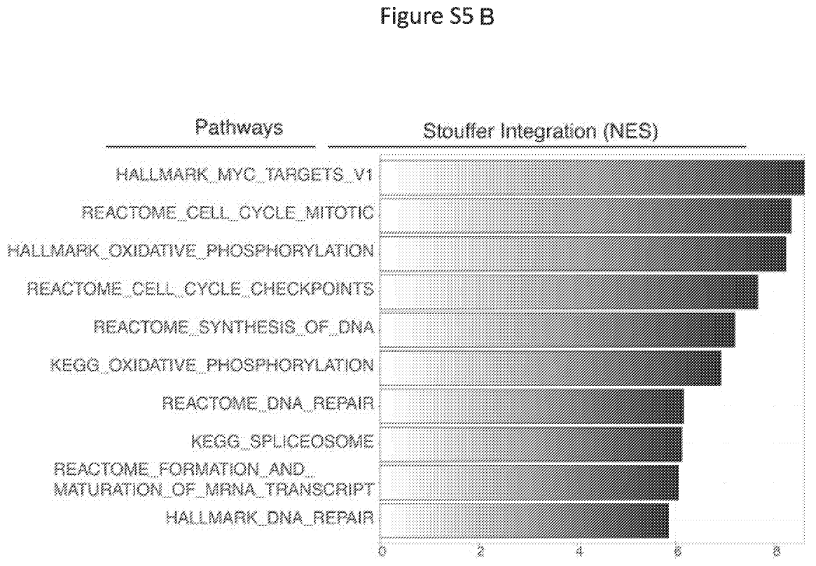

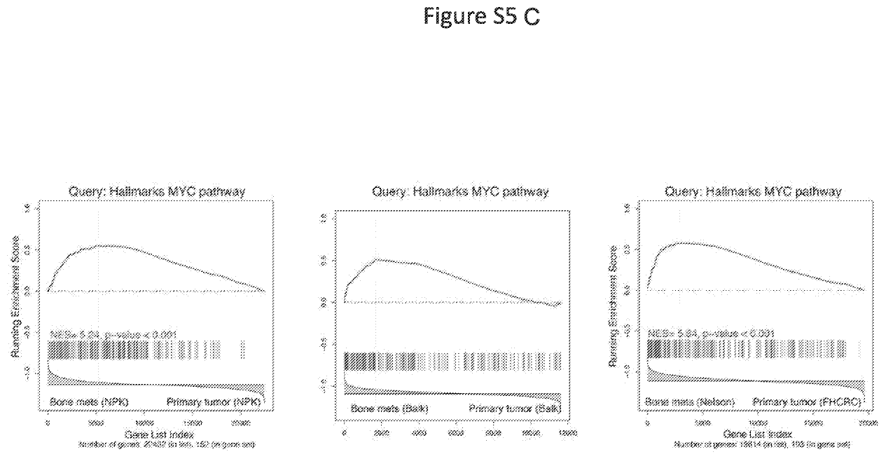

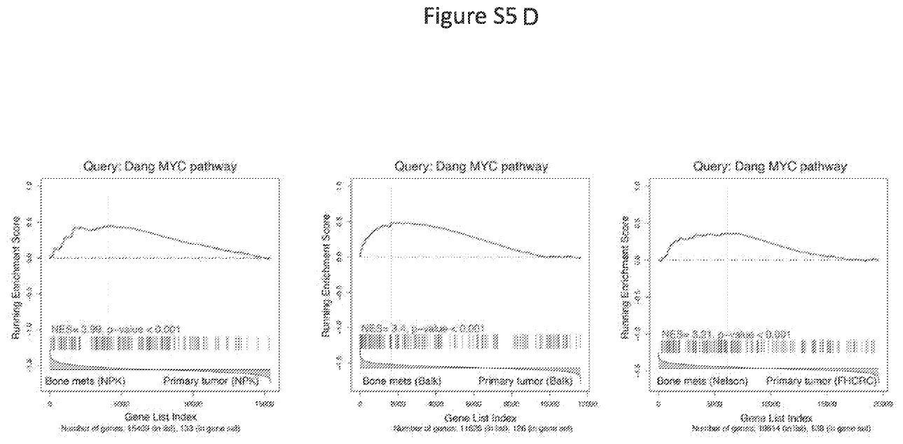

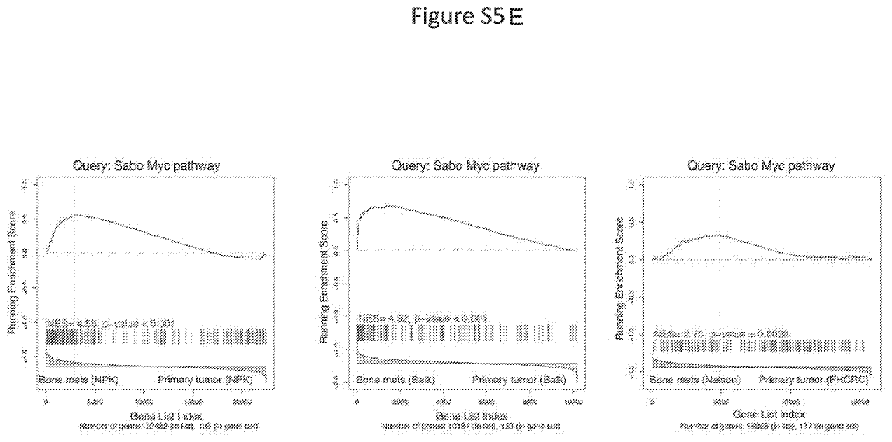

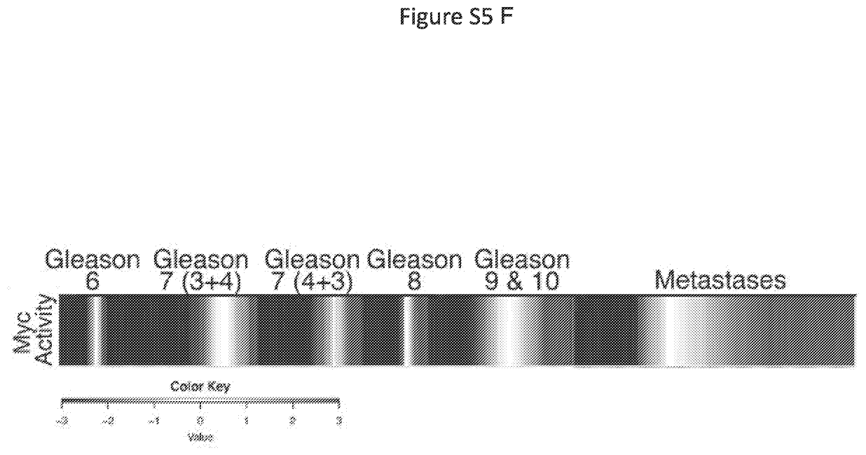

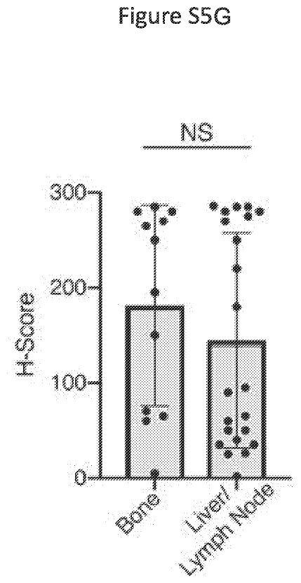

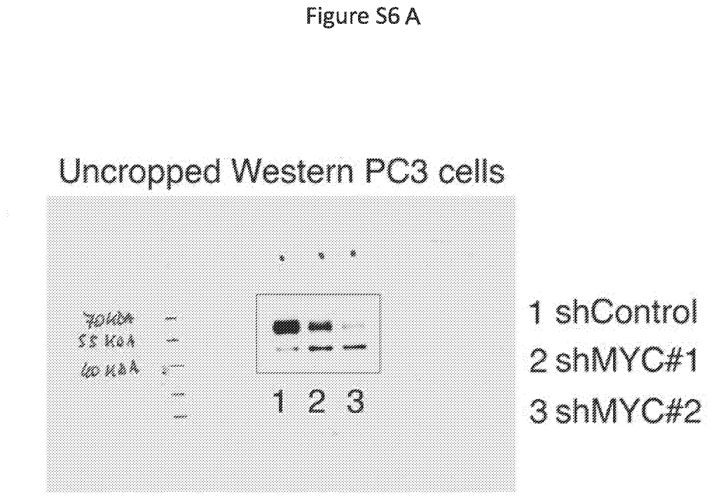

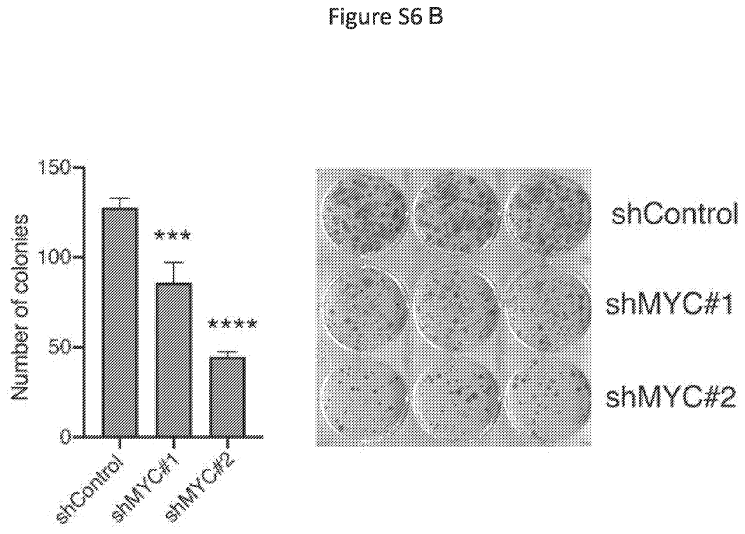

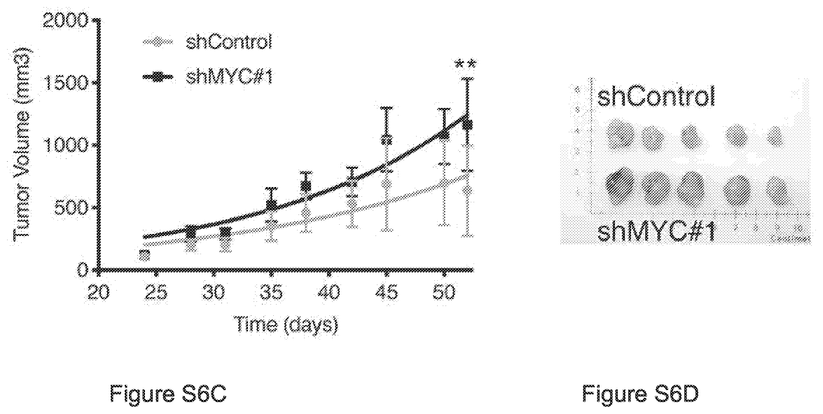

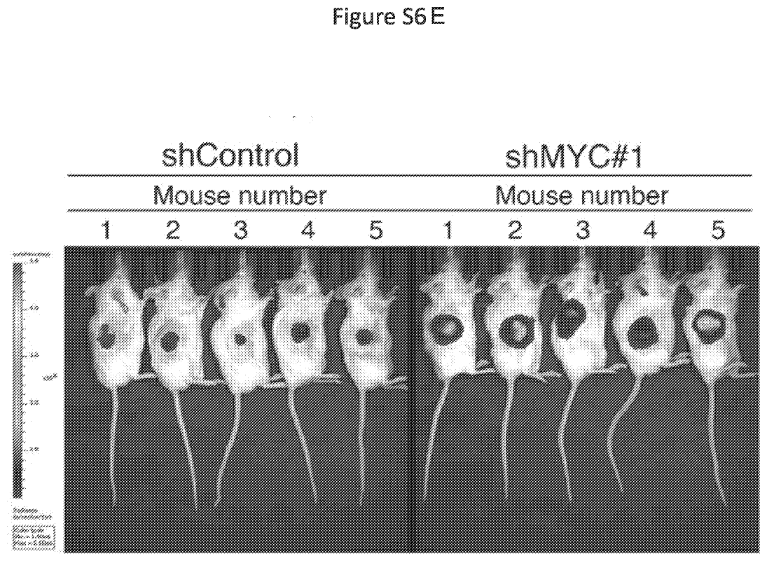

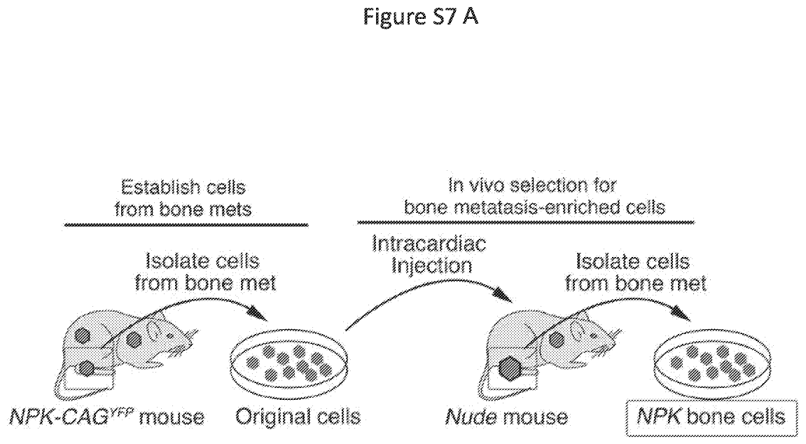

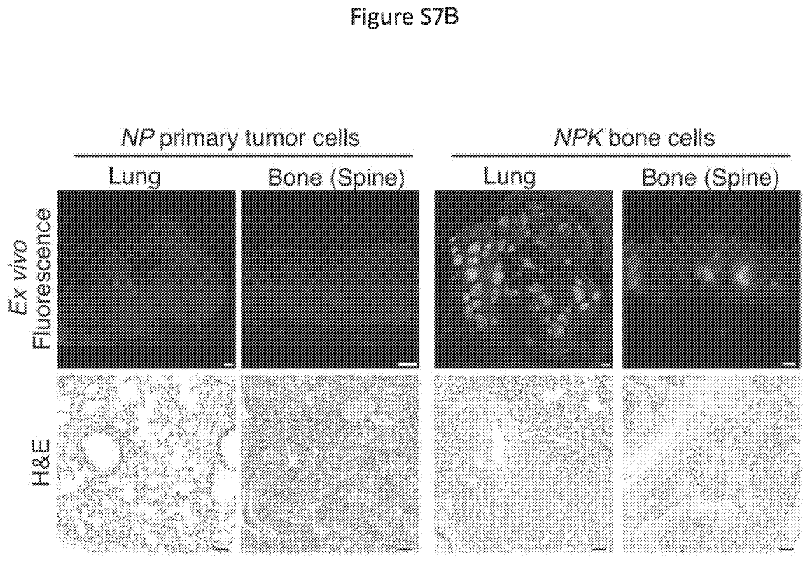

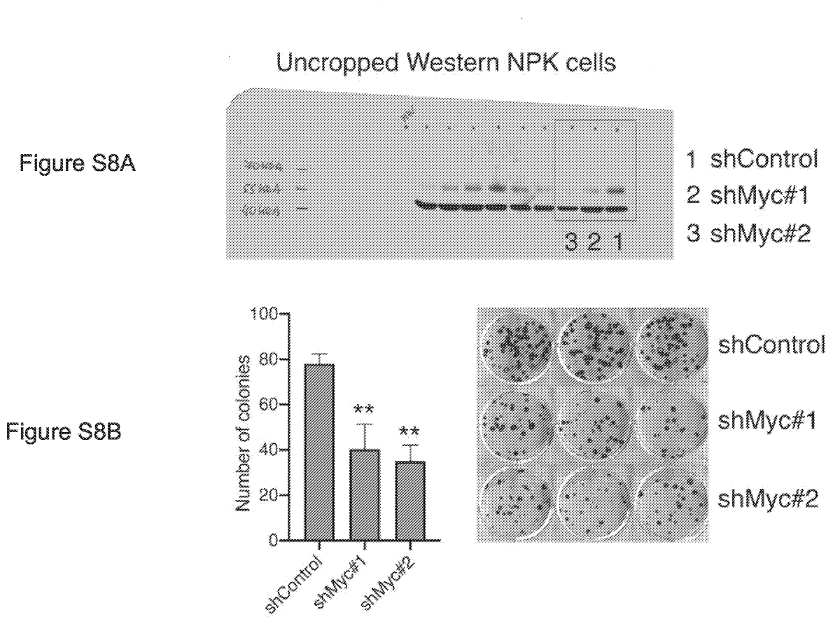

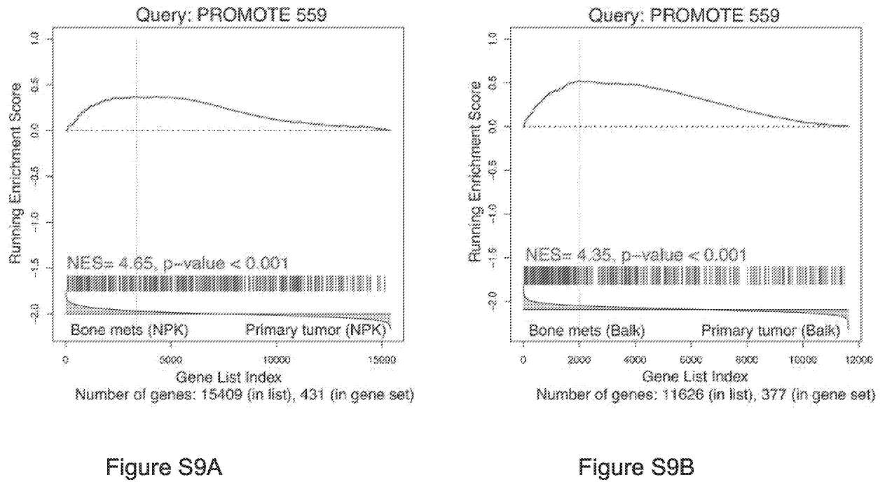

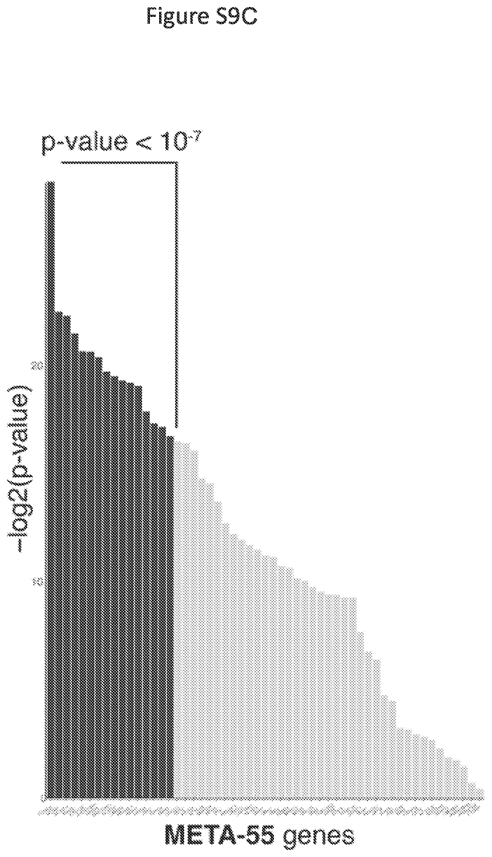

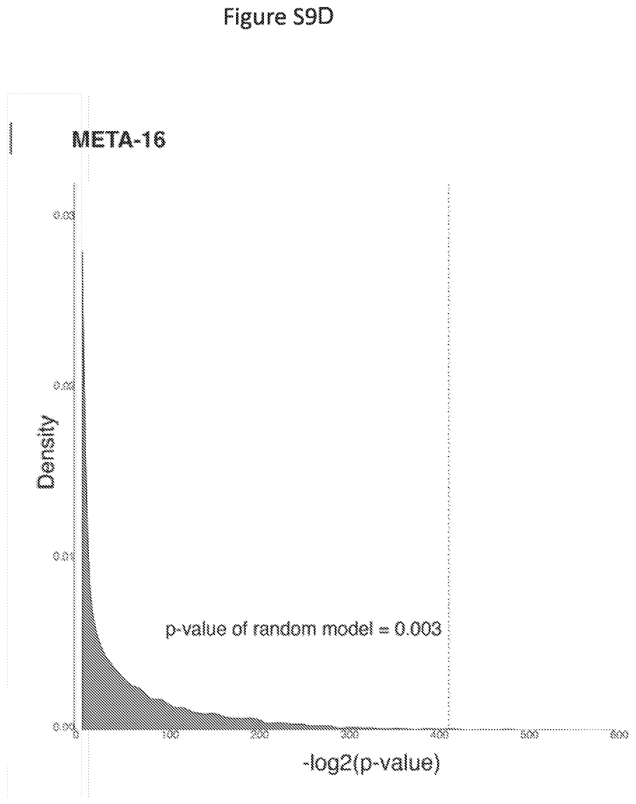

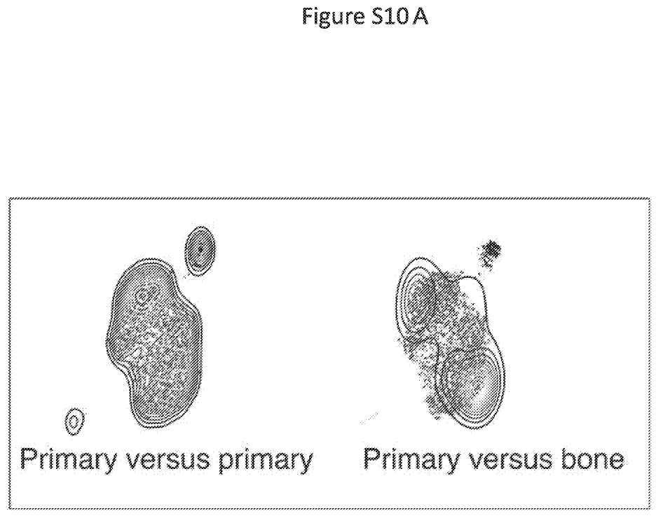

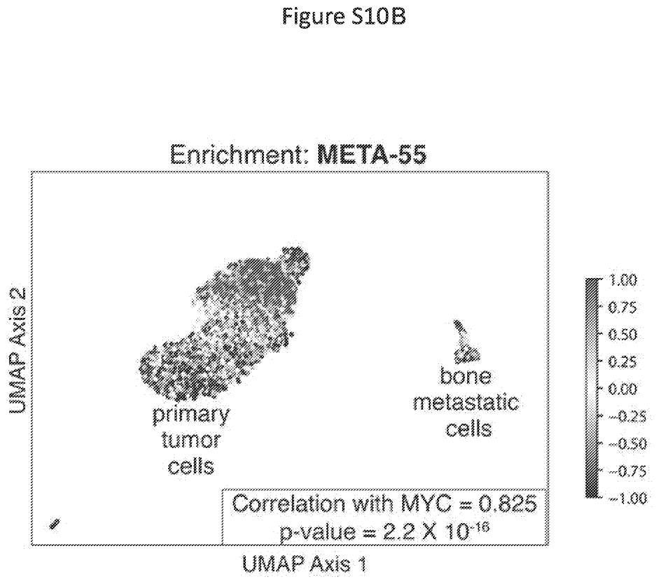

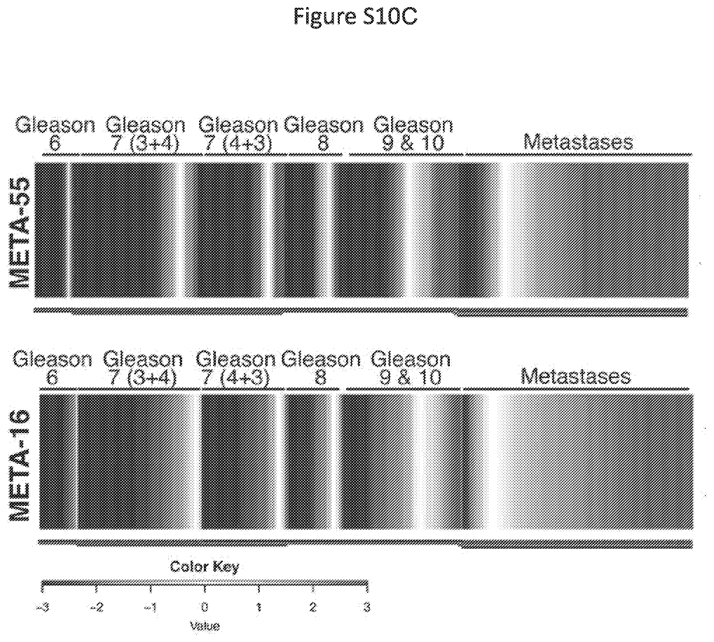

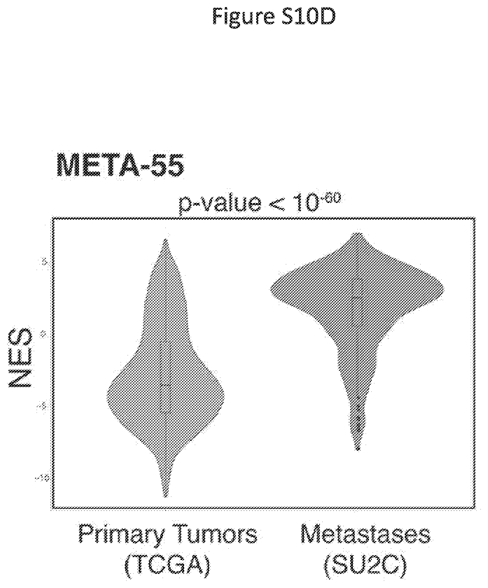

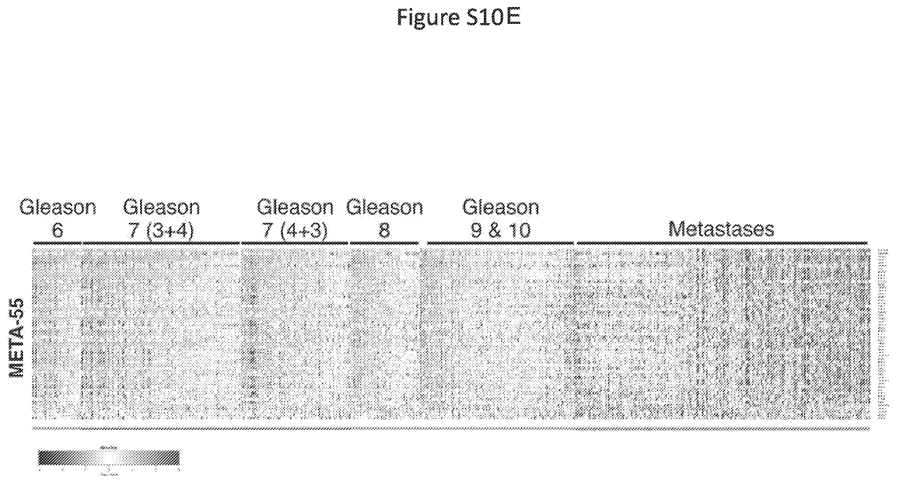

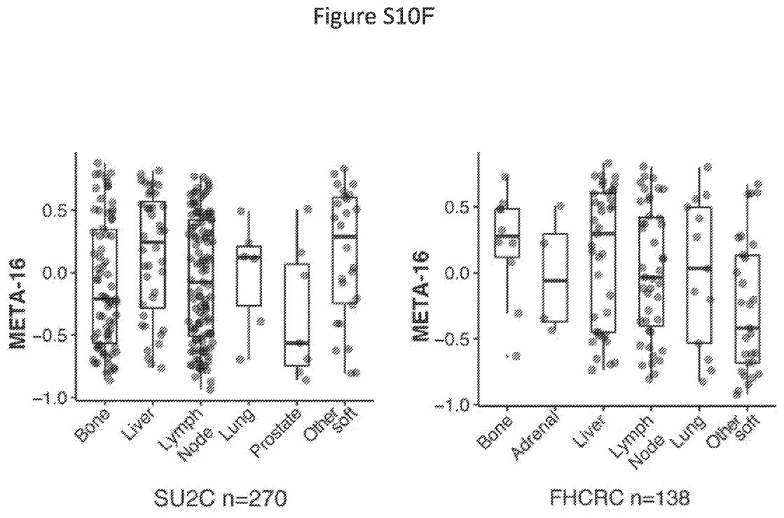

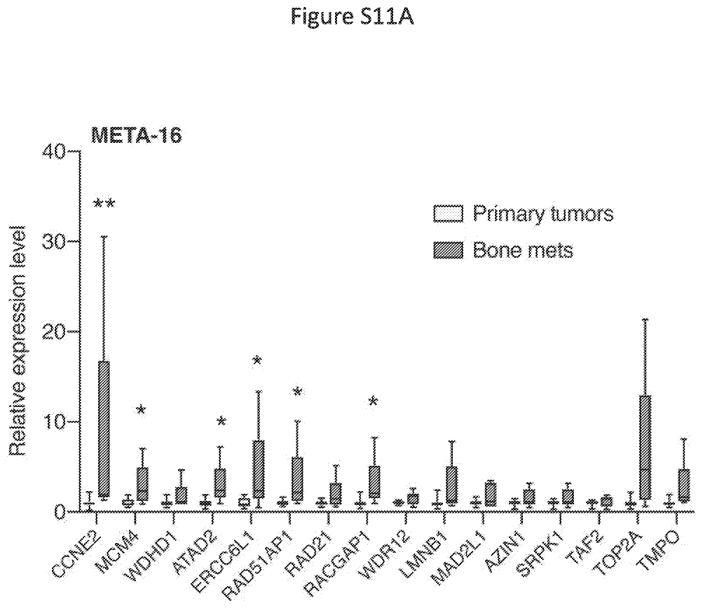

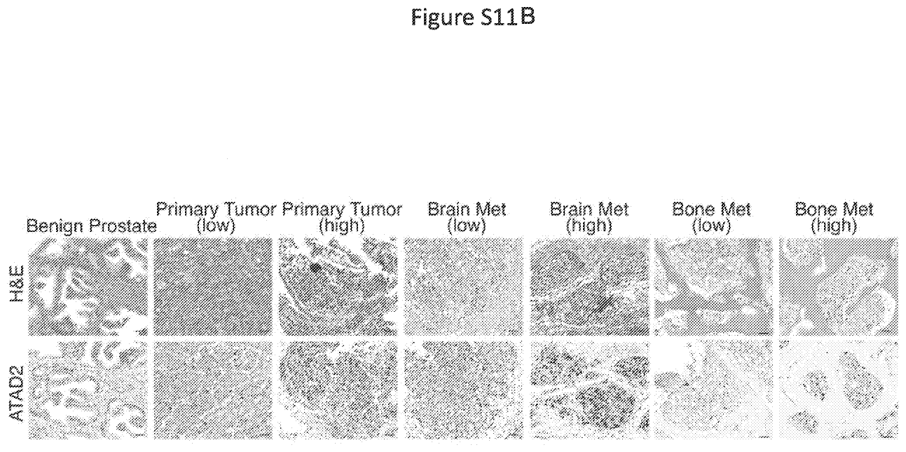

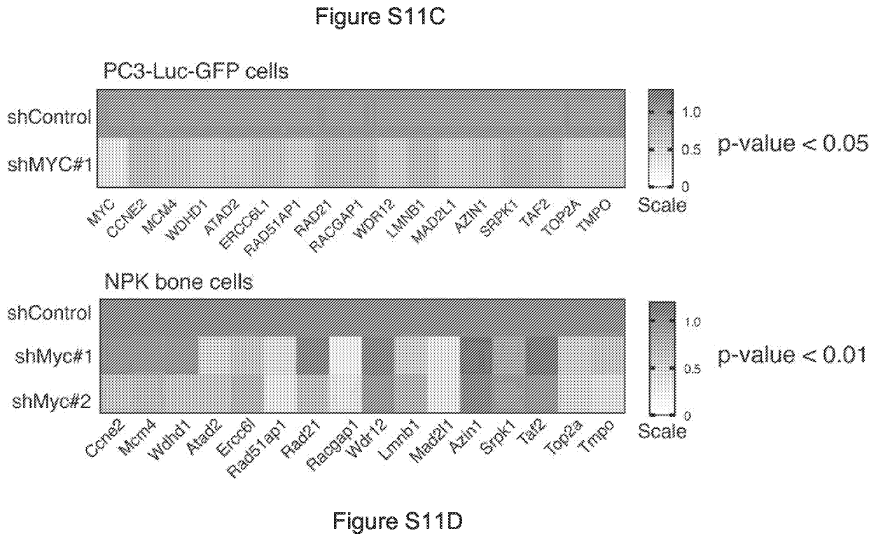

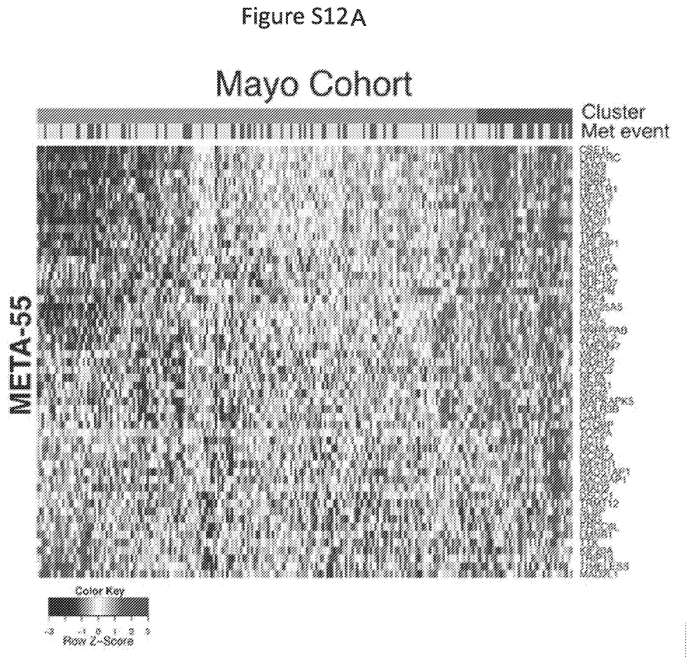

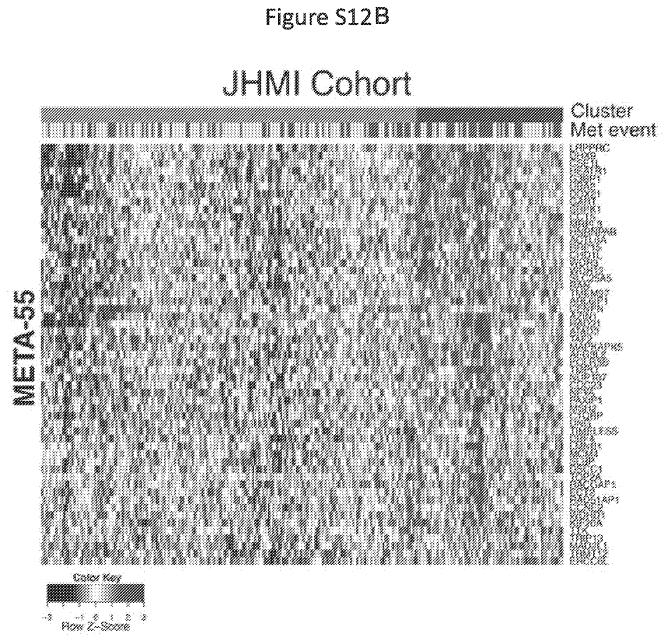

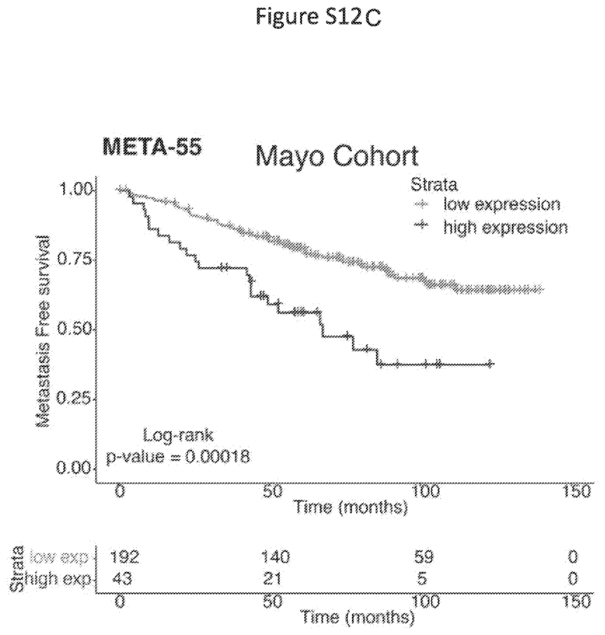

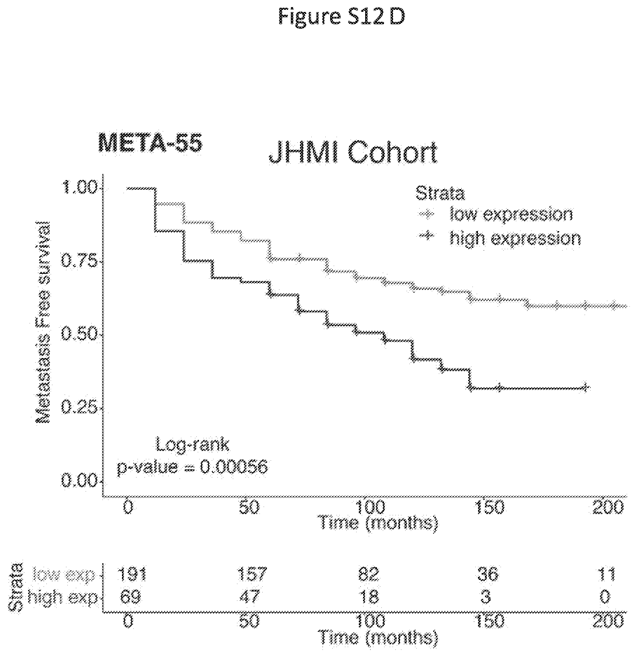

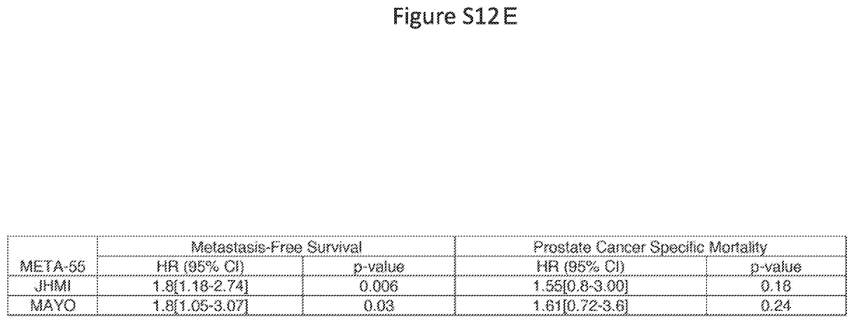

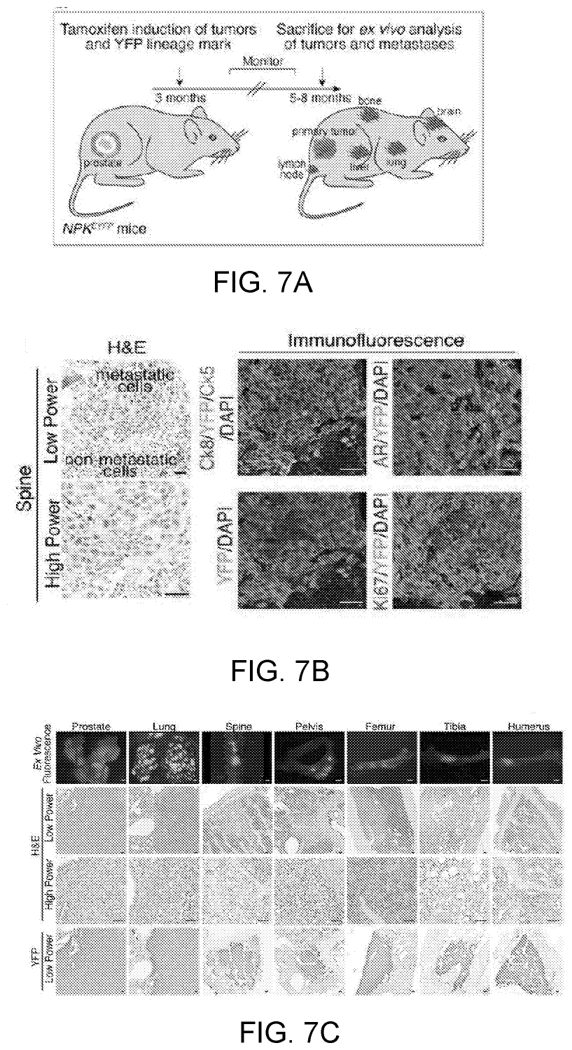

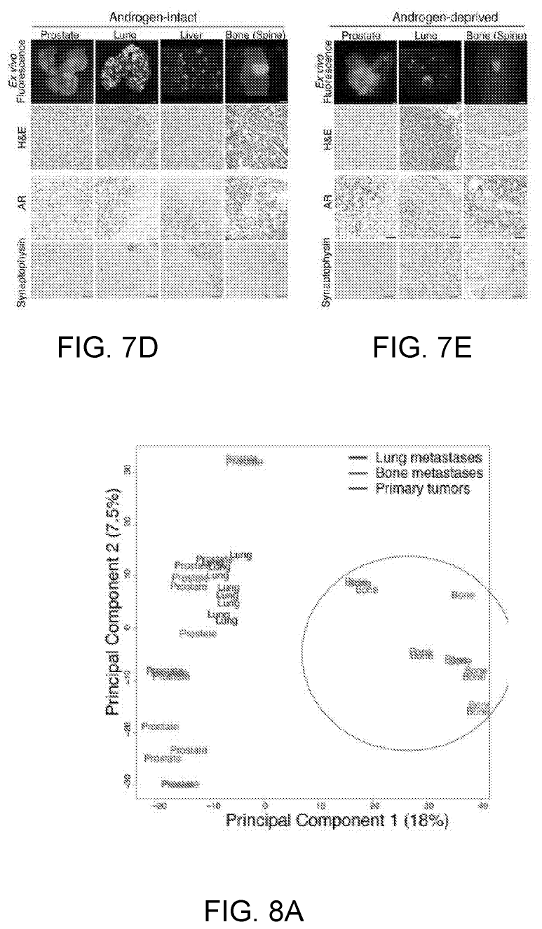

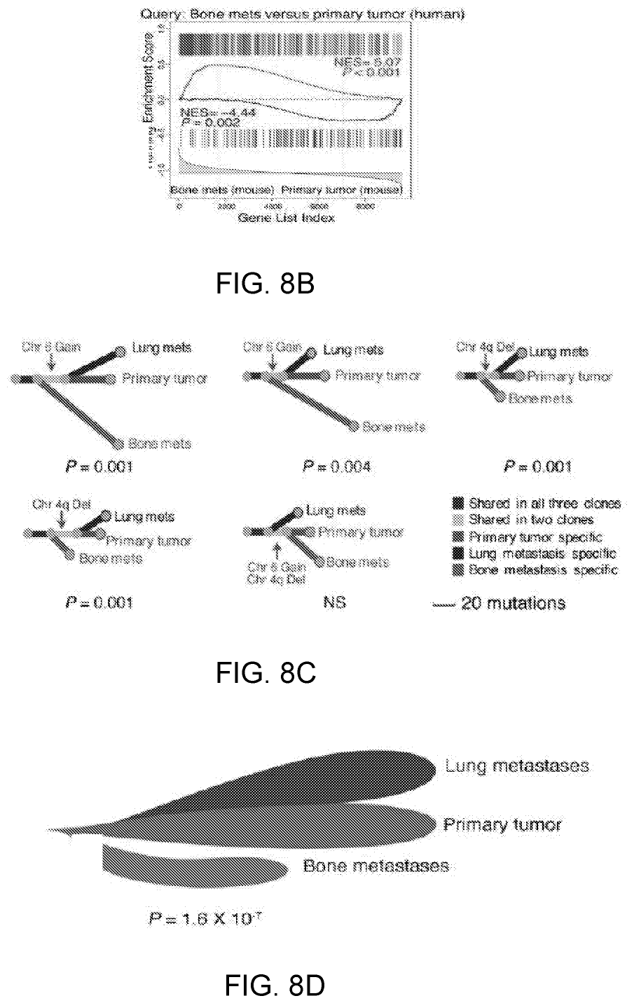

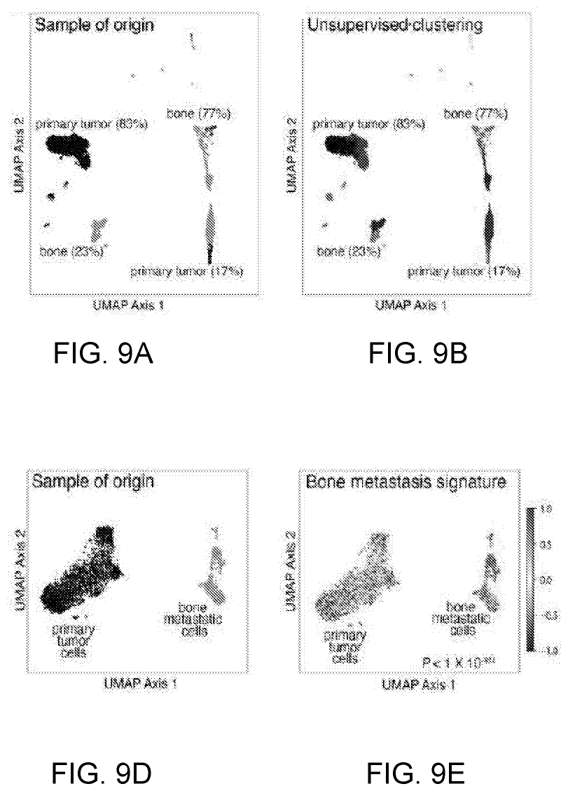

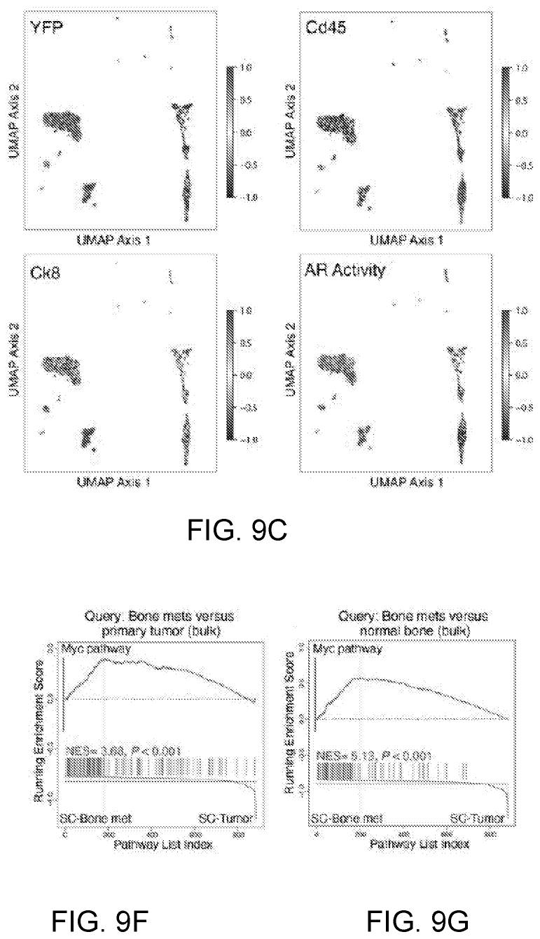

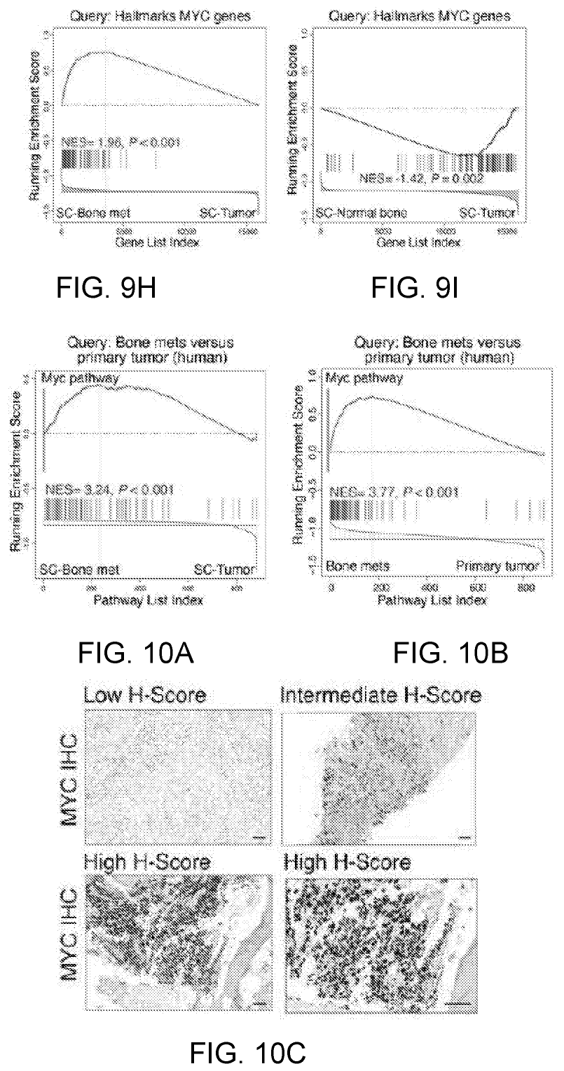

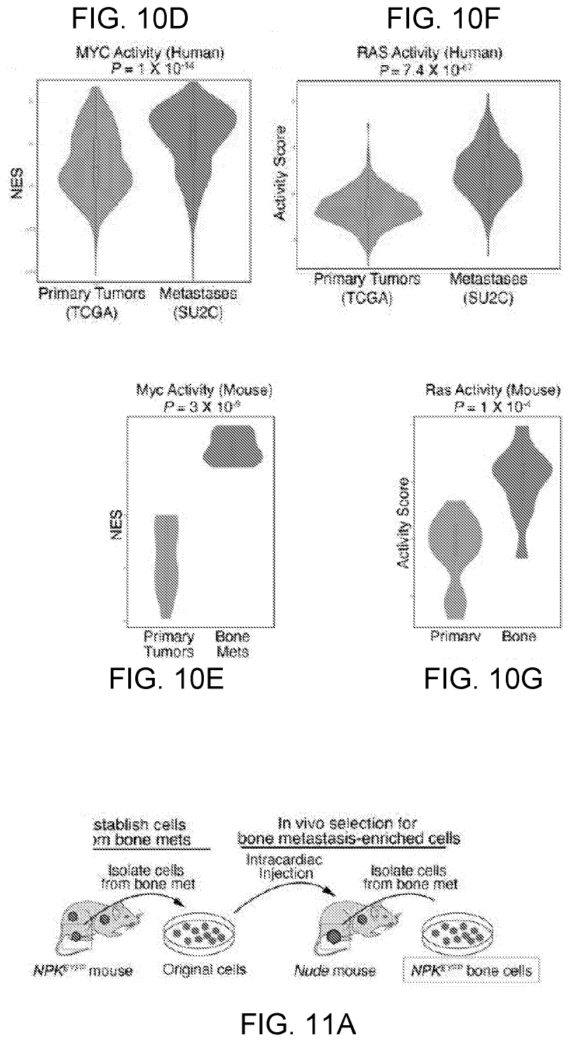

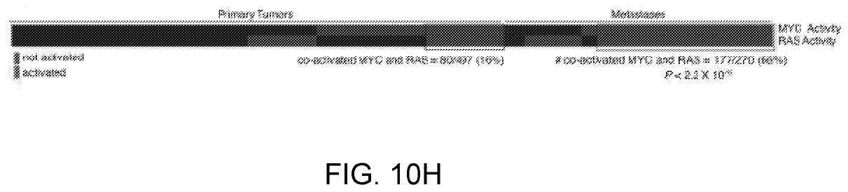

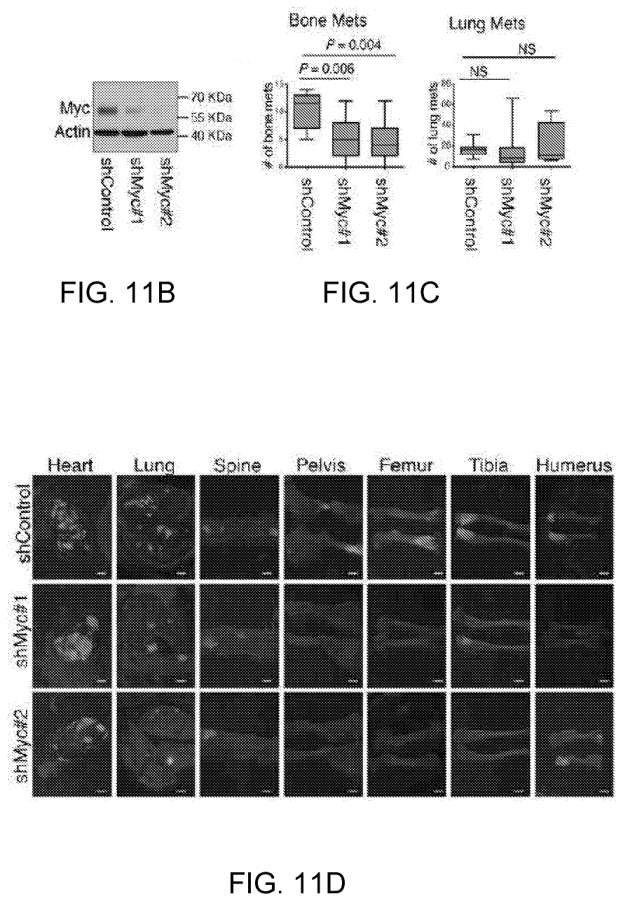

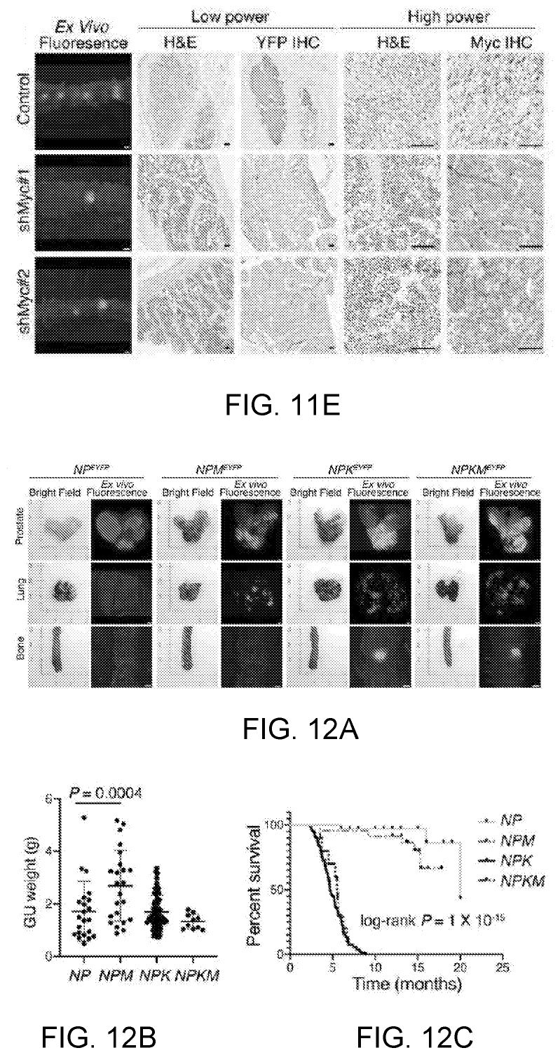

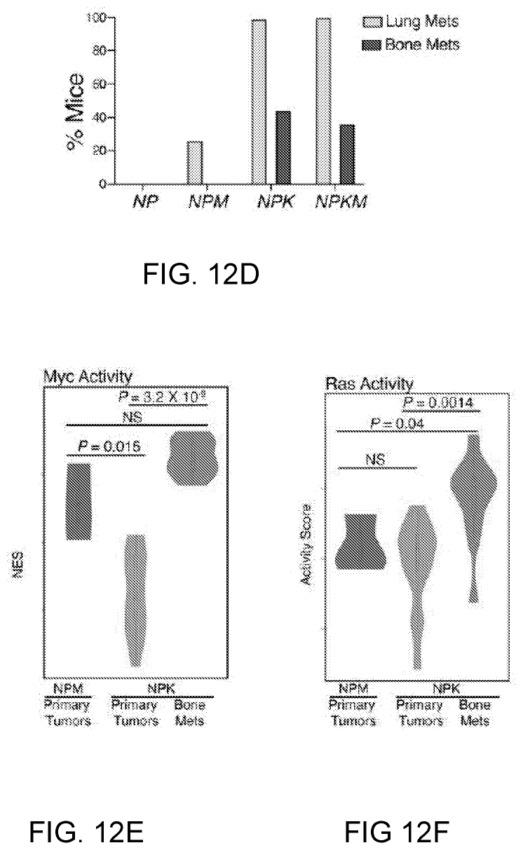

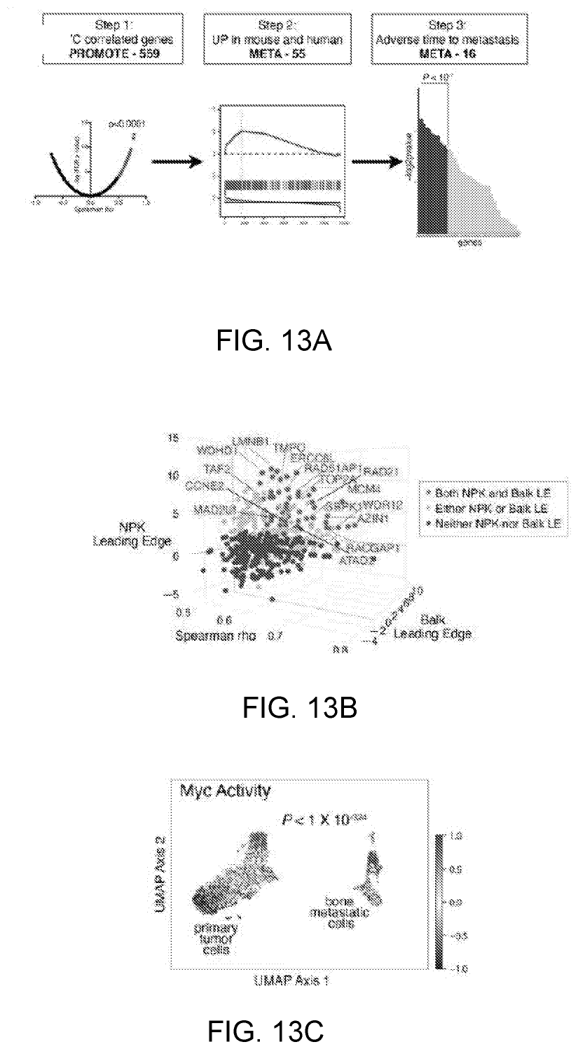

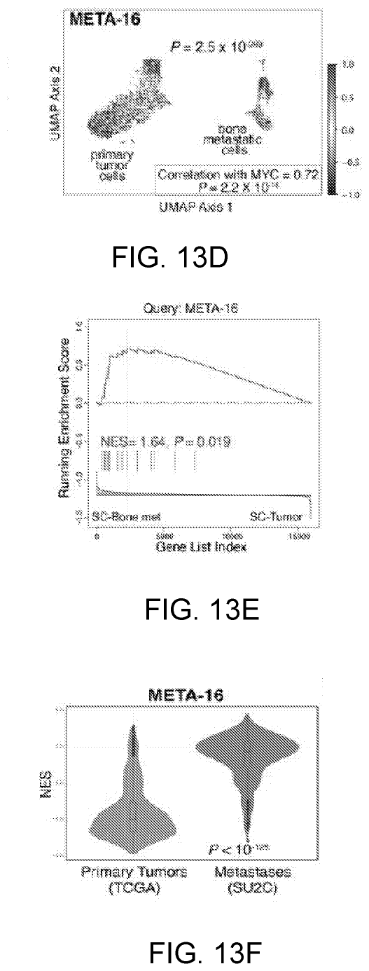

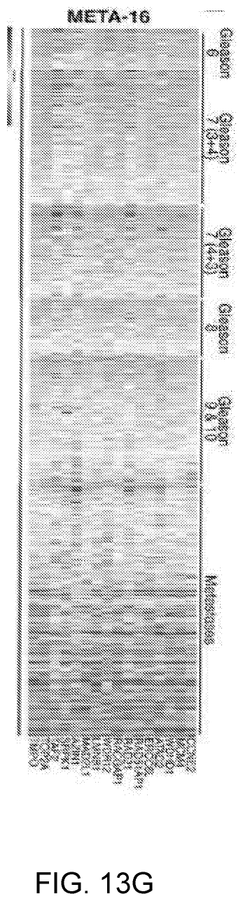

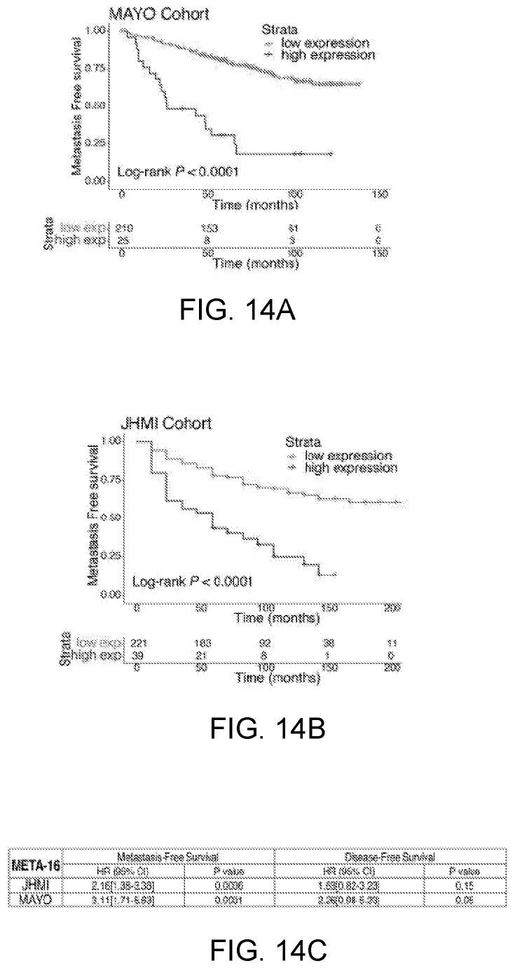

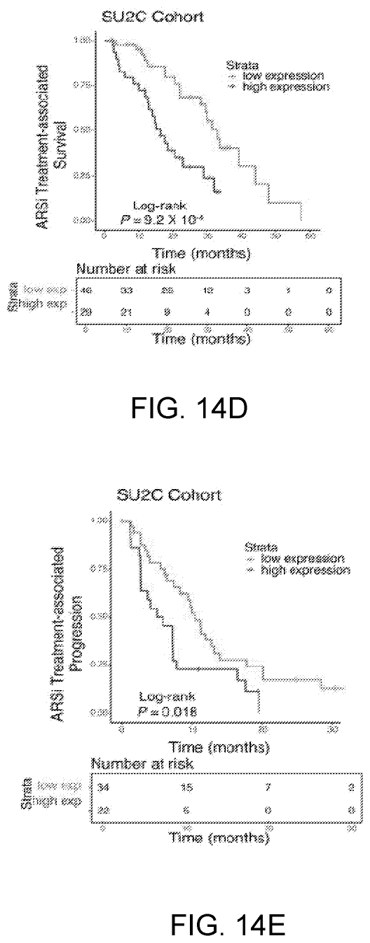

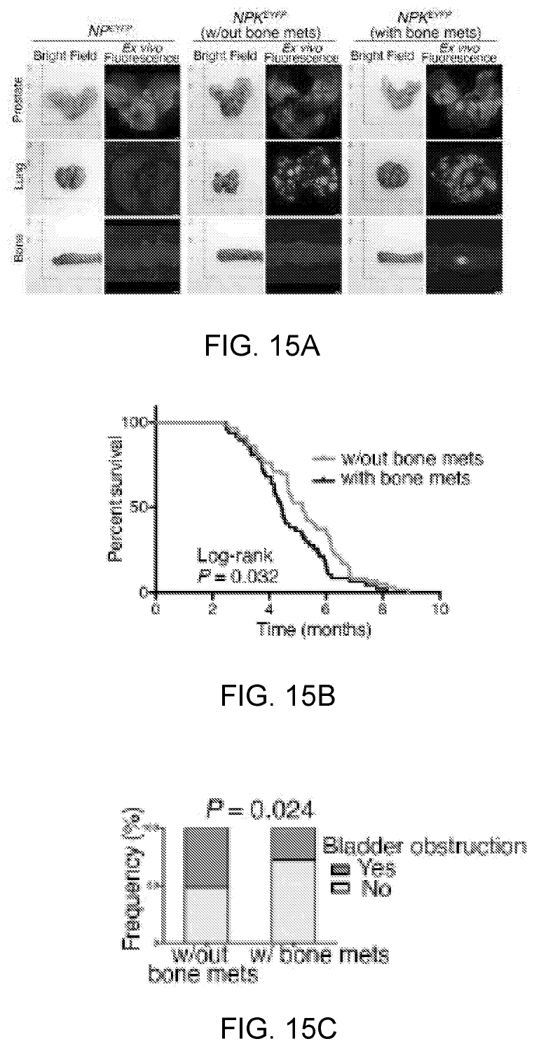

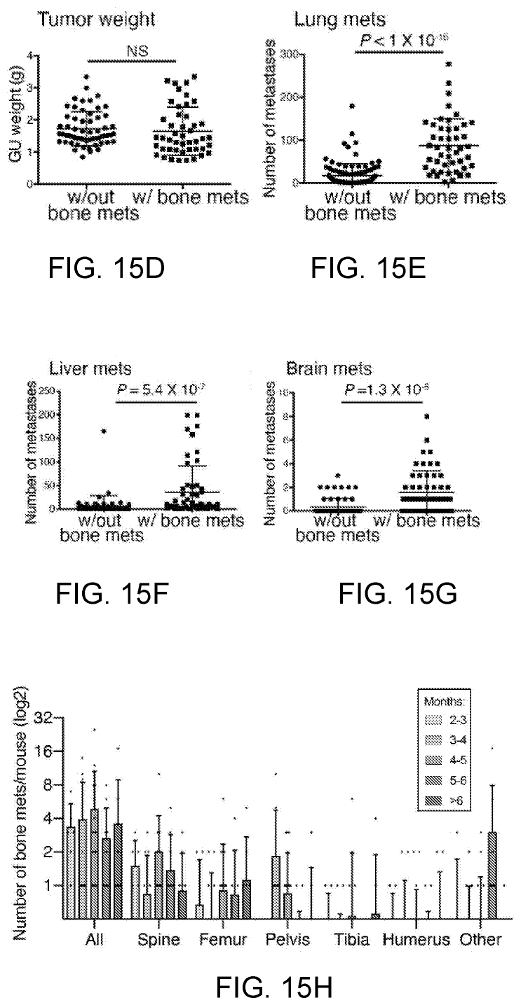

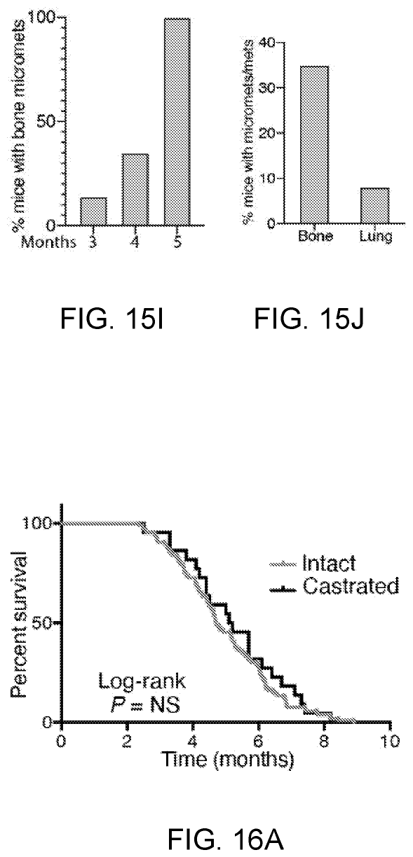

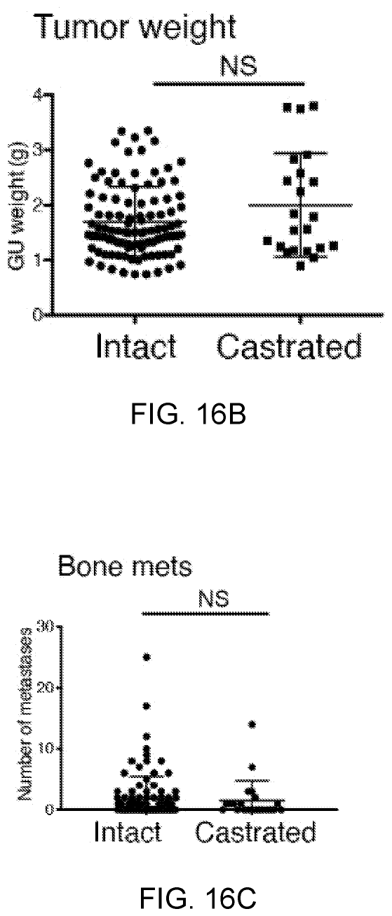

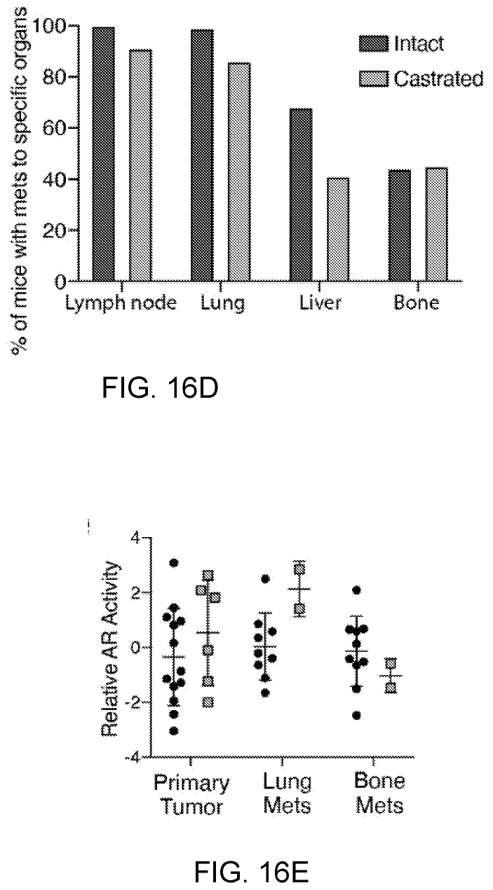

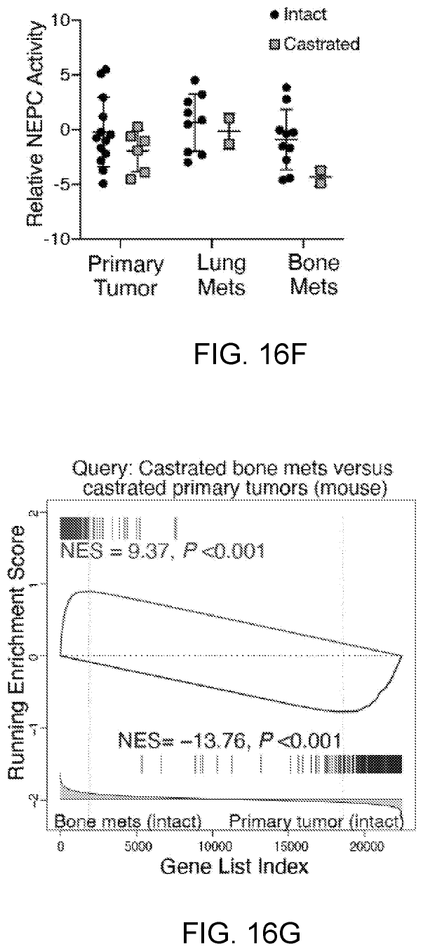

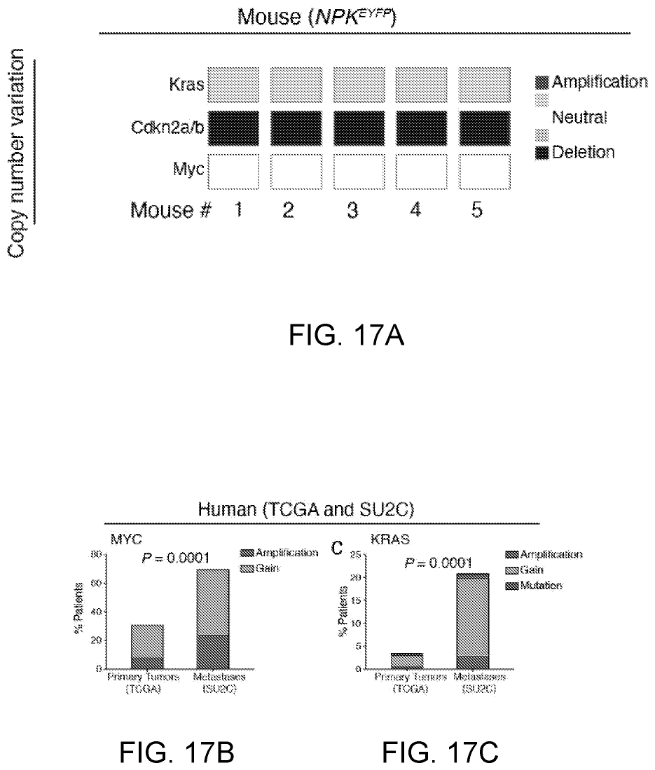

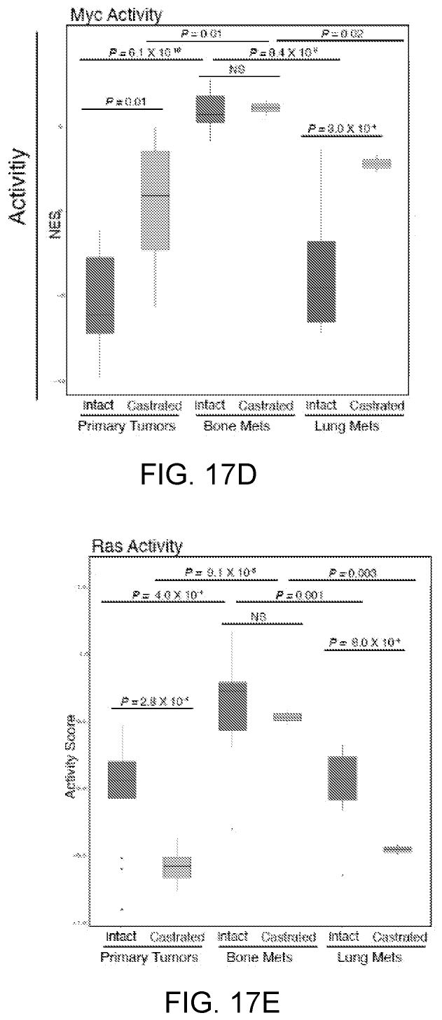

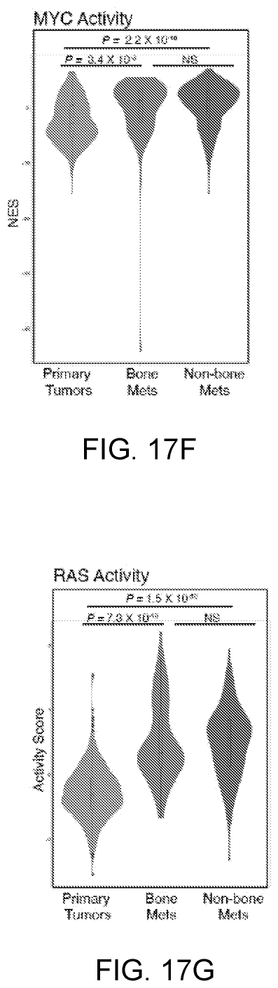

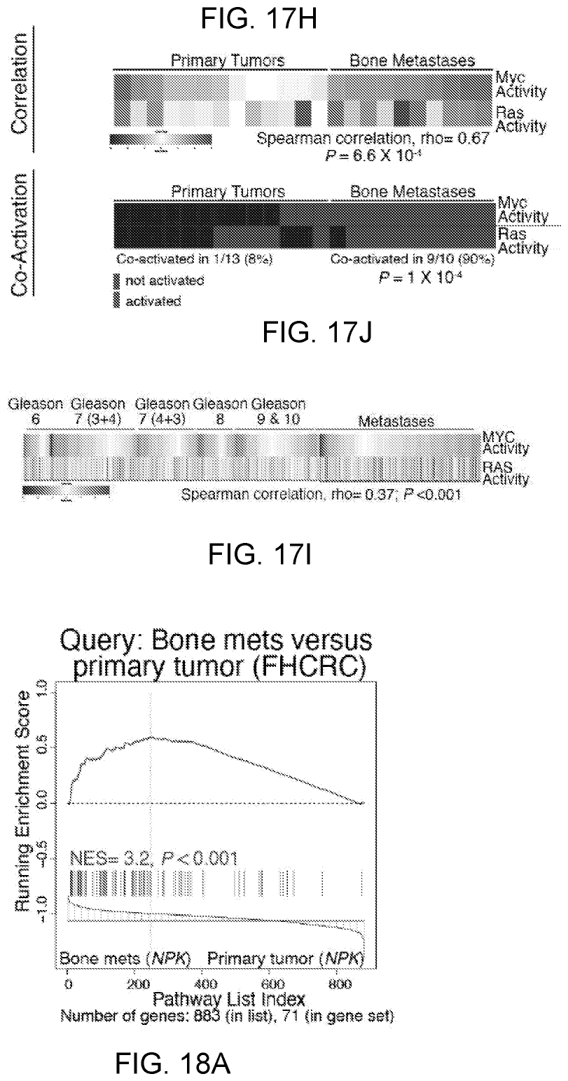

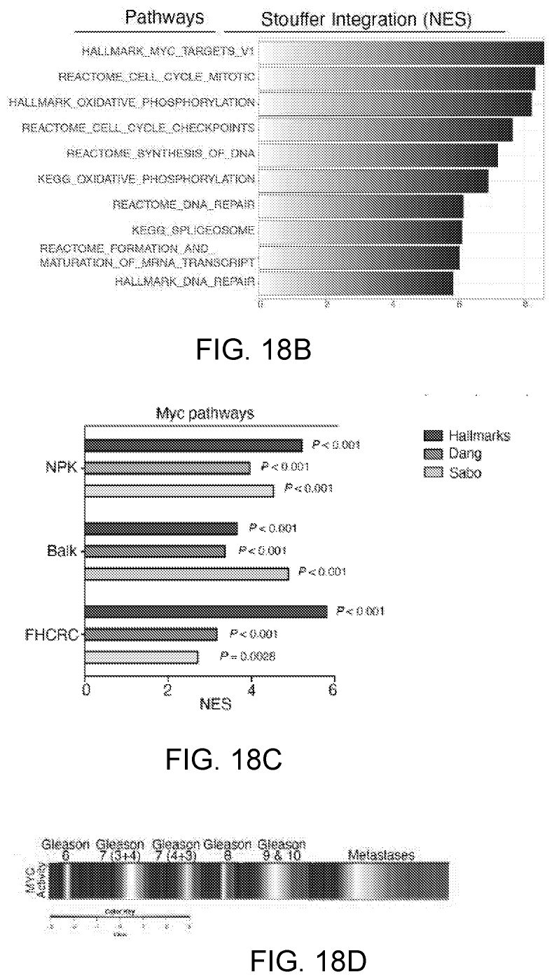

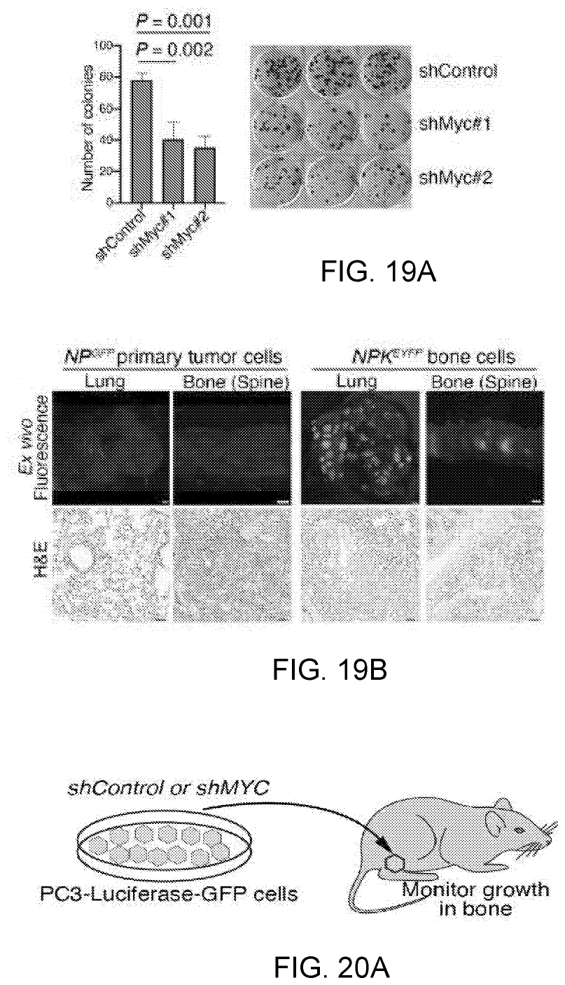

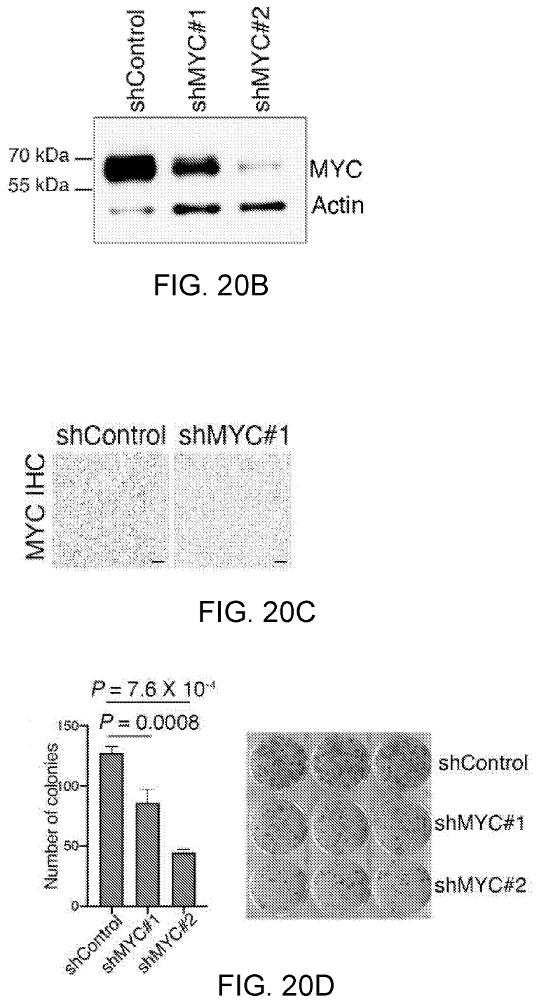

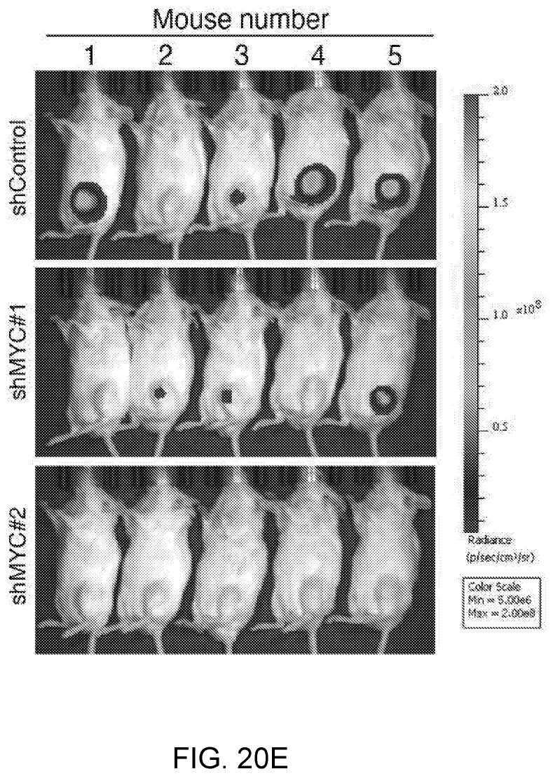

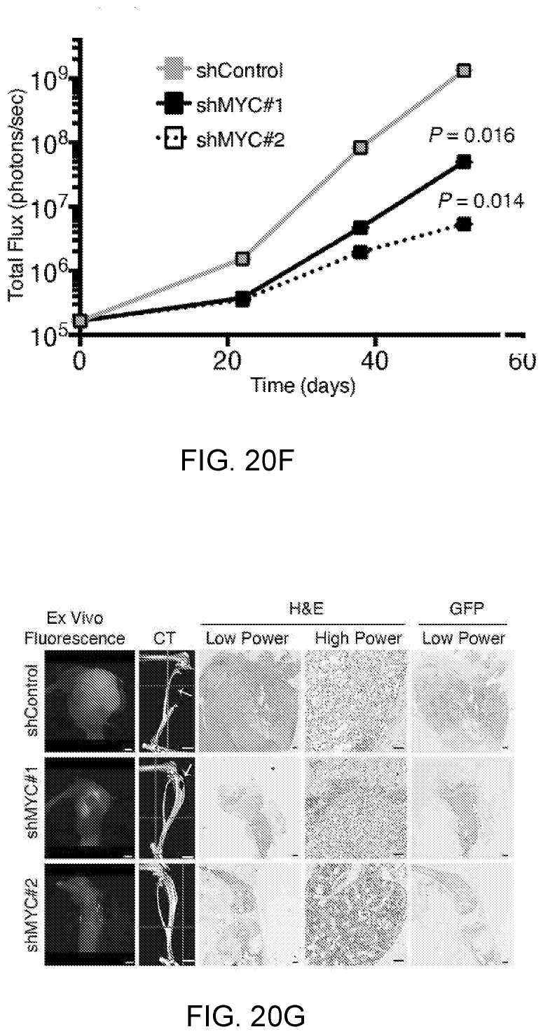

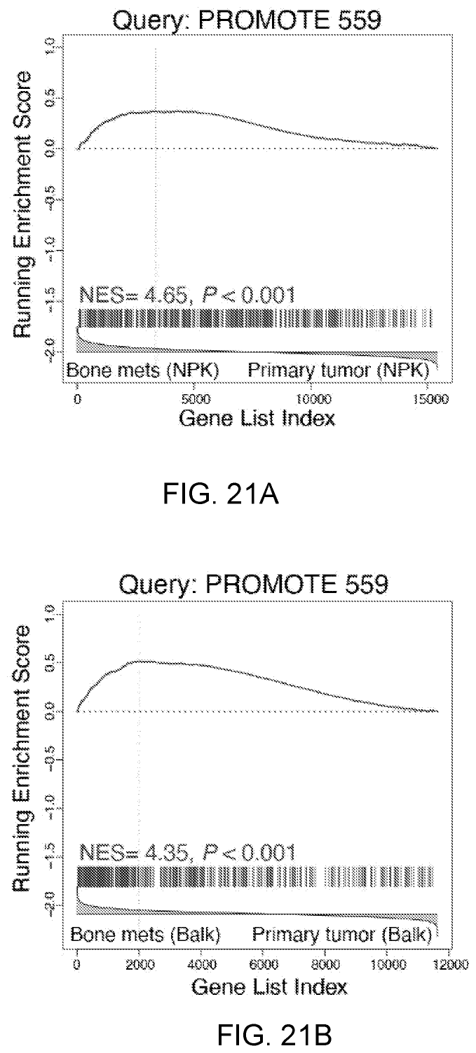

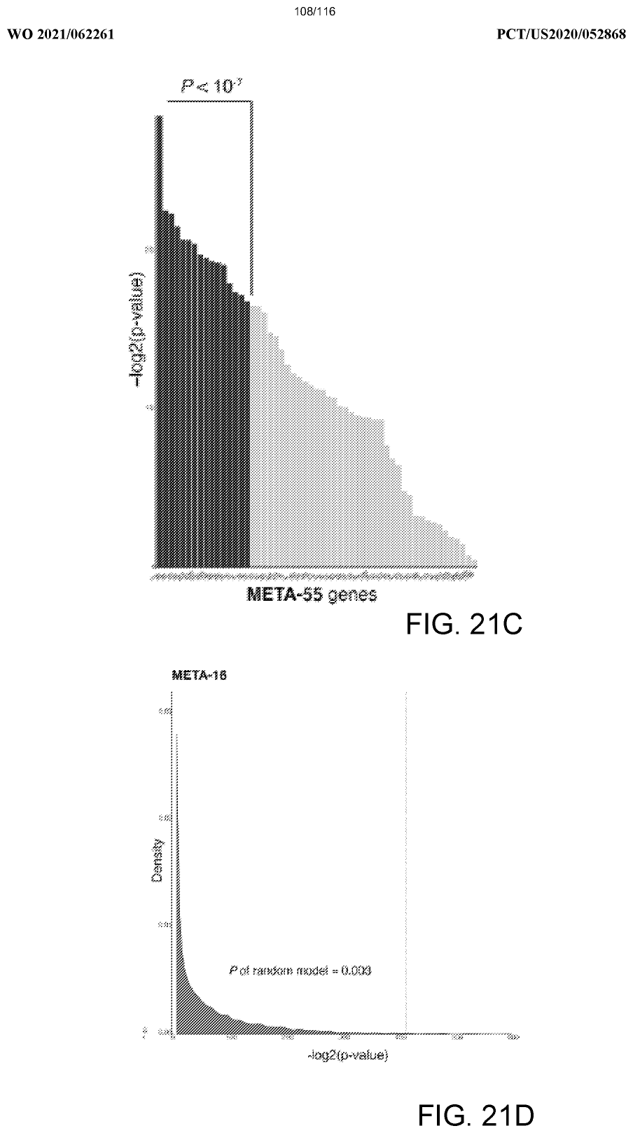

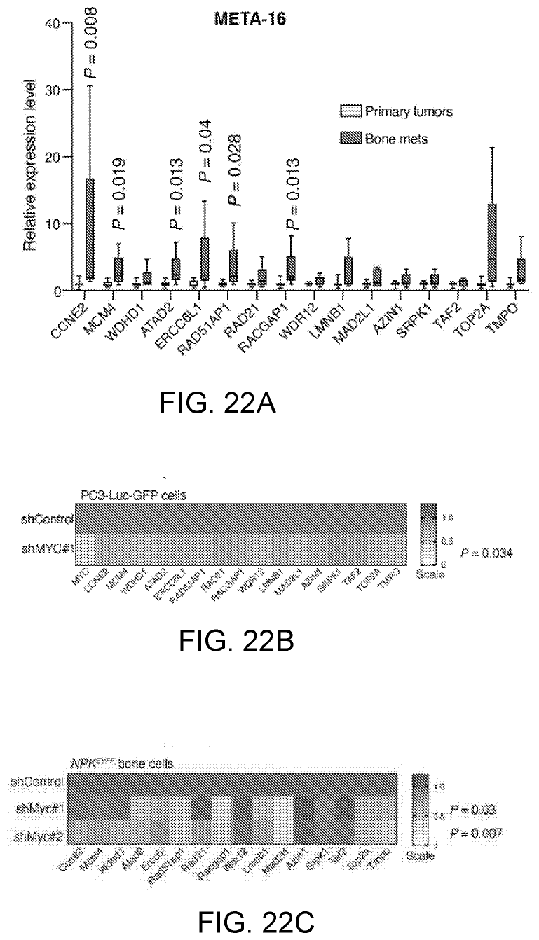

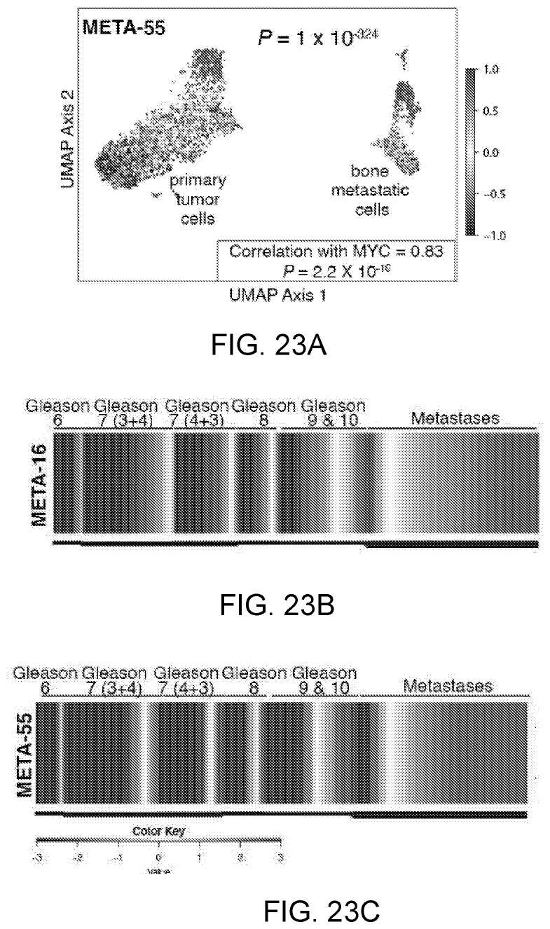

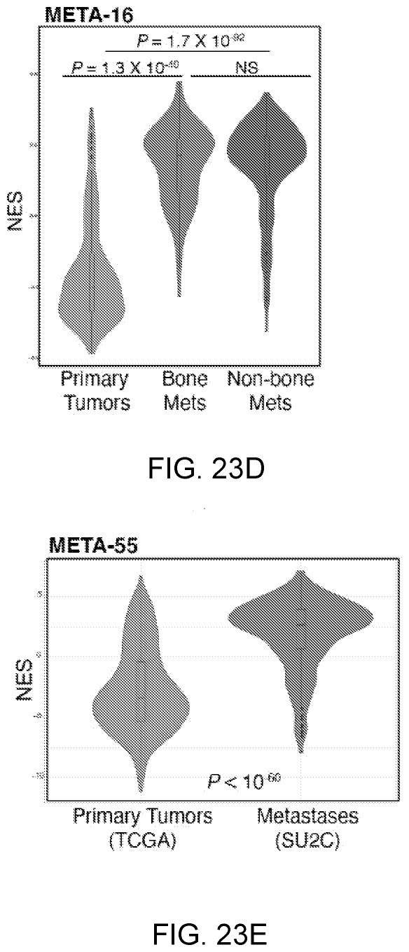

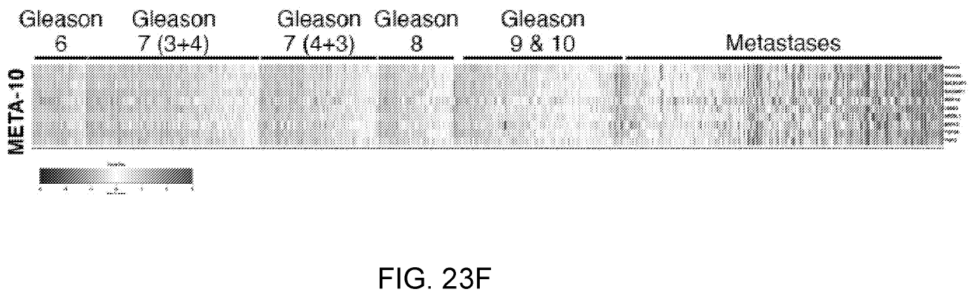

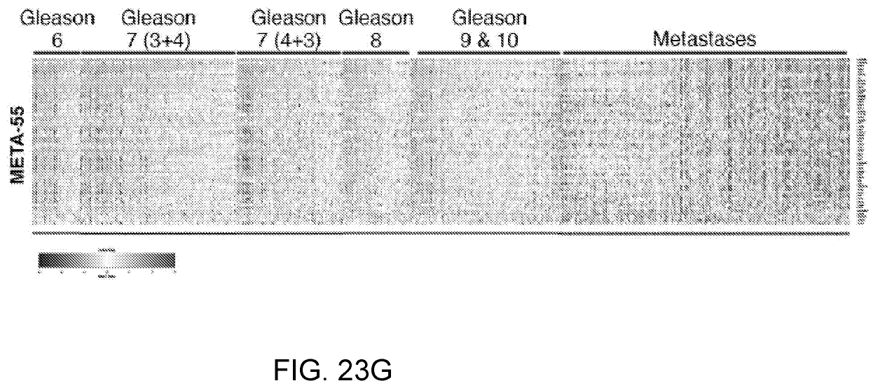

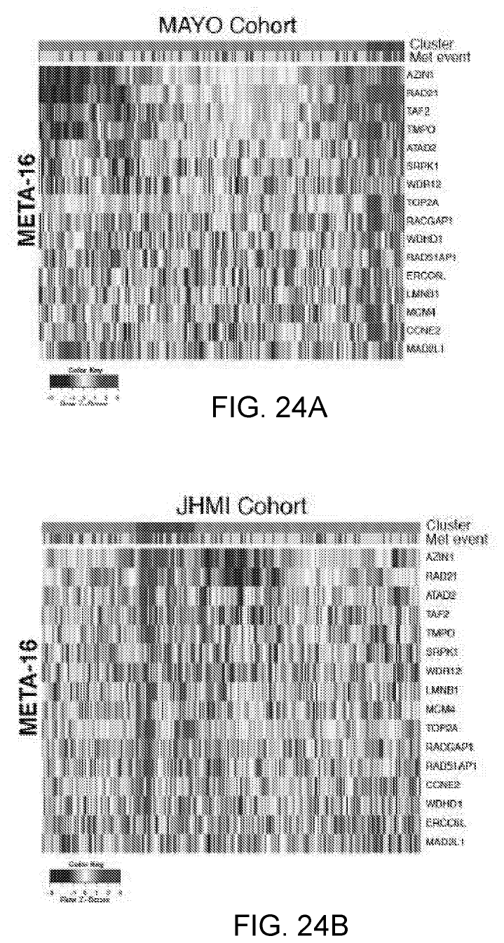

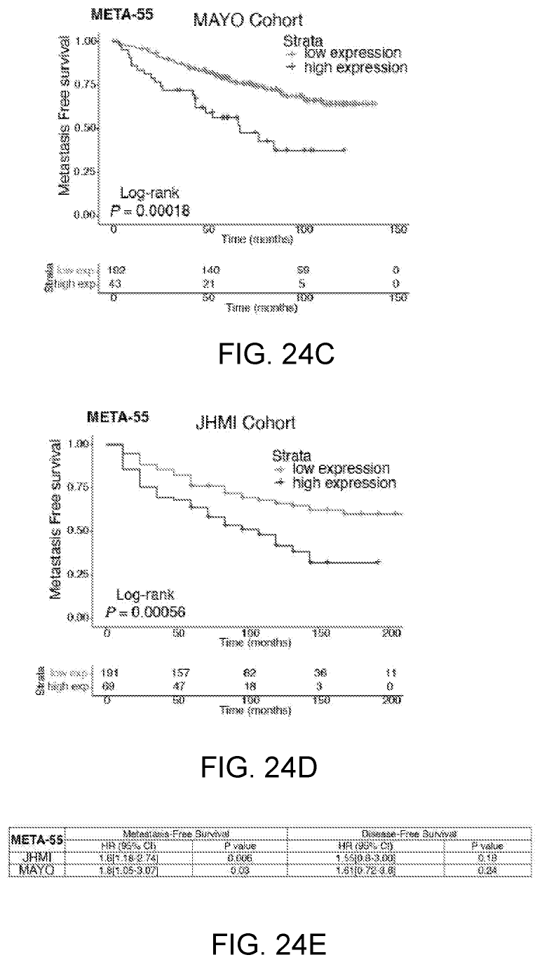

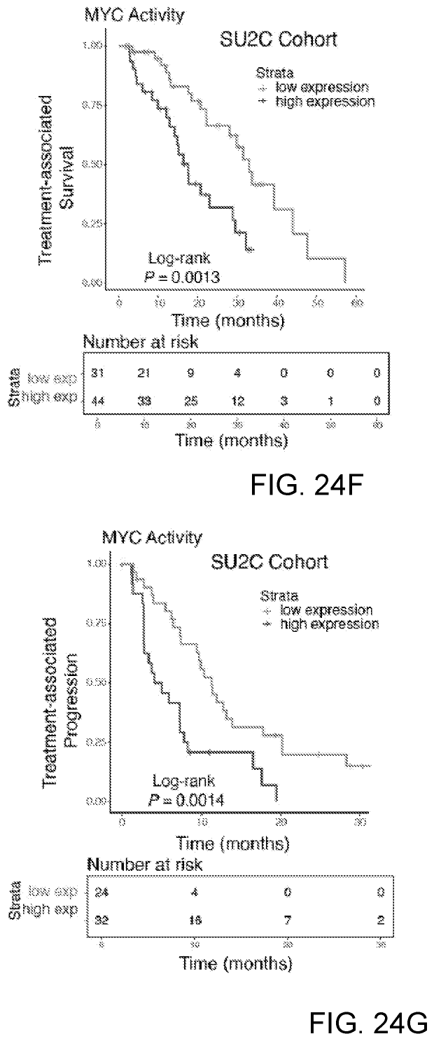

FIG. S9: Additional analyses of a MYC-correlated signature in prostate cancer metastasis (related to FIG. 6). FIG. S9A-B: GSEA using the PROMOTE-559 gene signature of MYC-correlated genes (see FIGS. 6A-B) to query the reference bone metastasis gene signatures from the NPK-CAG^{YFP} mice (in A) and the human Balk dataset (in B); NESs and p-values were estimated using 1,000 gene permutations. See also Dataset 3. FIG. S9C: Association with adverse outcome for metastasis. Each of the META-55 genes was ranked by univariable analysis of time-to-metastasis outcome using Cox proportional hazards model in the TCGA dataset (Table S2), and a cutoff at p-value<10⁻⁷ from Wald test was used to identify the 16 top-genes constituting the META-16 gene signature. FIG. S9D: Random model. To evaluate the probability that not any random group of 16 genes would be upregulated in the SU2C (n=270) versus the TCGA (n=497) cohorts (see FIG. 6E), we constructed a null model using 10,000 iterations, with the x-axis showing −log 2 p-value (from the two-sample one-tailed Welch t-test) between TCGA and SU2C comparisons and y-axis showing its probability density. The p-value of this random model thus represents an estimate of the number of times two-sample one-tailed Welch t-test p-values for a random 16 genes reached or outperformed two-sample one-tailed Welch t-test p-values for the META-16 genes.

FIG. S10: Additional analyses of the META-55 and META-16 gene signatures in prostate cancer metastases (related to FIG. 6). FIG. S10A: Single-cell heterogeneity of gene expression in primary tumors is recapitulated in bone metastases. Shown are overlapping contour plots of the cell densities of primary tumors (left) and bone metastases (right), which are superimposed on the UMAP projections of primary tumor clusters identified in FIG. 1E (colored in green, red, black and yellow). FIG. S10B: Scaled expression (DESeq2 normalized values) of the META-55 gene signature in single-cell UMAP projections of primary tumors and bone metastases (as in FIG. 3B). Shown is the correlation between META-55 expression at the single-cell level with MYC pathway activity (Spearman's rank correlation, rho=0.825, p-value=2.2×10⁻¹⁶). FIG. S10C: Heatmap representation of single-sample GSEA enrichment of the META-55 and META-16 gene signatures in primary tumors from TCGA (n=497) and metastases from SU2C (n=270) (Table S2). Colors correspond to NES. FIG. S10D: Violin plot depicting the distribution of the NESs (y-axis) which reflect activity levels of META-55 from panel C in primary tumors from TCGA (n=497) compared with metastases from SU2C (n=270). The p-value was estimated using two-sample one-tailed Welch t-test. The p-value for the random model was p-value=0.036. FIG. S10E: Heatmap representation of expression levels of each of the META-55 genes in each of the individual samples from the TCGA (n=497) and SU2C (n=270) cohorts. Gleason scores are shown for the primary tumors; metastases include all metastases in the SU2C cohort. Shown are row-scaled expression values (color). FIG. S10F: Box-plots depicting expression of the META-16 signature as Gene Set Variation Analysis (GSVA) scores. Y-axis shows META-16 GSVA scores for the prostate and different metastatic sites as indicated. The panel on the right is analyses of the SU2C cohort (Table S2). The panel on the right is a cohort from FHCRC of bone or soft tissue metastases obtained at autopsy from patients that had died from metastatic castration-resistant prostate cancer (n=138; 98 of the cases are in GEO: GSE126078).

FIG. S11: Additional validation of the META-16 gene signature in prostate cancer metastasis (related to FIG. 6). FIG. S11A: Quantitative real-time PCR (qRT-PCR) of the META-16 genes R in the CUIMC cohort of bone metastases (n=5) compared with high-Gleason grade primary prostate tumors (n=10). As indicated, ** p-value<0.01 and * p-value<0.05, estimated using a two-tailed Mann-Whitney test compared to the average of all primary tumors. FIG. S11B: Representative images of immunostaining for ATAD2 protein expression in human patient samples from benign prostate (n=2), primary tumors (n=6), brain metastases (n=6) and bone metastases (n=4) from the BERN/CUIMC cohort, depicting cases with low and high ATAD2 expression. FIG. S11C-D: Heatmaps showing the expression levels of the META-16 genes determined by qRT-PCR of following MYC silencing in human and mouse prostate cancer cells. FIG. S11C: RNA obtained from subcutaneous PC3-Luc-GFP tumors expressing shMYC#1 or the control shRNA. FIG. S11D: RNA obtained from NPK Bone cells grown in vitro. Scaled values represent ratios of expression compared to shControl, for every gene. In FIG. S11C and FIG. S11D, p-values were estimated using z-score sums of all genes by two-tailed, unpaired t-test for top heatmap and one-way ANOVA with Dunnett's multiple comparisons against shControl.

FIG. S12: Additional validation of the META-55 gene signature (related to FIG. 6). FIG. S12A-B: Heatmaps of hierarchical consensus clustering analysis used to define tumors with high (brown cluster) and low (green cluster) expression of the META-55 signatures in Mayo (n=235) and JHMI (n=260) cohorts, as indicated (Table S2). Brown vertical bars on the second from top row represent patient cases that developed distant metastasis. FIG. S12C-D: Kaplan-Meier survival analyses comparing patients with the low and high expression of META-55 as in panels FIG. S12A and FIG. S12B. The p-values were estimated using a log-rank test. FIG. S12E: Multivariable survival analysis of the META-55 gene signature in the JHMI and MAYO cohorts showing independent association with metastasis-free survival but not with prostate-cancer specific mortality (HR=hazard ratio, CI=confidence interval, p-values estimated from Cox-proportional hazards model).

FIG. 7—a mouse model of highly penetrant bone metastasis. FIG. 7A—Strategy. Tamoxifen delivery to NPK^{EYFP} mice (for Nkx3.1^{CreERT2/+}; Pten^{flox/flox}; Kras^{LSL-G12D/+}; R26R-CAG^{LSL-EYFP/}+) at 3 months induces tumor formation and lineage marking. Tumor-induced mice are monitored for 5-8 months for 581 development of metastases to bone as well as lymph node, lungs, livers and brain. FIGS. 7B-E, Histopathological analyses. FIG. 7B: Representative H&E (left) or confocal (right) images of bone metastases (spine). Shown is co-expression of YFP, with luminal cytokeratin (Ck8), basal cytokeratin (Ck5), the androgen receptor (AR), and Ki67. FIG. 7C: Representative images of prostate tumors, and metastases from lung and bone (spine, pelvis, femur, tibia, and humerus) showing ex vivo fluorescence, histology (H&E) and immunostaining for YFP. FIGS. 7D-E: Representative images of prostate tumor and metastases from androgen-intact (D) or castrated (androgen-deprived, E) NPK^{EYFP} mice showing ex vivo fluorescence, histology (H&E), and immunostaining for AR or the NEPC marker, synaptophysin. Panels B-E show representative images from 5 independent mice. Scale bars represent 0.1 cm for the ex vivo fluorescence images and 50 μm for all other images.

FIG. 8—Molecular analysis of bone metastasis from NPK^{EYFP} mice. FIGS. 8A-B: Transcriptomic analyses. FIG. 8A: Principal component analysis (PCA) of bulk RNA sequencing of primary tumors (n=15), lung metastases (n=9), and bone metastases (n=12) from androgen-intact NPK^{EYFP} mice (Table S2). The circle indicates separation of bone metastases from primary tumors and lung metastases. FIG. 8B: Conservation with human prostate cancer. Gene set enrichment analyses (GSEA) using the human bone metastasis signature based on Balk (Table S3) to query the reference mouse bone metastasis gene signature from NPK^{EYFP} mice (Table 2C). NES (normalized enrichment score) and P-values were estimated using 1,000 gene permutations. FIGS. 8C-D: Phylogenetic analysis of whole exome sequencing (WES) data. FIG. 8C: Evolutionary trees for matched trios of primary tumor, bone, and lung metastases from 5 independent mice (represented by each of the trees) were constructed by WES analyses of somatic mutations (i.e., substitutions and indels) (Table S4). The length of the lines indicates the number of mutations in each branch, and the colors indicate the mutations unique to or shared between the clones; shown are the bootstrap-derived P-values for each case using 1000 permutations. Informative copy number variations (i.e., gains in chromosome 6, “Chr 6 Gain” and deletions in chromosome 4q “Chr 4q Del”, Table S4) are shown by the red arrows. FIG. 8D: Composite phylogeny tree based on consistent evolutionary patterns across all trees in panel C. The meta-analysis P-value was calculated using one-sided Fisher's method by combining bootstrap-derived P-values from individual trees in panel C.

FIG. 9: Single-cell sequencing reveals Myc pathway activation as a cell-intrinsic feature of bone metastasis. FIGS. 9A-C: Single-cell RNA sequencing of primary tumor and bone metastasis. Uniform manifold approximation and projection (UMAP) visualization of matched primary tumor and bone samples from NPK^{EYFP} mice (Table S5). FIG. 9A: Sample of origin; black corresponds to the primary tumor sample and dark grey to the bone sample. FIG. 9B: Unsupervised clustering; colors indicate distinct clusters of cells with the relative percentages of the primary tumor and bone samples indicated. FIG. 9C: Scaled expression (DESeq2 normalized values) of YFP, Cd45, and Ck8, expression levels and AR activity levels. FIGS. 9D-E: Analysis of isolated primary tumor and bone metastatic cell clusters. FIG. 9D: Sample of origin. Black corresponds to the primary tumor cells and dark grey to the bone metastatic cells. FIG. 9E: Enrichment of the bone metastasis signature from the bulk RNA sequencing (Table S2) in bone metastatic versus primary tumor cells. The p-value was calculated by a two-sample two-tailed Welch t-test. FIGS. 9F-I: Pathway-based GSEA. FIG. 9F: GSEA comparing pathways enriched in the mouse bone metastasis signature (from bulk RNA sequencing, Table S1) with those enriched in the single-cell bone metastasis signature (Table S5). The red bar shows the location of the Hallmarks MYC pathway, which is the top-most enriched pathway across the two signatures. FIG. 9G: GSEA comparing pathways enriched in a signature from the bulk RNA sequencing comparing bone metastases and normal bone with those enriched in the single-cell bone metastasis signature (Table S5). FIG. 9H: GSEA using genes from the MYC Hallmarks pathway to query the single-cell bone metastasis signature (Table S5). FIG. 9I GSEA using genes from MYC Hallmarks pathway to query a signature based on the single-cell resident non-tumor bone cells versus the primary tumor cells (Table S5). In panels F-I, NES (normalized enrichment score) and p-values were estimated using 1,000 gene permutations. “SC” stands for single-cell.

FIG. 10: Co-activation of MYC and RAS pathways in prostate cancer metastasis. FIGS. 10A-B: Cross-species pathway analysis. GSEA comparing pathways enriched in the Balk human bone metastasis signature with, in panel A, those in the mouse single-cell bone metastasis signature (Table S5) or, in panel B, those enriched in the mouse bulk RNA bone metastasis signature. NES and p-values were estimated using 1,000 gene permutations. The red bar shows Hallmarks MYC pathway, which is the top-most enriched in both signatures. FIG. 10C: Representative immunohistochemical analyses of MYC expression in bone metastases, based on analysis of 34 mCRPC patient samples including 12 bone metastases. FIGS. 10D-G: Violin plots depicting distribution of MYC and RAS pathway activation in primary tumors and metastases in human cancer and in the NPK^{EYFP} mice. Panels D and F compare human primary tumors (TCGA, n=497) versus metastases (SU2C, n=270) (Table S3). Panels E and G compare primary tumors (n=13) and bone metastases (n=10) from the NPK^{EYFP} mice. In panels D and E, the distribution of the NESs (y-axis) represent MYC activity levels based on single-sample GSEA (see FIG. 18D). In panels F and G, the activity scores (y-axis) represent RAS pathway activity levels (based on the absolute-valued average of RAS-related genes). P-values for all violin plots were estimated using two-sample one-tailed Welch t-test. In the violin plots with embedded box plots, boxes show the 25th-75th percentile, center-lines show the median, and whiskers show the minimum-maximum values. FIG. 10H: MYC and RAS co-activation in human primary tumors and metastases. Primary tumors and metastases classified as MYC- or RAS-activated are depicted in a heatmap in red, whereas those without MYC- or RAS-activation are represented in blue. Samples were considered MYC-activated if NES scores from single-sample GSEA using MYC Hallmarks pathway were greater than the average of overall MYC activity across the cohorts. Samples were considered RAS-activated if the absolute-valued average of RAS-related genes were greater than the average of overall RAS activity across the cohorts. A black rectangle shows the samples in which MYC and RAS were co-activated. The two-tailed p-value was calculated using Fisher's exact test.

FIG. 11: Analysis of Myc function in an allograft model of bone metastasis. FIG. 11A: Strategy. Cells from a bone metastasis (femur) of NPK^{EYFP} mice were established. The original cells were passaged in Nude mouse hosts via intracardiac injection. Cells isolated from an ensuing bone metastasis, termed NPK^{EYFP} bone cells, were used herein. FIG. 11B: Western blot image showing total protein extracts from NPK^{EYFP} bone cells infected with shRNAs to silence Myc (shMyc#1, 70% inhibition; shMyc#2, 90% inhibition), or with the control shRNA (shControl). The approximate molecular weights of markers (kDa) are indicated; Actin is a control for protein loading. Shown is a representative blot from two independent experiments. Quantification of the number of metastases in bone or lung from NPK bone cells infected with shMyc#1 or shMyc#2 or shControl and introduced into Nude mouse hosts via intracardiac injection to evaluate metastasis in vivo. The p-values were estimated by one-way ANOVA with Dunnett's multiple comparisons against shControl; NS, not significant (P<0.05). In box plots, boxes show the 25th-75th percentile with the median, and whiskers show the minimum-maximum. (N=10 mice from two independent experiments). FIG. 11D: Representative ex vivo imaging of n=10 mice showing YFP fluorescence from the heart (injection site), lung, and the indicated bones from Nude mouse hosts following via intracardiac injection of NPK^{EYFP} bone cells that had been infected with shControl, shMyc#1, or shMyc#2. FIG. 11E: Representative images (n=3) of vertebrae showing ex vivo fluorescence, H&E, or immunostaining for YFP or Myc, as indicated. Scale bars represent 0.1 cm for the ex vivo fluorescence images and 50 μm for all other images.

FIG. 12: Analysis of MYC function in a new GEMM. FIGS. 12A-E: Comparative characteristics of the tumor and metastatic phenotypes of NP^{EYFP} (n=35), NPK^{EYFP} (n=23), NPK^{EYFP} (n=106) and NPKM^{EYFP} (n=10) mice. FIG. 12A: Representative bright field and ex vivo fluorescence images of prostate, lung, and bone (spine). Scale bars represent 0.1 cm. FIG. 12B: Dot-plots showing tumor weights. Center-lines show the mean, error bars depict standard deviation; P-value is shown for one-way ANOVA with Dunnett's multiple comparison test of NPM^{EYFP} and NP^{EYFP} mice. Kaplan-Meier curves showing overall survival; p-value calculated using a two-tailed log-rank test. FIG. 12C: percent survival over time in months. FIG. 12D: Bar graphs showing the percentage of mice with metastasis to lung and bone. FIGS. 12E-F: Violin plots depicting the distribution of Myc (E) and Ras (F) pathway activity levels in primary tumors of NPM^{EYFP} (n=3) and NPK^{EYFP} (n=13) mice and bone metastases of NPK^{EYFP} mice (n=10). Myc activity is based on single-sample GSEA and Ras pathway activity is based on the absolute-valued average of RAS-related genes. The p-values were estimated using two-sample one-tailed Welch t-test. In the violin plots with embedded box plots, boxes show the 25th-75th percentile, center-lines show the median, and whiskers show the minimum-maximum values.

FIG. 13: META-16 is correlated with MYC and RAS pathway activation and enriched in prostate cancer metastasis. FIGS. 13A-B: Discovery of the META-16 gene signature. FIG. 13A: Step 1, genome-wide Spearman correlation to MYC expression in PROMOTE cohort (which includes 55 bone metastases), identified 559 (PROMOTE-559) positively correlated genes (FDR p-value<0.0001, Spearman rank correlation coefficient rho plotted in the x-axis. FIG. 13B: Step 2, GSEA using PROMOTE-559 to query the mouse (NPK^{EYFP}) and human (Balk) bone metastasis signatures (see Extended FIG. 7a,b). In panel b, the leading-edge genes from mouse are projected on the y-axis and from human on the z-axis. In panel B, the leading-edge genes from mouse are projected on the y-axis and from human on the z-axis. These analyses identified 55 genes (META-55, highlighted in red in panel FIG. 13B: Step 3, ranking of META-55 according to metastasis-free survival identified 16 genes (META-16, shown by name in panel B. FIGS. 13C-D: UMAP projection of single-cell RNA sequencing showing the primary tumor and bone metastatic cells (see FIG. 9D). Panel C shows enrichment of the MYC pathway and panel D expression of META-16. Scaled DESeq2 normalized values are depicted. The correlation between META-16 expression and MYC pathway activity was estimated using Spearman's rank correlation. FIG. 13E: GSEA using META-16 to query single-cell bone metastasis signature (Table S5); NES and P-value were estimated using 1,000 gene permutations. FIG. 13F: Violin plot depicting the distribution of the NESs (y-axis) which reflect activity levels of META-16 (as in FIG. 22B) in primary tumors from TCGA (n=497) compared with metastases from SU2C (n=270) (Table S3). The p-value was estimated using two-sample one-tailed Welch t-test. In the violin plots with embedded box plots, boxes show the 25th-75th percentile, center-lines show the median, and whiskers show the minimum-maximum values. FIG. 13G: Heatmap representation of individual expression levels of META-16 genes patient samples from the TCGA and SU2C cohorts. Gleason scores are shown for the primary tumors; metastases include all metastases in the SU2C cohort. Shown are row-scaled expression values (indicated by the different levels of shading).

FIG. 14: The META-16 signature is associated with metastasis-free and treatment-associated survival. FIGS. 14A-C: Association of META-16 with time to metastasis. FIGS. 14A-B: Kaplan-Meier survival analyses comparing patients with low and high combined expression of META-16 in the MAYO (n=235) and JHMI (n=260) cohorts (see FIG. 24A-B). The p-values were estimated using a log-rank test. FIG. 14C: Multivariable survival analysis of META-16 with the JHMI and MAYO cohorts. HR=hazard ratio, CI=confidence interval, p-values estimated from Cox proportional hazards model. FIGS. 14D-E: Kaplan-Meier survival analyses comparing patients from the SU2C cohort with low and high combined expression of META-16 showing treatment-associated survival (i.e., time from the start of treatment with androgen receptor signaling inhibitor (ARSi) therapy, to death or last follow-up, n=75 patients) or treatment-associated disease progression (i.e., time on treatment with ARSIs, n=56). The p-values were estimated using a log-rank test.

FIG. 15: Analysis of MYC function in a new GEMM. FIGS. 15A-E: Comparative characteristics of the tumor and metastatic phenotypes of NP^{EYFP}, NPK^{EYFP} (without bone metastases) and NPK^{EYFP} (with bone metastases) mice. FIG. 15A: Representative bright field and ex vivo fluorescence images of prostate, lung, and bone. Scale bars represent 0.1 cm. FIG. 15B: Dot-plots showing tumor weights. FIG. 15C: percent survival over time in months. FIG. 15D: Comparison of tumor weight. FIG. 15E: comparison of number of metastases with and without bone metastases in lung. FIG. 15F: comparison of number of metastases with and without bone metastases in liver. FIG. 15G: comparison of number of metastases with and without bone metastases in brain. FIG. 15H: bar graph showing number of bone metastases in all, spine, femur, pelvis, tibia, humerus and other over time (in months). FIG. 15I: bar graph showing percentage of mice with bone micromets over time (months). FIG. 15J: bar graph showing percentage of mice with micromets/mets in bone and lung.

FIG. 16: Comparison of expression of activity levels of AR and NEPC for Castrated and Non-Castrated Mice. FIG. 16A: Comparison of percent survival over months. FIGS. 16B-C: Comparison of tumor weight and bone metastases, respectively, for castrated versus non-castrated. FIG. 16D: Percentage of mice with metastases to lymph node, lung, liver and bone. FIGS. 16E-F: Relative AR and NEPC activity, respectively, for primary tumor, lung and bone. FIG. 16G: Running enrichment scores as a function of gene list index.

FIG. 17: Cooperation of MYC and RAS Activity in Mouse and Human Cohorts. FIG. 17A: Copy number variation for Kras, Cdkn2a/b, Myc for five mice. FIGS. 17B-C: Comparison of MYC and RAS genomic alterations in primary tumors and metastasis across human cohorts. FIGS. 17D-E: Mys and Ras activity, respectively, of primary tumors, bone metastases and lung metastases. FIGS. 17F-G: Violin plots depicting distribution of MYC and RAS pathway activation in primary tumors and metastases in bone and non-bone metastases. FIGS. 17H-J: Correlation and co-activation of MYC and RAS activities across primary tumors and metastasis in mouse and human cohorts.

FIG. 18: Activation of MYC in bone metastasis across various mouse and human cohorts. FIG. 18A: Cross-species GSEA comparing pathways enriched in the mouse single-cell bone metastasis signature with those enriched in a human signature comprised of primary tumors and bone biopsies. FIG. 18B: Stouffer integration to identify pathways significantly enriched and conserved among all three mouse and human signatures. FIG. 18C: Activation of MYC pathways in mouse and human signatures of bone metastasis. FIG. 18D: Activation of MYC in primary tumors and metastasis in human cohorts.

FIG. 19: NPK^{EYFP} cells used in intracardiac injection experiments. FIG. 19A: Colony formation analyses. Right, representative images of crystal violet-stained colonies. Left, quantification; p-values were estimated by one-way ANOVA with Dunnett's multiple comparisons against shControl. In vitro assays were repeated 3 times in triplicate; a representative experiment is shown. FIG. 19B: Representative H&E sections of primary tumors in lungs and bones (spine) of mice injected with NP^{GFP} and NPK^{EYFP} cells.

FIG. 20: A mouse model of highly penetrant bone metastasis. FIG. 20A—Strategy. Human PC3 cells were infected with Luciferase-GFP vecot, treated with control or MYC-targeting shRNAs and implanted into the tibiae of SCID mice. FIG. 20B: Western blot image confirming MYC knockdown in PC3 cells. FIG. 20C: MYC staining by immunohistochemistry on subcutaneous tumors confirming MYC knockdown. FIG. 20D: Colony formation analyses. Right, representative images of crystal violet-stained colonies. Left, quantification; p-values were estimated by one-way ANOVA with Dunnett's multiple comparisons against shControl. In vitro assays were repeated 3 times in triplicate; a representative experiment is shown. FIG. 20E: Representative H&E (left) or confocal (right) images of bone metastases (spine). Shown is co-expression of YFP, with luminal cytokeratin (Ck8), basal cytokeratin (Ck5), the androgen receptor (AR), and Ki67. FIG. 20E: Representative images of mice infected with shMYC#1 or shMYC#2 or shControl.tumors. FIG. 20F: Plot of total flux as a function of time (days) for shMYC#1 or shMYC#2 or shControl.tumors. FIG. 20G: Representative images from the time of sacrifice of tibias implanted with the PC3-Luc-GFP cells infected with shMYC#1 or shMYC#2 or shControl. Shown are ex vivo imaging of YFP fluorescence, to visualize the tumor, and corresponding micro-computed tomography (CT) images, to show areas of osteolysis as is typical of PC3 tumors in bone. Also shown are representative H&E and immunostaining for YFP. Scale bars represent 0.1 cm for the fluorescent images and 50 μm for histological images (H&E and IHC).

FIG. 21: Discovery of META-16 gene signature of bone metastasis. FIGS. 21A-B: Comparative analysis of META-559 gene set to mouse and human signatures of bone metastasis. FIG. 21C: Distribution of ability to predict time to bone metastasis for candidate genes, with META-16 genes indicated. FIG. 21D: Random model, showing non-random ability of the META-16 candidate genes to predict time to metastasis.

FIG. 22: Expression of Gene Signature META-16. FIG. 22A: Relative expression level of the 16 genes of META-16 with regard to primary tumors and bone metastases. FIGS. 22B-C: Heatmaps showing the expression levels of the META-16 genes determined by qRT-PCR following MYC silencing in human PC3 (B) and mouse NPK^{EYFP} (C) prostate cancer cells.

FIG. 23: Comparative analysis of candidate gene sets in primary tumors and bone and non-bone metastasis in mouse and human cohorts. FIG. 23A: Bone metastasis signature from the bulk RNA sequencing in bone metastatic versus primary tumor cells. FIG. 23B: Heatmap representation of single-sample GSEA enrichment of the META-16 gene signature in primary tumors from TCGA (n=497) and metastases from SU2C (n=270). Colors correspond to NES. FIG. 23C: Heatmap representation of single-sample GSEA enrichment of the META-55 gene signature in primary tumors from TCGA (n=497) and metastases from SU2C (n=270). Colors correspond to NES. FIG. 23D: Violin plot depicting the distribution of the NESs (y-axis) which reflect activity levels of and META-16 in primary tumors, bone metastases and non-bone metastases. FIG. 23E: Violin plot depicting the distribution of the NESs (y-axis) which reflect activity levels of and META-55 in primary tumors from TCGA (n=497) compared with metastases from SU2C (n=270). FIGS. 23F-G: Heatmap representation of expression levels of each of the META-10 and META-55 genes respectively in each of the individual samples from the primary tumors and metastatic samples, obtained from TCGA and SU2C patient cohorts, respectively. Gleason scores are shown for the primary tumors. Shown are row-scaled expression values (color).

FIG. 24: Clustering of patients in MAYO and JHMI cohorts based on the expression levels of META-16. FIGS. 24A-B: Heatmaps of hierarchical consensus clustering analysis used to define tumors with high and low expression of the META-16 signatures in Mayo (n=235) and JHMI (n=260) cohorts. FIGS. 24C-D: Plots of metastasis-free survival versus time for the Mayo and JHMI cohorts, respectively. FIG. 24E: Adjusted Cox proportional hazards model, based on expression levels of META-55 genes, demonstrating their association to metastatic and disease-free survival. FIGS. 24F-G: Plots of treatment-associated survival versus time for the SU2C cohort.

### DETAILED DESCRIPTION

In certain embodiments, the present technology is directed to a gene signature that is capable of stratifying indolent and metastatic patients in the clinical management of cancer. In various embodiments, the gene signature can include no more than 10 genes, no more than 16 genes, no more than 50 genes or no more than 55 genes.

The present disclosure provides, in certain embodiments, novel biomarkers that can distinguish indolent or non-metastatic cancer from high-risk or metastatic primary tumors, which are associated with an aggressive clinical course. In particular for prostate cancer, many newly diagnosed patients present with indolent disease that will not disseminate beyond the prostate and can be managed by active surveillance or local therapy, without the need for more invasive therapies.

At present, there are no biomarkers that accurately predict which primary tumors are likely to metastasize and progress to lethality versus those that will remain localized to the prostate. Clinicians and researchers alike increasingly recognize, and would benefit from, the need to identify molecular markers with better prognostic value.

In certain embodiments, the present technology is directed to novel gene signatures that can be used as biomarkers to predict the future development, e.g., the development of bone metastasis of prostate cancer, and to help make clinical decisions in the management of cancer patients.

In certain embodiments, a 55-gene signature, 16-gene signature, or a subset thereof, to sub-stratify indolent and metastatic patients with different Gleason scores can be used in the clinical management of prostate cancer.

The present signatures of metastatic cancer, including prostate cancer or a subset thereof, can be used for prognosis of patients diagnosed with cancer, whether localized or metastatic. This can be used in, in certain embodiments, a prognostic test. In certain embodiments, the present technology is directed to such a prognostic test, including but not limited to a biomarker PCR-based, RNA-seq based or NanoString based kit, that can stratify patients at risk of developing metastasis. In certain embodiments, a prognostic assay kit according to the present technology permits RNA extraction to quantify mRNA levels of the present gene signatures. In certain embodiments, the sample collection includes separating a part of the tissue specimen for RNA extraction and subsequent analysis.

In certain embodiments, the technology herein is directed to a method for diagnosing metastasis, or of assessing the risk of metastasis; or of treating a subject with metastatic cancer or an increased risk of cancer metastasis. As used herein, “metastasis” (occasionally referred to herein abbreviated as, “mets”) means the development of secondary malignant growths at a distance from a primary site of cancer. As used herein, “treat” and “treatment” mean any amelioration or lessening of the symptoms or biological condition (for example, slowing the growth of cancer cells), and includes treatment that fall short of a full cure.

In various embodiments, the methods herein include taking steps (either diagnosing, assessing risk or treating) when the expression level of at least one gene discussed herein increases by at least 10%, at least 15%, at least 20%, at least 30%, at least 50%, about 20 to about 90%, or about 20 to about 75% compared to the reference level or its expression level in the control sample.

In various embodiments, the cancer can be any cancer that affects mammals; including but not limited to cancer of the breast, digestive/gastrointestinal systems, endocrine and neuroendocrine systems, eye, genitourinary system, germ cell, gynecologic, head and neck, hematologic/blood, musculoskeletal systems, neurologic system, respiratory system, thoracic system, or skin. These include, e.g., cancer of the lung, breast, pancreas, prostate, liver or colorectal system. Similarly, in various embodiments, the metastasis herein can be metastasis of any other part of the body other than the location of the original cancer. In certain embodiments, the methods herein are useful for treatment of prostate cancer that has metastasized to any other part of the body, including the bone, e.g., osteolytic metastasis.

In various embodiments, the methods herein include obtaining a sample from a patient; the sample can be from any part of the patient's body that can provide cells useful for the assessments herein; including, e.g., blood, plasma, serum, or a sample of tissue from a tumor. In certain embodiments, a control sample is provided, where the control sample is from a subject who is healthy or has a metastasis-free cancer. As used herein, “healthy” means having no signs of cancer—that is, no signs of abnormal growth of cells or tumors. As used herein, “metastasis-free” means having cancer cells that have not spread to another part of the body from their primary (original) location.

In certain embodiments, the methods herein involve measuring the expression level of one or more genes; in various embodiments, the expression level can determined by, e.g., assaying an mRNA level, by polymerase chain reaction (PCR), by RNA sequencing (RNA-seq), by assaying a protein level, or through nCounter technology.

In certain embodiments, a kit herein can be a valuable tool for clinical decision making such as identifying patients in need of more aggressive therapy to prevent metastatic disease outcome, or as a novel end-point in clinical trials evaluating the therapeutic value of novel drugs or drug combinations. In various embodiments, a kit herein can comprise any of the following: equipment adapted to gather the sample from the subject (for example, a biopsy tool or syringe); to store the sample in sterile conditions (for example, medical container); to measure the expression level of any of the genes discussed herein in the sample (for example, the methods for determining expression levels discussed above); to compare the expression level with the known control (for example, through a stored electronic database or processor); to diagnose the presence of metastatic cancer or the increased risk of cancer metastasis (for example, through a stored electronic database or processor with a known standard or threshold); and to determine the therapy for the subject based on the results of the diagnosis (for example, based on a library or stored database of recommended actions and predicted outcomes). In certain embodiments, an additional step of treatment can be involved; this could include chemotherapy, radiation therapy, surgery or any other treatment designed to ameliorate the condition of the patient.

### Mouse Models

Genetically engineered mouse models of prostate cancer have been used herein to study the progression of this disease, and cross-species analyses have been used to gain insights into the molecular basis of human prostate cancer progression. In certain embodiments, by combining sequential genetic alterations, different unique mouse models have been developed therein; these models progressively develop pre-malignant, indolent and bone-metastatic disease.

Given the lack of understanding in the genesis and natural history of prostate cancer, in part due to the absence of relevant biologic models of the disease, the mouse models discussed herein have been found to be able to provide unique and powerful tools to gain functional, biological and molecular insight into the metastatic disease.

In particular, in certain embodiments, the inducible Nkx3.1^{CreERT2}; Pten^{flox/flox}; Kras^{LSL-G12D} strain described herein (termed “NPK” mice) is unique in its ability to generate metastasis with high penetrance, permitting the study of the molecular basis of bone metastasis in particular, which is the most frequent metastatic site in human prostate cancer. In particular, the present studies have shown that NPK mice tend to develop prostate cancer with a high penetrance of metastasis to bone, permitting detection and tracking of bone metastasis in vivo and ex vivo. Transcriptomic and whole-exome analyses of bone metastasis from the mice, and cross species analyses of mouse bone metastasis and human prostate cancer have, in certain embodiments, revealed distinct molecular profiles conserved between human and mouse, and specific patterns of sub-clonal branching from the primary tumor. Integrating bulk and single-cell transcriptomic data from mouse and human datasets, as described herein, with functional studies in vivo have been shown to unravel a unique MYC/RAS co-activation signature associated to prostate cancer metastasis. With this information, a gene structure with prognostic value for time to metastasis in human patients undergoing AR therapy can be developed, that is predictive of treatment response across clinical cohorts; thus uncovering conserved mechanisms of metastasis with potential translational significance.

The biological and molecular features of bone metastasis of metastatic prostate cancers have been investigated herein, including biomarkers with direct clinical applicability. As the NPK mice provide a unique model of bone metastatic prostate cancer, bioinformatic approaches have been used to identify gene signatures for bone metastasis. This began by using cross-species differential gene expression analysis of bone metastasis and primary tumors in the mouse models herein, as well as in publicly available human prostate cancer datasets.

Gene Set Enrichment Analysis (GSEA) using Kolmogorov-Smirnov statistics of signaling pathways in the Hallmarks database identified MYC signaling as a conserved pathway enriched in bone metastasis. To identify the genes that could mediate or cooperate with MYC in bone metastasis, the PROMOTE dataset was used herein to perform genome-wide correlation of MYC mRNA levels, followed by studying how many of these MYC-correlated genes were also upregulated in bone metastasis versus primary tumors in the mouse model and in a human training dataset. This analysis led to, in certain embodiments, the identification of gene signatures associated with time to metastasis in primary prostate cancer and response to anti-androgen treatment in metastatic disease.

In certain embodiments, a gene signature herein comprises 55 genes that have been found to be significantly upregulated in bone metastasis, and their expression has been found to correlate significantly with MYC. Upon further investigation, this gene signature has been confirmed to be upregulated in two other human prostate cancer datasets used for validation purposes [SU2C/TCGA, FHCRC], two-sample two-tailed Welch t-test p=0.0001 and p=0.05, respectively. Moreover, in certain embodiments, this 55-gene signature was also present in a subset of primary tumors of different Gleason score. Therefore, in certain embodiments, the gene signatures can be used to identify primary tumors that could have the potential to metastasize.

In certain embodiments, the power of the 55-gene signature was validated in predicting metastasis outcome in localized, primary human prostate cancer samples. To this end, the expression of this signature in the TCGA dataset of primary tumors was analyzed in relation to time-to-metastasis outcome. Kaplan-Meier analysis followed by log-rank statistics showed a significant association of the signature with time-to-metastasis (p=0.00019) outcome, but not to other prognostic markers such as biochemical recurrence, disease-specific survival, or overall survival, indicating the power of this gene signature to specifically relate to metastatic events.

### Example 1

A Highly-Penetrant Mouse Model of Prostate Cancer Bone Metastasis Conserved with Human Metastasis Progression

As discussed above, although the primary site of prostate cancer metastasis is bone, it has proven challenging to model bone metastasis in vivo. Here, a genetically engineered mouse model (GEMM) of prostate cancer was developed based on co-activation of P13 Kinase and RAS signaling (aka NPK-CAG^{YFP} mice) that metastasizes to bone with high penetrance, thereby permitting phenotypic and molecular analyses in the context of the native tumor environment in vivo. Lineage-tracing permits direct visualization of bone metastases, while histological and single-cell sequencing analyses enable phenotypic and molecular comparison with primary tumors and metastases to other sites.

It has been shown herein that bone metastases arise from a distinct sub-clone of the primary tumor, and have distinct transcriptomic characteristics compared with metastases to other sites. Cross species analysis of a mouse bone metastasis signature revealed conservation with human prostate cancer metastasis, and identified MYC as a key driver of metastasis. MYC protein is expressed in human bone metastasis, and is necessary for tumor growth in bone as well as metastasis to bone in human xenograft and mouse allograft models, respectively.

Notably, a MYC-correlated gene signature, META-16, was identified; and in certain embodiments herein, this is prognostic for time to metastasis in prostate tumors. This constitutes a unique model of bone metastasis that can impact prognosis and treatment of metastatic prostate cancer. In certain embodiments, META-16 can be used in prognostic tests for identifying patients with localized prostate cancer that have increased risk of developing metastasis.

As stated above, current in vivo models based on prostate cancer cells implanted in bone do not fully capture the metastatic processes as it occurs during tumor evolution and progression in the context of the nature microenvironment. Moreover, the known mouse models addressing de novo bone metastasis described in the literature have all exhibited relatively low penetrance, and thus have limited utility for molecular or preclinical investigations.

These challenges have now been overcome through the present development of a genetically engineered mouse model (GEMM) of lethal prostate cancer that develops bone metastasis with high penetrance (44%), thereby permitting investigations of the biological processes and molecular mechanisms associated with de novo bone metastasis in vivo. Cross species analysis comparing a mouse gene signature of bone metastasis with comparable signatures of human prostate cancer metastasis reveal MYC as a key driver of prostate cancer metastasis, and permitted identification and development of the META-16 gene signature.

Results

### A. A Highly Penetrant Mouse Model of Bone Metastasis

A mouse model of prostate cancer bone metastasis was generated using a second generation conditionally-activatable reporter allele with enhanced fluorescence (R26R-CAG-^{LSL-EYFP/+}) to improve detection of prostate tumors and their metastases (FIG. 1A). Specifically, an enhanced fluorescence reporter allele, the R26R-CAG-^{LSL-EYFP/+} allele, was crossed with NPK mice (for Nkx3.1^{CreERT2}+, Pten^{flox/flox}; Kras^{LSL-G12D/+}), since it has been shown that NPK mice develop lethal prostate cancer with highly penetrant metastasis including disseminated tumor cells in bone.

The Nkx3.1^{CreERT2/+} allele utilizes an inducible Cre expressed under the control of the promoter of the Nkx3.1 homeobox gene to achieve temporal- and spatial-regulation of Cre-mediated recombination in a luminal cell of origin of prostate cancer, while at the same time introducing heterozygosity for Nkx3.1 as frequently occurs in human prostate cancer. Conditional deletion of Pten (Pten^{flox/flox}) with simultaneous activation of mutant K-Ras (Kras^{LSL-G12D/+}) models co-activation of PI3 Kinase and RAS signaling, which is prevalent in lethal prostate cancers in humans, wherein PTEN deletions are common while KRAS mutations are part of the long tail of low-incidence, significantly mutated genes in prostate cancer.

The resulting NPK-CAG^{YFP} mice (for Nkx3.1^{CreERT2/+}; Pten^{flox/flox}; Kras^{LSL-G12D/+}; R26R-CAG-^{LSL-EYFP} display lethal prostate cancer (median survival=4.7 months) with high grade aggressive histopathology in the tumor-induced (n=106) but not in control un-induced (n=3) NPK-CAG^{YFP} mice (FIGS. 1A-B, S1A-B, S2A-G, Table S1), similar to the original NPK mice and distinct from the non-lethal, non-metastatic NP-CAG^{YFP} mice (for Nkx3.1^{CreERT2/+}; Pten^{flox/flox}; R26R-CAG-^{LSL-EYFP/+}) (n=25, Table S1).

Lineage tracing revealed the highly penetrant, widespread metastatic phenotype of the NPK-CAG^{YFP} mice. This was evident by ex vivo fluorescence as well as immunohistochemical staining for YFP in primary tumors and metastatic sites specifically in the tumor-induced NPK-CAG^{YFP} mice and not in the un-induced NPK-CAG^{YFP} mice nor in the non-metastatic NP-CAG^{YFP} mice (FIGS. 1A-C, SIB, Table S1).

Most notably, analysis of a large cohort of NPK-CAG^{YFP} mice revealed that 44% (n=47/106) display fluorescence in the bones, indicative of bone metastasis (FIGS. 1B, S1B Table S1). Ex vivo fluorescence was evident in the spine (n=32/47), pelvis (n=18/47), femur (n=22/47), tibia (n=9/47), and humerus (n=9/47) (FIG. 1B, Table Si), which are frequent sites of bone metastasis in human prostate cancer. Histopathological analysis confirmed the presence of metastatic lesions in the bone, which have similar histopathology as the primary prostate tumors (FIGS. 1B, S1A-B). Notably, these metastatic cells express YFP protein (FIGS. 1B, C, S1B), indicating that they originated from lineage-marked Nkx3.1-expressing prostatic epithelial cells. Further, these metastatic cells in bone express several markers that are expressed in primary tumors, including luminal cell cytokeratin (Ck8) and androgen receptor (AR), and are highly proliferative, as evident by immunostaining for Ki67 (FIGS. 1C, Fig. S1B). Notably, the histopathology of the mouse bone metastases resembles osteoblastic lesions, while molecular analyses of their expression profiles reveals strong conservation with a human osteoblastic prostate cancer signature (FIG. 1B, Fig. S1B, S2D, Dataset 1).

To investigate the cell-intrinsic molecular phenotype of the bone metastatic cells compared with the primary tumor cells, single-cell RNA sequencing was performed using a 10× Genomics Chromium platform and Illumina NovaSeq (Dataset 2). Two matched samples were analyzed, one of which was obtained from the primary prostate tumor and the other from the interior of two bones (spine and femur) in which we had detected ex vivo fluorescence (i.e., bone metastasis). As visualized using uniform manifold approximation and projection (UMAP), cells from the primary tumor sample (black) separated into a major group (95% of the cells, hereafter called the primary tumor cluster) and a second smaller group (5% of the cells), while the cells from the bone sample (dark grey) separated into an elongated major group (88% of the cells) and a dense smaller group (12% of the cells) (FIG. 1D).

Unsupervised clustering of the combined samples revealed the presence of multiple sub-clusters within each sample (i.e., the primary tumor and bone samples) (FIG. 1E). The larger group of bone cells, projected further from the primary tumor, was comprised of a mixture of untransformed, CD45+ cells (i.e., non-metastatic bone cells), while the smaller group of bone cells, projected closer to the primary tumor, was comprised of transformed, YFP+ cells (i.e., bone metastatic cells) (FIGS. 1E-F). Notably, the primary tumor cell and bone metastatic cell clusters were highly enriched for expression of CK8 and AR, as well as YFP (two-sample two-tailed Welch t-test p-values<10⁻¹⁸, FIG. 1F), similar to the histopathological analyses of the bone metastases (FIGS. 1B-C). Taken together, these phenotypic and molecular analyses demonstrate that lineage marked cells in the bone of NPK-CAG^{YFP} mice had cell-intrinsic features of primary prostate tumor cells and therefore represent bona fide bone metastases.

The next consideration was the factors that might distinguish the nearly half of the NPK-CAG^{YFP} mice that develop bone metastases (n=47/106) from their counterparts that develop metastases to other sites but not overtly to bone (n=59/106) (Table S1). Consistent with the inherent androgen insensitivity of NPK tumors, androgen deprivation following surgical castration did not influence the incidence of bone metastases in the NPK-CAG^{YFP} mice, nor did it have a significant effect on overall survival, tumor weight or the molecular phenotype of the tumors or metastases (Fig. S2E-G). Furthermore, no significant differences were observed in either the histopathology of primary tumors from NPK-CAG^{YFP} mice with or without bone metastases; or in tumor weight or other non-tumor features, such as coat color (FIGS. S1A, S2A-C, Table S1).

In fact, the most notable distinction of the NPK-CAG^{YFP} mice with bone metastases, compared with those without bone metastases, was their significantly augmented metastatic phenotype overall (FIGS. S2A-C, Table S1). The occurrence of bone metastasis in NPK-CAG^{YFP} mice was significantly correlated with a “high metastatic” phenotype, as assessed based on the number of lung metastases and the occurrence of metastases to liver and brain (two-sided Fisher's exact test, p-value<0.0001, Table S1). In particular, NPK-CAG^{YFP} mice without bone metastasis had an average of 25+ lung metastases, and rare if any metastases to liver or brain (i.e., “low metastatic” phenotype) whereas those with bone metastasis had an average of 80+ lung metastases, 40+ liver metastases, and 1 or more brain metastases (i.e., “high metastatic” phenotype) (FIG. S2C, Table S1). Additionally, NPK-CAG^{YFP} mice with bone metastases had a modest but statistically significant decrease in survival compared to those without bone metastases (log-rank p-value=0.032, FIG. S2A, Table S1) and were less likely to die of bladder occlusion (two-tailed Mann-Whitney test, p-value=0.024, FIG. S2B, Table S1).

To gain molecular insights regarding the distinct metastatic potential of these NPK-CAG^{YFP} mice, RNA sequencing analyses were performed to compare expression profiles of primary tumors from mice that did (n=10) or did not (n=4) have bone metastases, which identified 299 differentially expressed genes (128 up-regulated and 172 down-regulated genes, two-sample two-tailed Welch t-test p-value<0.001; FIG. S3A, Dataset 1). Gene set enrichment analyses (GSEA) on this signature using the C2 (i.e., KEGG, Reactome and BioCarta) and Hallmarks pathway databases from MSigDB revealed significant enrichment of key pathways associated with metastasis, including positive enrichment of MYC and DNA repair pathways, and negative enrichment of the TNFA signaling pathway (FIG. S3B, Dataset 3). Taken together, these biological, histopathological, and molecular analyses demonstrate that osteoblastic bone metastases arise in NPK-CAG^{YFP} mice that have an enhanced metastatic phenotype.

B. Bone Metastases have Distinct Sub-Clonal Origin and Transcriptomic Profiles

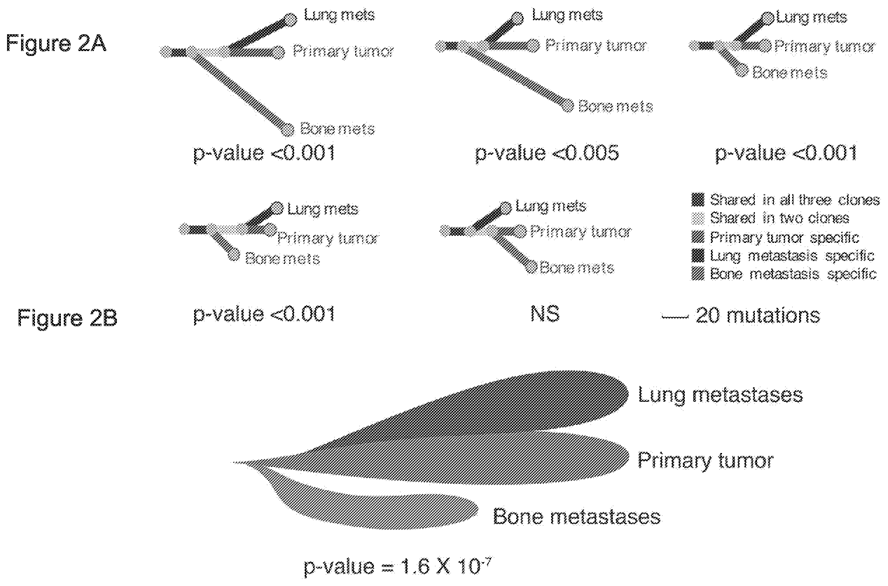

The high penetrance of bone metastases in NPK-CAG^{YFP} mice permitted in depth molecular analyses of the bone metastases compared with primary tumors and metastases to other tissue sites (FIG. 1A). To understand the clonal origin of bone metastases, whole exome sequencing (WES) was performed on five “high metastatic” NPK-CAG^{YFP} mice using genomic DNA isolated from matched sets of primary tumors, bone metastases, and lung metastases, as well as normal DNA obtained from the tails of these mice (FIG. 2). These analyses identified 372 somatic mutations (i.e., substitutions and indels) in the five mice with a variant allele frequency more than 5% (Dataset 4).

Similar to reports of whole exome sequence analysis of metastasis of other GEMMs reported in the art, significant somatic protein-changing recurrent point mutations (i.e., substitutions or indels) or known tumor suppressors or oncogenes in the primary tumor or metastases were not identified (Dataset 4). Nonetheless, the exome captured mutations (including synonymous, UTR, and intronic) allowed reconstruction of the relation between the dominant clones in the different samples within each matched set (FIG. 2A). In principle, for a set of four samples (i.e., normal, primary and two metastasis) there are three potential phylogenetic topologies, reflecting three potential clonal histories: (i) metastatic clones are related and seeded from the primary tumor; (ii) bone metastases are derived from primary; or (iii) lung metastasis derived from primary. Phylogenetic analysis based on somatic mutations (i.e., substitutions and indels) revealed that, in four of the five mice (P=1.6×¹⁰⁻⁷), the common recent ancestor of the primary tumor and bone metastasis precedes the common recent ancestor of the primary and lung metastasis (bootstrap p-value<10⁻⁷; FIGS. 2A-D), suggesting an earlier metastatic clone that seeds bone metastasis, while lung metastases are derived from a later clone more closely related to the sampled primary tumor.

To investigate the molecular phenotype of bone metastases compared with prostate tumors and metastases to other tissue sites, RNA sequencing analysis was performed on a comprehensive panel of primary tumors (n=19) and macro-dissected metastases from bones (n=12), lungs (n=11), livers (n=5), and lymph nodes (n=4) of 11 independent “high metastatic” NPK-CAG^{YFP} mice (Dataset 1). Principal component analyses (PCA) showed that the bone metastases consistently clustered separately from the primary tumor and lung metastases, as well as metastases to the other tissue sites (FIGS. 2A, 3A and S4A). Analyses of differentially-expressed genes that significantly contribute to principal component 1 further demonstrate a distinct molecular phenotype in bone metastases compared with primary tumors and metastases to other tissues (FIGS. 3B and S4B).

Using these RNA sequencing profiles, a mouse “bone metastasis signature” was defined by comparing the gene expression profiles from the bone metastases (n=12) with those of the primary tumors (n=19) of the NPK-CAG^{YFP} mice (Dataset 1. Since this bone metastasis signature was derived from macro-dissected tumor and metastases that inevitably include non-tumor cells, the cell-intrinsic features between the signature and the single-cell RNA sequencing data was assessed (see FIGS. 1D-F), isolating the YFP-expressing primary tumor (black) and the bone metastatic (dark grey) clusters from the single-cell data for enhanced resolution (FIG. 3C).

The bone metastasis signature (i.e., as defined from the bulk RNA sequencing) was significantly enriched in the single-cell bone metastatic cluster relative to the primary tumor cluster (two-sample two-tailed Welch t-test, p-value=6.1×10⁻⁶, FIG. 3D), indicating that the bone metastatic cells were strongly driving this bone metastasis signature. This was further evident by GSEA in which the bone metastasis signature from the bulk RNA sequencing analyses was used to query the reference signature from the single-cell bone metastasis versus primary tumor samples (GSEA positive leading edge NES 7.83, p-value<0.001 and GSEA negative leading edge NES −3.78, p-value<0.001; FIG. 3E). These findings thus indicate that a “bone metastasis signature” from the NPK-CAG^{YFP} mice is driven significantly, albeit not likely not exclusively, from the bone metastatic cells.

The next inquiry was whether the mouse “bone metastasis signature” is conserved with bone metastases from human prostate cancer. For this, cross species GSEA was performed, comparing the mouse signature with a human signature comprised of primary prostatectomy cases (n=19) and bone biopsies from patients with metastatic prostate cancer (n=19) (Balk signature, Table S2). This revealed that the mouse bone metastasis signature is significantly enriched with the human signature in both the positive (GSEA NES 5.07, p-value<0.001) and negative (GSEA NES −4.44, p-value<0.001) leading edges (FIG. 3F, Dataset 1) and is therefore well-conserved with its human counterpart. Taken together, these molecular investigations demonstrate that bone metastases from the NPK-CAG^{YFP} mice arise from a distinct sub-clone of the primary tumor and have a unique molecular profile compared with metastases to other tissue sites, and identify a cell-intrinsic signature of mouse bone metastasis that is well-conserved with human prostate cancer bone metastases.

### C. MYC Activity is Up-Regulated in Prostate Cancer Metastasis

To identify conserved biological pathways associated with prostate cancer bone metastases, cross species GSEA was performed on pathways comparing the mouse bone metastasis signature with two independent signatures representative of human bone metastasis. In particular, the Balk cohort was used (Table S3); this compares primary prostatectomy and bone biopsies from patients that had been living with metastatic prostate cancer (see above, Table S2), as well as a second human bone metastasis signature, which compares primary tumors (n=14) and bone metastases (n=20) collected from a rapid autopsy cohort and therefore representative of patients that had succumbed to prostate cancer (FHCRC cohort, Table S2). Pathway enrichment analysis was performed using the Hallmarks and C2 (i.e., KEGG, Reactome and BioCarta) databases on all three bone metastasis signatures (i.e., the mouse signature from the NPK-CAG^{YFP} model and the two human signatures from the Balk and FHCRC cohorts) and subsequently utilized these pathway signatures for cross species pathway-based GSEA comparing pathways from the mouse bone metastasis signature with those from either the Balk or FRCRC human bone metastasis signatures (FIGS. 4A, S5A, Dataset 3). This analysis revealed significant similarity of activated pathways in the mouse bone metastasis signature with the human bone metastasis signatures from Balk (GSEA NES 3.77, p-value<0.001; FIG. 4A) as well as from FHCRC (GSEA NES 3.2, p-value<0.001; FIG. S5A). Biological pathways that significantly contributed to these similarities (i.e., belong to the leading edges from GSEA comparisons) were further integrated using the Stouffer method, which identified those that were both significantly enriched and conserved between all mouse and human bone metastasis signatures (FIG. S5B, Dataset 3).

Among the relatively few biological pathways that fit these stringent criteria (n=31 pathways), the most significantly enriched across all three signatures was the MYC pathway (FIG. S5B, Dataset 3). Notably, MYC was also among the most significantly enriched pathways comparing primary tumors from NPK-CAG^{YFP} mice with or without bone metastases (see FIG. S3B).

It was confirmed by the methods herein that the Hallmarks MYC pathway is significantly enriched in all three signatures, namely the NPK-CAG^{YFP} mouse bone metastasis (GSEA NES 5.24, p-value<0.001), Balk human bone metastasis (GSEA NES 3.68, p-value<0.001), and FHCRC human bone metastasis (GSEA NES 5.84, p-value<0.001) signatures (FIG. S5C). In addition, the results showed that MYC was also up-regulated in the mouse and human bone metastasis signatures using two additional signatures; namely, a widely used signature of canonical MYC target genes (called the Dang MYC pathway, NPK-CAG^{YFP} GSEA NES 3.99, Balk GSEA NES 3.4, and FHCRC GSEA NES 3.21, all p-values<0.001), and a signature of oncogenic MYC targets (called the Sabo Myc pathway, NPK-CAG^{YFP} GSEA NES 4.56, p-value<0.001, Balk GSEA NES 4.92 p-value<0.001, and FHCRC GSEA NES 2.75, p-value=0.0028) (FIG. S5D-E).

To compare MYC activity levels in human prostate cancer metastases to primary tumors (where MYC activity is defined based on the GSEA enrichment of the MYC pathway from the Hallmarks database, as in FIGS. 4A, S5B-C), single-sample GSEA was first performed using the MYC Hallmark pathway genes to query each of the metastases from mCRPC in the Stand Up to Cancer cohort (SU2C (44); n=270, Table S2) and each of the primary tumors from The Cancer Genome Atlas prostate adenocarcinoma cohort (TCGA; n=497 Table S2) (FIG. S5F). Distributions of MYC activity (i.e., single-sample GSEA enrichment) levels were then compared between these cohorts as depicted by a violin plot. This revealed significant up-regulation of MYC activity in human prostate cancer metastases compared with primary tumors (two-sample one-tailed Welch t-test, p-value<10′; FIG. 4B). Notably, the SU2C cohort includes the bone metastases (n=74) as well as metastasis to other tissue sites (n=196) and MYC activity was up-regulated across all metastases not only in the bone metastases (FIG. S5F). Furthermore, immunohistochemical analysis of patient samples from mCRPC revealed that MYC protein, which is known to be up-regulated in prostate cancer, was robustly expressed in human prostate cancer bone metastases (n=12), as well as metastases to other tissue sites (n=22; FIGS. 4C, S5G; Table S3). Taken together, these findings show that MYC activity and protein levels are up-regulated in prostate cancer metastasis including in bone metastases.

### D. MYC is Necessary for Prostate Cancer Bone Metastasis

MYC is also expressed at high levels in human prostate cancer cells, including the PC3 line, which was derived from a bone metastasis and is known to grow in bone when implanted orthotopically in xenograft models. Therefore, an investigation was made into whether MYC expression is necessary for tumor growth in bone by silencing its expression in PC3 cells using two independent MYC shRNAs or a control shRNA (FIGS. 4D-F, S6A-B).

First, the PC3 cells were engineered to express both luciferase and green fluorescent protein (GFP) (herein called PC3-Luc-GFP cells), such that their growth could be monitored in vivo using IVIS imaging and ex vivo using GFP fluorescence (FIG. 4G-I, S6C-E, and see Methods). Following lentiviral transduction with the MYC shRNAs (shMYC#1 or shMYC#2) or the control shRNA (shControl), the PC3-Luc-GFP cells were implanted into the tibia of NOD-SCID mouse hosts, to monitor bone growth (n=10/group, FIGS. 4G-I), or subcutaneously into the flank, to monitor tumor growth (n=5/group, FIGS. S6C-E).

As evident by in vivo IVIS imaging, silencing of MYC inhibited tumor growth specifically in bone and not in the flank (two-way ANOVA with Sidak's multiple comparisons against shControl p-value<0.0001; FIGS. 4G and S6C). Furthermore, the tibias of mice injected with the shControl expressing PC3-Luc-GFP cells compared with the corresponding shMYC#1- or shMYC#2 expressing cells exhibited more robust ex vivo fluorescence, while histological analysis revealed large YFP-expressing tumors specifically in the shControl—expressing PC3-Luc-GFP tumors (FIG. 4I).

To investigate directly whether MYC is necessary for metastasis to bone, and not only for tumor growth in bone, an in vivo allograft model of bone metastasis was established. In particular, a cell line from a femur metastasis of an NPK-CAG^{YFP} mouse was isolated and selected for enhanced potential to metastasize to bone when implanted into Nude male hosts in vivo (hereafter called NPK bone cells, see Methods, FIG. S7). Unlike cells from the non-metastatic NP tumors, intracardiac injection of the NPK bone cells into host mice leads to robust metastases to bone, as well as to lung and other soft tissues, which are readily detected by ex vivo fluorescence imaging (FIG. S7).

To ask whether MYC is necessary for metastasis in vivo in this model, lentiviral transduction was used to introduce two independent Myc shRNAs (shMyc#1 or shMyc#2) or a control shRNA (shControl) into the NPK bone cells and then monitored the occurrence of metastasis following intracardiac injection (FIGS. 5A-E, S8A-B). Whereas the shControl NPK bone cells developed extensive metastases to bone, as well as to lung and other soft tissues, the shMyc#1 or shMyc#2-expressing NPK bone cells showed a significant reduction in the number of metastases to bone but no significant reduction in metastases to lung (n=8, one-way ANOVA with Dunnett's multiple comparisons against shControl p-value<0.05, FIGS. 5C-D). The reduced incidence of bone metastases in the shMyc#1 or shMyc#2-expressing NPK bone cells was evident in all of the bones examined, namely spine, pelvis, femur, tibia and humerus (FIG. 5C). Furthermore, ex vivo fluorescence and histological analyses of bones from mice injected with the shControl NPK bone cells revealed large tumors expressing high levels of Myc as well as the YFP reporter, whereas the bones from mice implanted with shMyc#1 or shMyc#2 cells had smaller or no tumors, with coincidently lower expression of Myc and the YFP reporter (FIG. 5E). Taken together, these findings support the conclusion that MYC is necessary for prostate cancer metastasis in vivo, and particularly for metastasis to bone.

### E. A Gene Signature Prognostic for Time to Metastasis in Primary Prostate Tumors

The present findings demonstrating that MYC is enriched in and necessary for prostate cancer metastasis, together with the present observation that Myc activity is up-regulated in NPK-CAG^{YFP} primary tumors that have increased propensity to metastasize (see FIG. S3B), prompted the identification of a MYC-related gene signature associated with metastasis-progression in human prostate cancer. The overall strategy herein was to select for genes that were: (i) correlated with MYC expression in human prostate cancer metastases; (ii) up-regulated in both mouse and human bone metastasis signatures; and (iii) associated with adverse outcome for metastasis (FIG. 6A). For the first step, genes correlated with MYC expression were identified using the PROMOTE human cohort (for PROstate Cancer Medically Optimized Genome-Enhanced ThErapy), which is comprised of tissue biopsies of prostate cancer metastases from patients with mCRPC (n=77), many of which are bone metastasis (n=55) (Table S2). First, genome-wide correlation was performed in the PROMOTE dataset to identify genes that are both significantly and positively correlated with MYC mRNA expression (n=559 genes, “PROMOTE-559”; Spearman rho>0.5, FDR p-value<0.0001, FIG. 6B). Next, GSEA was performed to query the mouse NPK-CAG^{YFP} and human Balk bone metastasis signatures with the PROMOTE-559 genes (FIGS. 59A-B). These analyses revealed significant enrichment of PROMOTE-559 in the mouse bone metastasis signature (GSEA NES 4.65, p-value<0.001, leading edge genes shown in y-axis of FIG. 6B and FIG. S9A), as well as the human bone metastasis signature (GSEA NES 4.35, p-value<0.001, leading edge genes shown in z-axis of FIG. 6B and FIG. S9B).

Integration of these leading edge genes identified 55 that were: (i) positively correlated with MYC expression in metastases, and (ii) overexpressed in both the mouse and human bone metastasis signatures (i.e., belonging to the leading edges from the mouse and human GSEA analyses) (FIG. 6B); we termed this gene signature “META-55.” To narrow further the META-55 genes to those most likely to be associated with adverse outcome for metastasis, these genes were ranked based on univariable Cox proportional hazards modeling in the TCGA cohort using metastasis-free survival as end point. This analysis identified 16 genes whose expression was most significantly associated with metastasis-free survival (Wald p-value=1×10′, FIG. S9C); this gene signature was termed, “META-16.”

It is noted that in the various analyses performed to validate the META-55 and META-16 gene signatures, while both were significant, META-16 (which is a subset of META-55) consistently outperformed even the META-55 (see FIGS. 6, S9D, S10, and S12).

Enrichment of the META-16 (and META-55) gene signatures and their correlation with MYC activity in the mouse bone metastatic cells was evident from analyses of the single-cell RNA sequencing data comparing the YFP-expressing primary tumor and bone metastatic clusters (FIGS. 6C, 6D, S10A-B, and see FIG. 3C). Notably, the primary tumor cluster was itself heterogenous and comprised of two major sub-clusters, and projecting the bone metastasis cluster onto the primary tumor cluster revealed similar heterogeneity (FIG. S10A and see FIG. 1E). As visualized by UMAP, MYC activity was up-regulated in a subset of cells in the primary tumor cluster and a corresponding subset of cells in the bone metastasis cluster (FIG. 6C). Most notably, this expression was precisely correlated with up-regulation of both META-16 and META-55 in these clusters (Spearman correlation p-value<2.2×10⁻¹⁶; FIGS. 6C, 6D, S10B).

To evaluate the expression of META-16 and META-55 in primary tumors versus metastasis, a single-sample GSEA was first performed to evaluate enrichment of META-16 and META-55 in each of the primary tumors from the TCGA cohort (n=497) and each of the metastases from the SU2C cohort (n=270) (FIG. S10C, Table S2). Comparison of distributions of enrichment levels of META-16 and META-55 between these cohorts as depicted by a violin plot revealed that they were highly significantly up-regulated in the prostate cancer metastases from the SU2C cohort compared with primary tumors from TCGA cohort (for META-16, two-sample one-tailed Welch t-test p-value<10⁻¹²⁵ and for META-55, two-sample one-tailed Welch t-test p-value<10′; FIG. 6D, S10D). To demonstrate the non-random ability of META-16 to distinguish primary tumors in the TCGA cohort from the CRPC metastatic samples in the SU2C cohort by selecting a random (equally sized, n=16) group of genes and compared their estimated activity levels between TCGA and SU2C cohorts (two-sample one-tailed Welch t-test p-value=0.003, FIG. S9D).

Enrichment in metastases was further evident from the striking up-regulation of individual genes included in the META-16 and META-55 signatures across patient samples in the metastases from SU2C when compared to primary tumors from the TCGA (FIGS. 6F, S10E). Similar to the findings for MYC activity (see FIG. 4B), the META-16 and META-55 while enriched in the bone metastases (n=74) were also up-regulated in metastasis to other tissue sites (n=196) in the SU2C cohort (FIG. 6E, F, S10C-E). Furthermore, META-16 and META-55 also showed robust up-regulation in a subset of primary tumors at each Gleason score (FIG. 6F, S10E) and are not specific to bone metastases (FIG. S10F). Therefore, results showed that the META-16 and META-5 signatures are enriched in but not exclusive to prostate cancer metastases.

The mRNA expression levels of the individual META-16 genes were further validated by real-time quantitative PCR using RNA prepared from frozen specimens of 5 bone metastases and 10 primary prostate tumors (all Gleason 9, the CUIMC mRNA cohort), which revealed increased expression of several of the META-16 genes in bone metastases relative to primary tumors (two-tailed Mann-Whitney test, p-value<0.05, FIG. S11A). Additionally, one of the META-16 genes was selected for validation at the protein level, namely, ATAD2 (for ATPase Family AAA Domain Containing 2), which is a bromodomain protein that is co-amplified with MYC and a cofactor of MYC as well as androgen receptor. Immunohistochemical analyses of ATAD2 using the Bern/CUIMC IHC cohort which includes brain metastases (n=6) and matched primary tumors, and bone metastases (n=4) revealed robust expression of ATAD2 particularly in the metastases (FIG. S11B). Lastly, results showed that silencing MYC in metastatic cell lines from human (shControl versus shMYC-PC3-Luc-GFP) and mouse (shControl versus shMyc NPK bone) prostate cancer resulted in lower levels of the META-16 genes (FIG. S11C-D), supporting the correlation of these genes with MYC expression.

The findings showing that the META-16 (and META-55) signatures are: (i) up-regulated in prostate cancer metastasis including but not exclusively in bone metastasis (e.g., FIG. 6E); (ii) expressed in a subset of primary tumors at all Gleason grades (e.g., FIG. 6F); and (iii) enriched in specific subsets of primary tumor cells (e.g., FIG. 6D) prompted the inquiry into whether expression of META-16 (and META-55) is associated with risk of metastases in primary prostate tumors.

To determine the answer, two independent retrospective cohorts of primary prostatectomy cases with clinical outcome data were used (the Mayo cohort and the JHMI cohort, Table S2) (the Mayo cohort: Karnes R J, Bergstralh E J, Davicioni E, Ghadessi M, Buerki C, Mitra A P, et al. Validation of a genomic classifier that predicts metastasis following radical prostatectomy in an at risk patient population. J Urol 2013; 190(6):2047-53 doi 10.1016/j.juro.2013.06.017); (the JHMI cohort: Ross A E, Johnson M H, Yousefi K, Davicioni E, Netto G J, Marchionni L, et al. Tissue-based Genomics Augments Post-prostatectomy Risk Stratification in a Natural History Cohort of Intermediate- and High-Risk Men. Eur Urol 2016; 69(1):157-65 doi 10.1016/j.eururo.2015.05.042).

Patients in the Mayo cohort (n=235) had undergone radical prostatectomy between 2000 and 2006 and had a median follow up of 7 years with 76 patients developing metastasis. Similarly, patients in the JHMI cohort (n=260) had undergone radical prostatectomy between 1992 and 2010 and had a median follow up of 9 years with 99 patients developing metastasis.

The association of META-16 (and META-55) was tested with metastasis free survival, which is now a clinical endpoint for prostate cancer clinical trials. Hierarchical consensus clustering was performed on META-16 (and META-55) expression levels, which grouped the patients into two clusters corresponding to low or high levels of the META-16 (and META-55) signatures (FIGS. 6G, 6H and S12A-B). Kaplan-Meier survival analyses was then performed to assess the differences between these patient clusters having low or high levels of META-16 (and META-55) with respect to metastasis-free survival. This analysis demonstrated that patients with high expression levels of META-16 (and META-55) have a shorter time to metastasis than those with low expression levels (META-16 Mayo log-rank p-value<0.0001 and JHMI log-rank p-value<0.0001; META-55 Mayo log-rank p-value=0.00018 and JHMI log-rank p-value=0.00056, FIG. 6I, 6J and Fig. S12C-D).

Interestingly, multivariable Cox proportional hazards model to adjust for age, pathological Gleason score/grade at diagnosis, pre-PSA, seminal vesicle invasion SVI, lymph node invasion LNI, and extra-prostatic extension EPE) of the META-16 (and META-55) gene signatures showed that these signatures were independently associated with metastasis-free survival (META-16 metastasis-free survival MAYO hazard ratio HR=3.11, 95% confidence interval CI: 1.71-5.63, p-value=0.0001 and JHMI HR=2.16, 95% CI: 1.38-3.38, p-value=0.0006; META-55 metastasis-free survival MAYO HR=1.8, 95% CI: 1.05-3.07, p-value=0.03 and JHMI HR=1.8, 95% CI: 1.18-2.74, p-value=0.006) but not with prostate-cancer specific mortality (META-16 prostate-cancer specific mortality MAYO HR=2.26, 95% CI: 0.98-5.23, p-value=0.05 and JHMI HR=1.63, 95% CI: 0.82-3.23, p-value=0.15; META-55 prostate-cancer specific mortality MAYO HR=1.61, 95% CI: 0.72-3.6, p-value=0.24 and JI-IMI HR=1.55, 95% CI: 0.8-3.00, p-value=0.18 (FIG. 6K, S12E). These findings suggest that META-16 (which is a subset of META-55) and the META-55 signatures may be used in prognostic tests for identifying patients with localized disease that have a higher risk of prostate cancer metastasis.

### F. Discussion

The current study describes the generation of a genetically-engineered mouse model (GEMM) that develops bone metastases with >40% efficiency, and was shown to overcome the earlier lack of high-efficiency models to develop bone metastasis in the context of the native tumor microenvironment and during the natural evolution of tumor progression in vivo. Indeed, the high penetrance of bone metastasis in NPK-CAG^{YFP} mice (for Nkx3.1^{CreERT2/+}; Pten^{flox/flox}; Kras^{LSL-G12D/+}; R26R-CAG-^{LSL-EYFP/+}) permits investigation of the biological and molecular processes associated with bone metastasis as they arise de novo during tumor progression in vivo, as well as their relationship to primary tumors and metastases to other tissue sites.

Furthermore, conservation of the molecular features of mouse bone metastasis in NPK-CAG^{YFP} mice with human prostate cancer metastasis has permitted investigation of molecular programs that are relevant for human prostate cancer metastasis, and in particular has led to the identification of gene signatured, META-16 and META-55, that have prognostic significance for identifying patients with a higher risk of prostate cancer metastasis. Thus, in certain embodiments, these unique and novel GEMMs of bone metastasis have substantial impacts on clinicians' and researchers' understanding of the biological, molecular, and clinical features of bone metastasis.

In certain embodiments, the NPK-CAG^{YFP} mouse models are different than those currently known, and uniquely useful in the management of cancer therapies for several reasons that contribute to their unique phenotype.

First, gene recombination is mediated by an Nkx3.1-driven inducible Cre, which both models heterozygous loss of Nkx3.1 and achieves tumor induction precisely in adult prostatic luminal cells, as are relevant for human cancers, and in particular for prostate cancer. Notably, both primary tumors and metastases from the NPK-CAG^{YFP} mice display a luminal phenotype.

Second, lethal prostate cancer in the NPK-CAG^{YFP} mice arises as a consequence of conditional loss of function of Pten, to model PI3 kinase activation, and conditional activation of mutant Kras, to model activation of RAS signaling, which are known to be frequently co-activated in lethal prostate cancer in humans. Notably, while KRAS mutations are rare in prostate cancer, activation of RAS signaling is common particularly in aggressive tumors and metastasis. The results herein show that activation of Kras in the NPK-CAG^{YFP} mice is an effective surrogate for modeling activation RAS signaling in human prostate cancer.

Third, the NPK-CAG^{YFP} mice were shown to incorporate a robust fluorescent reporter, R26R-CAG-^{LSL-EYFP/+}, that allows for ready visualization of bone metastases that would otherwise have been exceedingly difficult to detect in the whole organism.

In combination, these features contribute to the highly aggressive prostate cancer phenotype of the NPK-CAG^{YFP} mice, including their highly penetrant and readily detectable bone metastases.

In certain embodiments, the current model complements previous GEMM models that have been informative for understanding bone metastases, despite their relatively low penetrance (and relative disadvantage when compared to the present embodiments). For example, a GEMM based on overexpression of Hepsin in the context of the SV40-based Lady mice was reported to develop bone metastases in approximately 10% of cases (Klezovitch et al., Cancer Cell 2004; 6(2):185-95 doi 10.1016/j.ccr.2004.07.008). Analysis of this model has shown that Hepsin is targetable using a small molecule inhibitor.

Another GEMM, based on telomere dysfunction in the context of combined loss-of-function of Smad4 and Pten, has been reported to develop bone metastasis coincident with extensive DNA damage (Ding et al., Cell 2012; 148(5):896-907 doi 10.1016/j.cell.2012.01.039); this is notable since a major pathway activated the bone metastases of the NPK-CAG^{YFP} mice is DNA repair.

Furthermore, previous analyses of other GEMMs of metastatic prostate cancer, together with the present study, highlight the important role of MYC for prostate cancer metastasis and suggest that while MYC may be necessary for prostate cancer metastasis, it is unlikely sufficient for metastases specifically to bone. Two other GEMMs based on activation of MYC have been shown to develop bone metastasis with rare penetrance (Hubbard et al., Cancer Res 2016; 76(2):283-92 and Magnon et al., Science 2013; 341(6142):1236361 doi 10.1126/science.1236361; while the “rapid-CAP model,” based on combined loss of Pten and p53, promotes prostate cancer metastasis via Myc although this study did not report the occurrence of bone metastases (Cho et al., Cancer Discov 2014; 4(3):318-33 doi 10.1158/2159-8290.CD-13-0346 and Nowak et al., Cancer Discov 2015; 5(6):636-51 doi 10.1158/2159-8290.CD-14-1113. It seems likely that the highly metastatic phenotype and particularly the high penetrance of bone metastasis in the NPK-CAG^{YFP} mice reflects the convergence of RAS signaling for MYC activation. Notably, while MYC is up-regulated at all stages of prostate cancer including early disease, both activation of RAS signaling and MYC amplification are more prevalent in prostate cancer metastases relative to primary tumors. Indeed, among the broad network of MYC activities, collaboration of RAS signaling and MYC has been observed in other cancer types.

It is noted in this study that although bone metastases in NPK-CAG^{YFP} mice occur in “high-metastatic” contexts they arise from an earlier sub-clone of the primary tumor than metastases to other sites, suggesting that bone metastases may be seeded earlier but may take longer to be overtly detected. This parallels the scenario in the human clinical setting since although prostate cancer bone metastasis is much more prevalent than metastases to visceral tissues, the latte are associated with worse clinical outcome.

In certain embodiments, the clinical significance of the present analyses of bone metastasis in the NPK-CAG^{YFP} mice is highlighted by the identification of the gene signatures herein, e.g., META-16 and META-55, that are prognostic for metastases. In particular, the present cross species analysis of NPK-CAG^{YFP} mice with human prostate cancer metastases has identified the MYC-correlated signatures herein, which are significantly elevated in metastasis, and particularly associated with adverse outcome for prostate cancer metastasis.

In certain embodiments, the signatures discussed herein are complementary to other prognostic signatures of prostate cancer, such as Decipher GX (which is also associated with risk of metastasis), and the Prolaris CCP score (which is associated with prostate cancer specific survival. In various embodiments, the META-16 and META-55 signatures, potentially in conjunction with these other signatures, can be used to identify patients with prostate cancer that are destined to develop metastasis, and thereby have significant clinical utility.

### G. Methods

### i) Generation and Phenotypic Analyses of a Genetically Engineered Mouse Model of Bone Metastasis

All experiments using animals were performed according to protocols approved by the Institutional Animal Care and Use Committee (IACUC) at Columbia University Irving Medical Center. For these studies, Nkx3.1^{CreERT2/+}; Pten^{flox/flox}; Kras^{lsl-G12D/+} (NPK) mice (25) were crossed with the Rosa-CAG-LSI-EYFP-WPRE reporter allele (23) to obtain the experimental Nkx3.1^{CreERT2/+}; Pten^{flox/flox}; Kras^{lsl-G12D/+} R26R-CAG-^{LSL-EYFP/+} (NPK-CAG^{YFP}) mice and the control (non-metastatic) Nkx3.1^{CreERT2/+}; Pten^{flox/flox}; Kras^{+/+}; R26R-CAG-^{LSL-EYFP/+} (NP-CAG^{YFP}) mice used herein. The NPK mice have been maintained in our laboratory on a predominantly C57BL/6 background; the Rosa-CAG-LSL-EYFP-WPRE mice were obtained from Jackson Laboratories on a C57BL/6 background (Stock No: 007903).

All studies were done using littermates that were genotyped prior to tumor induction; since the focus of the study was prostate cancer, only male mice were used. Mice were induced to form tumors at 2-3 months of age by administration of tamoxifen (Sigma-Aldrich, Allentown, Pa., USA) using 100 mg/kg once daily for 4 consecutive days. Control (non-tumor induced) NPK-CAG^{YFP} mice were delivered corn oil (vehicle for tamoxifen) and otherwise monitored in parallel with tumor-induced mice.

Following tamoxifen-induction, mice were monitored daily and euthanized when their body condition score (65) was <1.5, or when they experienced body weight loss 20% or signs of distress, such as difficulty breathing or bladder obstruction. Where indicated, surgical castration was performed 1 month after tumor induction.

At the time of sacrifice, YFP-positive prostatic tumors and metastases from non-prostatic tissues were visualized and quantified by ex vivo fluorescence using an Olympus SZX16 microscope (Ex490-500/Em510-560 filter). For accurate visualization of the fluorescent signal, a composite image was made by superimposing a bright field image (20% transparent) on the fluorescent image of the same area. For detection of bone metastases, muscle and connective tissue surrounding the bones of the vertebrae, pelvis, femurs, tibiae, humeri, ulnae, radii and calvariae were removed prior to ex vivo fluorescence examination. Ribs were not evaluated given extensive metastasis in surrounding soft tissues that might confound detection of bone metastases in ribs.

For histological and immunohistochemical analyses, dissected tissues were fixed in 10% formalin (Fisher Scientific, Fair Lawn, N.J., USA). Bones were then decalcified for three weeks in 15% EDTA pH7.0 solution (Sigma E5134), and changed daily. For isolation of genomic DNA or RNA, metastases were visualized by ex vivo fluorescence of YFP and macrodissected prior to analyses. For bulk RNA analyses or preparation of genomic DNA the samples were snap frozen in liquid nitrogen; for single-cell RNA sequencing the samples were processed directly (as per below).

Histopathological and immunohistochemical analyses were done using 3 μm paraffin sections as described. Histopathological examinations of hematoxylin and eosin (H&E)-stained sections from mouse prostate tumors and metastases were performed blinded by two independent pathologists (AMD and MAR). For immunostaining, 3 μm paraffin sections were deparaffinized and rehydrated, followed by antigen retrieval for most antibodies in citrate-based antigen unmasking solution (Vector Labs, Burlingame, Calif., USA) or in Tris-EDTA pH8.0 for the Myc antibody. Slides were blocked in 10% normal goat serum, then incubated with primary antibody overnight at 4° C., followed by incubation with secondary antibody for 1 hour.

For immunohistochemistry, the signal was enhanced using the Vectastain ABC system and visualized with NovaRed Substrate Kit (both from Vector Labs). Slides were counterstained with hematoxylin and mounted with Permount (Fisher Scientific), and images were captured using an Olympus VS120 whole-slide scanning microscope. For immunofluorescence staining, sections were counterstained with DAPI solution (BD Biosciences, Franklin Lakes, N.J., USA) and mounted with Vectashield mounting medium for fluorescence (Vector Labs). Images were captured using a Leica TCS SP5 confocal microscope. All antibodies used in this study, as well as antibody dilutions, are described in Table S4.

Analysis of RNA expression was done by quantitative real time PCR using the QuantiTect SYBR Green PCR kit (Qiagen, Germantown, Md.). Sequences of all primers used in this study are provided in Table S5. Western blotting was performed using total protein extracts; antibodies and dilutions are provided in Table S4.

### ii) RNA Sequencing Analyses of Mouse Tumors and Metastases

Transcriptomic analysis of bulk tissues was done on primary tumors (n=19) and matched macrodissected metastases from lung (n=11), liver (n=5), lymph nodes (n=4), or bone (n=12) from 11 independent NPK-CAG^{YFP} mice. RNA was prepared from snap-frozen tissues the using MagMAX-96 total RNA isolation kit (ThermoFisher, Bridgewater, N.J., USA). Total RNA was enriched for mRNA using poly-A pull-down; only RNA samples having between >200 ng and 1 μg and with an RNA integrity number (R1N) >8 were used. Libraries were made using an Illumina TruSeq RNA prep-kit v2 and sequenced using an Illumina HiSeq2500 by multiplexing samples in each lane, which yields targeted number of single-end/100 bp reads for each sample, as a fraction of 180 million reads for the whole lane. Reads were aligned to the mm9 mouse genome using Kallisto, and RNA-seq raw counts were normalized and the variance was stabilized using DESeq2 package (Bioconductor) in R-studio 0.99.902, R v3.3.0 (The R Foundation for Statistical Computing, ISBN 3-900051-07-0).

Differential gene expression signatures were defined as a list of genes ranked by their differential expression between any two phenotypes of interest (e.g., metastasis vs primary tumor) estimated using a two-sample two-tailed Welch t-test. A complete list of differentially expressed genes is provided in Supplementary Dataset 1. For comparison with human genes, mouse genes were mapped to their corresponding human orthologs based on the homoloGene database (NCBI) so that mouse-human comparisons were done using the “humanized” mouse signatures. For gene set enrichment analysis (GSEA) (36), normalized enrichment score (NES) and p-value were estimated using 1,000 gene permutations. Pathway enrichment on the differential signatures of interest was performed using GSEA to query the Molecular Signatures Database (MSigDB), available from the Broad Institute, including the C2 (KEGG, Reactome, and BioCarta) and Hallmark pathway datasets. A complete list of differentially expressed pathways is provided in Supplementary Dataset 3.

iii) Whole Exome Sequencing Analysis of Mouse Tumors and Bone Metastases

Whole exome sequencing (WES) was done on matched trios of primary tumors, lung metastases, and bone metastases, as well as tails (as a control) from five independent NPK-CAG^{YFP} mice. Genomic DNA was isolated from snap-frozen tissues using the DNeasy Blood & Tissue Kit (Qiagen) and DNA quality confirmed by gel electrophoresis and visual observation of a clear, non-degraded main band of DNA. Whole exome sequencing was performed by BGI Americas Corporation using the HiSeq4000 platform and Agilent Sure Select Mouse Exon kit (50 Mb) for exome capture to produce paired-end sequenced data of up to 150 bp read length. The resulting average sequencing depth was more than 80×, and reads were mapped to the mouse mm10 genome build using bwa. Substitutions and indels were called using SAVI; only variants with a mutant allele frequency of 5% or greater were included for further analysis. A complete list of single nucleotide variants is provided in Supplementary Dataset 4.

Evolutionary trees were reconstructed using somatic mutations (i.e., substitutions and indels). In particular, the number of somatic mutations specific to or shared between primary tumors, lung metastases, and bone metastases were used to build evolutionary trees so that the lengths of the branches indicate the number of specific or shared (branches and trunks in FIG. 2) somatic mutations in each sample.

The significance of the phylogeny of the evolutionary tree was tested using bootstrap test. For this, within one trio (primary, lung, bone metastases), given the observed somatic mutation matrix, the mutations were randomly shuffled. An evolutionary tree was then reconstructed using this new somatic mutation matrix and the topology of this tree was compared to that of the original tree. If there are m mutations shared between primary tumor and lung met, and n mutations between primary and bone met, then the tree in which m-n mutations is less than in the original tree is given a score of 0; all others are given the value 1. This procedure of resampling and the subsequent tree reconstruction was repeated 1,000 times, and the percentage of times one tree is given a value of 1 is noted as bootstrap-derived p-value. Representative combined phylogeny was then constructed reflecting consistent evolutionary patterns across all trees and its meta-analysis p-value was calculated using Fisher's method through combining bootstrap-derived p-values from individual trees.

### iv) Single-Cell RNA Sequencing Analyses of Mouse Tumors and Bone Metastases

Single-cell RNA sequencing was done on freshly dissected prostate tumor and two pooled macrodissected bone metastases from an NPK-CAG^{YFP} mouse, where single cells were captured and barcoded using the 10× Genomics Chromium platform, and libraries were sequenced on an Illumina NovaSeq. First, the tissues were minced with scissors and enzymatically digested for 15 minutes at 37° C. in a mixture containing 1× collagenase/hyaluronidase, 0.5 U/mL dispase II and 0.1 mg/mL DNAse 1 in DMEM-F12 media, followed by addition of 0.025% trypsin/EDTA for another 15 minutes (all obtained from Stem Cell Technologies, Cambridge, Mass., USA). Cells were resuspended in cold 10% FBS DMEM-F12, filtered through a 40 μM cell strainer and collected by centrifugation at 350×G in an Eppendorf 5810R tabletop centrifuge for 5 min at 4° C. After a 5-minute incubation in cold 1× Red Blood Cell Lysis Buffer (ThermoFisher, Bridgewater, N.J., USA) cells were diluted 4-fold in cold PBS, centrifuged as before and resuspended in DMEM-10% FBS for cell counting and viability analysis.

Cells were counted using a Countess II Automated Cell Counter (ThermoFisher) and 10,000 cells with over 70% viability were loaded into a 10× Genomics Chromium Controller for capture and barcoding following the 10× Genomics Single Cell Protocol, as described by the manufacturer (10× Genomics, Pleasanton, Calif., USA), with subsequent RNA sequencing using Illumina NovaSeq. Reads were mapped to the mouse mm9 genome and processed with the CellRanger pipeline.

Single-cell RNA-seq raw counts were normalized and the variance was stabilized using DESeq2 package (Bioconductor) in R-studio 0.99.902, R v3.3.0 (The R Foundation for Statistical Computing, ISBN 3-900051-07-0). The uniform manifold approximation and projection (UMAP)(35) dimensionality reduction technique implemented in Python was used to cluster primary and metastatic single-cell RNA sequencing data. UMAP visualizations were constructed as described (74); the code for visualization is available at https://github.com/simslab.

### v) Description of Human Patient Cohorts

All studies using human tissue specimens were performed according to protocols approved by the Human Research Protection Office and Institutional Review Board (IRB) at the respective institutions. Published human patient cohorts used for discovery (i.e., the Balk, FHCRC, and PROMOTE cohorts) or validation (i.e., the TCGA, SU2C, JMH1, and MAYO cohorts) are described in Table S2. An additional cohort was used from FHCRC of bone or soft tissue metastases obtained at autopsy from patients that had died from metastatic castration-resistant prostate cancer (n=138; 98 of the cases are in GEO: GSE126078). These human cohorts include two independent retrospective case-cohorts for validation of clinical outcome, which were retrieved from the Decipher GRID registry (MAYO cohort: GSE62116 (54) and the JHMI cohort: GSE79957). Patients in the MAYO cohort (n=235) had undergone radical prostatectomy between 2000 and 2006 and were identified from the Mayo Clinic tumor registry for a case-cohort study design; median follow up was 7 years with 73 patients developing metastasis. The JHMI cohort is a retrospective case-cohort design of 260 men who had undergone radical prostatectomy between 1992 and 2010 at intermediate or high risk and received no additional treatment until the time of metastasis; median follow up in the cohort was 9 years with 99 patients developing metastasis. Both cohorts were profiled on a Human Exon 1.0 ST Array and hybridization was done in a Clinical Laboratory Improvement Amendments (CLIA/CAP)-certified laboratory facility (GenomeDx Biosciences, San Diego, Calif., USA).

Studies using anonymized human tissue specimens were performed according to protocols approved by the Human Research Protection Office and Institutional Review Board (IRB) at the respective institutions (e.g., Columbia University Irving Medical Center (CUIMC), University of Bern (BERN), or Johns Hopkins Hospital (JHH)). These cohorts were used for quantification of mRNA expression levels (CUIMC cohort) or for immunohistochemical detection of protein expression levels (the JHH cohort and BERN/CUIMC cohorts). The CUIMC RNA expression cohort was comprised of frozen tissues from 5 bone metastatic resections and 10 primary prostate cancer tumors (Gleason score 9) from surgical resections of patients with advanced prostate cancer that had been banked in the Molecular Pathology Shared Resource of the Herbert Irving Comprehensive Cancer Center. RNA was extracted using miRNeasy mini kit (Qiagen) and quantitative real-time PCR was done using the QuantiTect SYBR Green PCR kit (Qiagen, Germantown, Md., USA).

The JHH IHC cohort was comprised of 34 metastatic samples including 12 bone metastatic biopsies of patients diagnosed with advanced prostate cancer. The clinical features of the patients are summarized in Table S3. Immunohistochemistry was done using a Ventana DISCOVERY ULTRA Autostainer and a rabbit monoclonal MYC (Abcam, Cambridge, Mass., USA) and the DISCOVERY anti-HQ HRP kit antibody (Roche, Tucson Ariz.). Immunostaining was quantified using an H-score system obtained by multiplying the intensity of the stain (0: no staining; 1: weak staining; 2: moderate staining; 3: intense staining) by the percentage (0-100) of the cell showing that staining intensity (H-score range 0-300, with 0-100 considered as Low, 101-200 intermediate and 201-300 as High).

The BERN IHC cohort was a retrospective cohort of 6 patients with brain metastatic prostate carcinoma diagnosed between 1991 and 2014 in the Department of Pathology, University Hospital of Bern, Switzerland, and included 4 bone metastases from CUIMC (i.e., BERN/CUIMC IHC cohort). Immunohistochemistry was performed on freshly-cut FFPE-sections from primary tumors and metastatic samples and was assessed as the percentage of viable tumor cells with any nuclear staining above the background. Positive expression was calculated in 5% steps from 5% to 100%. If positive cells were estimated to be less than 5%, expression was considered as 1%. Entirely negative cases were documented as 0%.

### vi) Establishment of a Mouse Allograft Model of Bone Metastasis

To establish a mouse allograft cell line that preferentially metastasizes to bone when grown in vivo in recipient hosts (FIG. S7), cell lines from a femoral bone metastasis were generated from an NPK-CAG^{YFP} mouse by adapting a protocol previously described to isolate cell lines from NPK primary tumors. Briefly, bone metastases in the NPK-CAG^{YFP} mice were visualized by ex vivo fluorescence and then harvested and digested using the method described above for the single-cell RNA sequencing analyses. The cells were cultured for 5 passages using RPMI with 10% FBS as culture media. Once the cells were established in culture, a cell line with enhanced metastasis to bone was generated by passaging in nude mouse hosts. Specifically, the original cells were introduced via intracardiac injection into NCr nude mice (male, Taconic, Rensselaer, N.Y.) and new cell lines from vertebral bone metastases were isolated and cultured. The resulting NPK-CAG^{YFP} bone cell lines (i.e., the NPK bone cells used herein) displayed >90% penetrance of metastasis to bone and to other tissues. The genotypes of the NPK bone cells and their derivatives were confirmed using a commercial source (Transnetyx, Inc, Memphis, Tenn., USA) and the cells were tested multi-species mycoplasma test using a nested PCR assay (Mycoplasma Detection Kit, Cat #MP70114, Fisher). Cell line stocks were established at passage 5 and used for experimental assays within 3 passages following thawing.

vii) Functional Analyses in Cell Culture and In Vivo

In addition to the mouse NPK bone cells (as above), functional validation studies were performed using PC3 human prostate cancer cell lines, which were derived from a human bone metastasis. PC3 cells were purchased from and authenticated by ATCC (American Type Culture Collection) using STR profiling, and grown in RPMI media supplemented with 10% FBS (ThermoFisher, Bridgewater, N.J., USA). Since the NPK bone cells were generated from NPK-CAG^{YFP} mice, they express YFP and can be tracked via ex vivo fluorescent imaging. To generate PC3 cells that can be monitored by in vivo bioluminescence as well as ex vivo fluorescent imaging, cells were engineered to express both luciferase and GFP using the pHAGE PGK-GFP-IRES-LUC-W lentiviral vector (Addgene, plasmid number 46793), which are herein referred to as PC3-Luc-GFP cells.

Lentiviruses were generated in HEK-293 cells (ATCC, Manassas, Va., USA), using second generation packaging vectors (psPAX2 and pMD2.G (Addgene, Cambridge, Mass., USA)). For shRNA-mediated silencing, a minimum of two independent shRNA clones were used for each gene using the pLKO.1 lentiviral vector system following manufacturer's instructions (Sigma-Aldrich, Allentown, Pa., USA). As a control, a non-targeting pLKO.1 lentiviral vector (SHC002, Sigma-Aldrich) was used. The sequences for all mouse and human shRNA used in this study are provided in Table S5.

Colony formation assays were performed by plating NPK-bone cells (200 cells/well) or PC3-Luc-GFP cells (1000 cells/well) in 6-well tissue culture plates. Two weeks after plating, colonies were visualized by staining with crystal violet and quantified using ImageJ software (obtained from https://imagej.nih.gov/ij/). Cell culture assays were done in triplicate and with a minimum of 2 independent biological replicates.

Allograft and xenograft assays were performed according to protocols approved by the Institutional Animal Care and Use Committee (IACUC) at Columbia University Irving Medical Center. For intracardiac metastasis assays, mouse NPK bone cells (1×10⁵ cells in 100 μl of PBS) were injected percutaneously into the heart's left ventricle of immunodeficient NCr nude mice (male, Taconic, Rensselaer, N.Y., USA). Mice were monitored daily and euthanized by 12-14 days after injection or sooner if their body condition score was <1.5 (as above). The NPK bone cells express YFP, allowing for direct visualization and quantification of metastases using a fluorescence microscope (as described above, and see FIG. S7).

For monitoring tumor growth subcutaneously, PC3-Luc-GFP cells (3×10⁶ cells in 100 μl of 50% Matrigel, Fisher Scientific) were injected into the left flank of male NOD-SCID mice (NOD.CB17-Prkdc^{scid}/J, Strain 001303, Jackson Laboratories). Tumor size was measured by caliper three times a week for up to eight weeks, and tumor volume estimated using the formula [Volume=(width)²×length/2]. At the time of euthanasia, all tumors were weighed and harvested as described above. For monitoring tumor growth in bone, PC3-Luc-GFP cells (1.5×10⁶ cells in 20 μl of PBS) were injected into the tibia. Briefly, a small longitudinal skin incision was made across the knee capsule and the tip of a scalpel was used to drill a hole into which the cells (or PBS alone) were injected in a volume of 20 μL. Sterile surgical bone wax (QuickMedical, Issaquah, Wash., USA) was then used to seal the hole, which was flushed with sterile PBS and the skin closed with wound clips.

Tumor growth was monitored bi-weekly for 8-10 weeks by bioluminescence imaging using an IVIS Spectrum Optical Imaging System (Perkin Elmer, Waltham, Mass.). Ten minutes prior to imaging, mice were injected intraperitoneally with 150 mg/kg D-luciferin (Perkin Elmer). Images were generated and quantified using Living Image Software (Perkin Elmer). Micro-computed tomography (CT) images of freshly dissected tibiae were also collected using a Perkin-Elmer Quantum FX micro-CT Imaging System.

viii) Statistical Analysis

Statistical analyses were performed using a two-sample two-tailed Welch t-test (for differential expression analysis), two-sample one-tailed Welch t-test (for comparison of MYC activity, META-55, and META-16 activity levels between SU2C and TCGA patient cohorts), one-way ANOVA, two-way ANOVA with multiple comparison testing, X² test, and Fisher's exact test as appropriate and indicated in each figure legend. GraphPad Prism software (Version 6.0) and R-studio 0.99.902, R v3.3.0 were used for statistical calculations and data visualization. Gene set enrichment analysis (GSEA) was performed, where NES and p-value were estimated using 1,000 gene or pathway permutations, as appropriate. For single-sample (i.e., single-patient) analysis, data were scaled (i.e., z-scored) on a gene level, so that a set of z-scores for each patient defined a “single-sample signature.” Subsequently, to estimate activity levels (e.g., for META-16, META-55, MYC genes and the like) in each sample, GSEA was utilized, where each “single-sample signature” was considered as a reference and genes of interest as a query gene set. To compare expression levels of META-16 gene signature across different metastatic sites, Gene Set Variation Analysis (GSVA) was performed, implemented as GSVA package (Bioconductor) in R.

Cox proportional hazards model and Kaplan-Meier survival analysis were done with the sure and coxph functions from survcomp package (Bioconductor) or using GraphPad Prism software. Radiographic evidence of metastatic disease was the primary endpoint for survival analysis on human validation cohorts. Statistical significance was estimated with Wald test and log-rank test, respectively. For Kaplan-Meier survival analysis, hierarchical consensus clustering was done on the expression levels of the META-55 and META-16 genes, which clustered patients into two groups: one group with high gene expression and one group with low gene expression for either META-55 or META-16 gene groups. Time to distant metastasis from radical prostatectomy was modeled using Cox proportional hazards model with and without adjusting for age, pathological Gleason score/grade at diagnosis, pre-PSA, seminal vesicle invasion (SVI), lymph node invasion (LNI), and extra-prostatic extension (EPE).

To evaluate the non-random ability of candidate genes (META-16 or META-55) to distinguish primary tumors in the TCGA cohort from the CRPC metastatic samples in the SU2C cohort (FIG. S9D), a random (equally sized, n=16 or n=55) group of genes was selected, and their estimated activity levels were compared between TCGA and SU2C cohorts using two-sample one-tailed Welch t-test. This random model procedure was repeated 10,000 times and two-sample one-tailed Welch t-test p-values from all iterations were used to build a Null model. The empirical p-value was then estimated as a number of times two-sample one-tailed Welch t-test p-values for a random group of 16 (or 55) genes reached or outperformed our original two-sample one-tailed Welch t-test p-value for the identified genes. In all cases, p-values represented with asterisk as follows: * for p-value<0.05, ** for p-value<0.01, *** for p-value<0.001, and **** for p-value<0.0001.

### Example 2

### A. Highly Penetrant Mouse Model of Bone Metastasis

As described in Example 1, reasoning that a challenge in identifying bone metastases is their detection, NPK mice were crossed with an enhanced fluorescence reporter allele (R26R-CAG-^{LSL-EYFP/+}) to generate NPK^{EYFP} mice (FIGS. 1A, 7A). As discussed above, these mice utilize an inducible Cre driven by the Nkx3.1 promoter Nkx3.1^{CreERT2/+}) to achieve temporal- and spatial-regulation of gene recombination of Pten^{flox/flox} and Kras^{LSL-G12D/+}, specifically in luminal prostatic cells, as well as activation of R26R-CAG-^{LSL-EYFP/+} for linage tracing by YFP, which permits fluorescent visualization of tumors and metastases (FIGS. 1A, 7A).

NPK^{EYFP} mice develop highly penetrant metastasis as evident by ex vivo YFP fluorescence as well as YFP immunostaining (=106), which is not seen in not control (un-induced) NPK^{EYFP} mice (n=3) or non-metastatic NP-CAG^{YFP} mice (for Nkx3.1^{CreERT2/+}; Pten^{flox/flox}; R26R-CAG-^{LSL-EYFP/+}) (n=35) (FIGS. 7A-C, FIG. 15, Table S1).

A high percentage of NPK^{EYFP} mice (n=47/106) display fluorescence in the bones, indicative of bone metastasis (44%; n=47/106) (FIGS. 1B, 7, SIB, Table S1). Ex vivo fluorescence was evident in the spine (n=32/47), pelvis (n=18/47), femur (n=22/47), tibia (n=9/47), and humerus (n=9/47) (FIG. 7C, FIG. 15H, Table S1), which are frequent sites of bone metastasis in human prostate cancer.

Longitudinal analyses revealed micro-metastases in bone by 3 months after tumor induction (FIG. 15I), similar to when DTCs are first detected. Micro-metastases occurred earlier and were more prevalent in bone than lung (FIGS. 15I-J). Immunostaining revealed YFP-expressing cells in bone that express several markers that are expressed in primary tumors, including Ki67 and the luminal cytokeratin, Ck8 (FIGS. 7B-C, Table S1). This confirmed their origin from lineage-marked prostatic cells.

Overall, there were few discernible differences in NPK^{EYFP} mice that developed bone metastases (n=47/106) versus those that did not (n=59/106). However, those with bone metastases had a significantly augmented metastatic phenotype, with an average of >80 lung, >40 liver, and at least 1 brain metastasis; whereas those without bone metastases had relatively few lung, and few if any liver or brain metastases (P<0.001; FIG. 7, Table S1).

Similar to primary tumors and lung metastases, bone metastatic cells were found to express androgen receptor (AR) protein and AR activity but negligible levels of synaptophysin, a marker of NEPC and low levels of NEPC activity (FIGS. 1B-1E, 7B-E, FIG. 16). Notably, surgical castration did not appear to affect median survival, incidence of bone or other metastases, or expression of activity levels of AR or NEPC when comparing castrated (n=22) and non-castrated (n=106) mice (FIGS. 1D-E, 7D-E, FIG. 16; Table S1). Gene set enrichment analyses (GSEA) comparing bone metastases with primary tumors from castrated (n=6) and non-castrated (n=13) mice revealed significant similarity (P<0.001; FIG. 16; Table S2).

B. Conservation of Bone Metastases in NPK^{EYFP} with Human Prostate Cancer

To investigate the cell-intrinsic molecular phenotype of the bone metastatic cells compared with the primary tumor cells, RNA sequencing was performed on primary tumors (n=19) and bone (n=12), lung (n=11), liver (n=5), brain (n=3) and lymph nodes (n=4) metastases from 16 independent NPK^{EYFP} mice (Table S2). Principal component analyses showed that bone metastases clustered separately from primary tumors and other metastases (FIGS. 2A, 8A).

To evaluate conservation with human prostate cancer, a mouse “bone metastasis signature” was defined by comparing bone metastases (n=12) with primary tumors (n=19) (Table S2). Cross-species GSEA comparing this signature with an analogous signature of human bone biopsies (n=10) and primary tumors (n=19) from patients with metastatic prostate cancer (Balk cohort, Table S3) revealed significant enrichment (FIGS. 2B, 8B).

Whole-exome sequencing (WES) of matched sets of primary tumors, bone and lung metastases, as well as control DNA (tail) from the five NPK^{EYFP} mice did not identify significant somatic mutations or alterations of tumor suppressors or oncogenes, similar to other genetically engineered mouse models (GEMMs). Nonetheless, WES permitted reconstruction of evolutionary trees for dominant clones in the primary tumor, bone and lung metastases (FIG. 2C-D, 8C-D, Table S4). Phylogenetic analysis revealed that the common recent ancestor of tumor and bone metastasis preceded the common recent ancestor of tumor and lung metastasis in four of five mice (P=1.6×10⁻⁷; FIGS. 2C-D), suggesting that an earlier metastatic clone seeded the bone metastasis, while lung metastases were derived from a later clone, consistent with the present finding that micro-metastases in bone arise earlier than in lung (FIG. 15I-J).

This was consistent with the finding herein that micro-metastases in bone arise earlier than in lung (FIGS. 15I-J). Inference of copy number variations (CNVs from the WES data also revealed few significant gains or losses in primary tumors or metastases (Table S4). Nonetheless, informative CNV events reflect the history deduced by the SNV analyses, thus further supporting the inferred revolutionary hierarchy (FIGS. 2C, 8C).

Among the few significant CNVs, Kras was amplified in tumors of all five NPK^{EYFP} mice; analogous to other Kras-driven GEMMs, this spanned the entire chromosome 6 (FIG. 17; Table S4). In human prostate cancer, KRAS copy number gains occur in 2% of primary tumors (based on The Cancer Genome Atlas Prostate Adenocarcinoma, TCGA, n=497), but 20% of metastases (based on the Stand Up to Cancer SU2C, n=429) (FIG. 17, Table S3). Thus, the NPK^{EYFP} mice are shown to model key features of human prostate cancer metastasis.

### C. Activation of MYC Pathway is Cell-Intrinsic to Bone Metastasis in NPK^{EYFP} Mice

Single-cell RNA sequencing was performed on matched samples from primary tumor and bone metastases (FIG. 9, Table S5). As visualized using uniform manifold approximation and projection (UMAP), the primary tumor cells (black) separated into a major (83%) and smaller (17%) group; the bone metastatic cells (dark grey) separated into a major group (77%) projecting far from primary tumor cells and a smaller group (23%) projecting close to the tumor cells (FIG. 9A).

Unsupervised clustering revealed that the larger group of bone cells were Cd45+, while the smaller group were YFP+ (P<10⁻²³⁴; FIGS. 3B-C). This smaller group was highly enriched for Ck8 expression and AR activity (Ck8, P=3.5×10⁻³¹⁵; AR P=2.7×10⁻¹³⁴; FIG. 3C), consistent with immunostaining results (FIGS. 7B-C). Thus, it was inferred that the YFP+(Cd45 negative) bone cells projecting close to the major group of primary tumor cells are metastatic cells, whereas the Cd45+(YFP negative) bone cells are benign resident bone cells. In subsequent analyses, focus was on the major primary tumor cells (black) and the YFP-expressing bone metastatic cells (dark grey), hereinafter called, the “primary tumor cells” and “bone metastatic cells” respectively (FIG. 9D).

Since the “bone metastasis signature” defined by bulk RNA sequencing invariably includes non-tumor cells, in certain embodiments tumor cell-intrinsic features can also be distinguished by projecting this signature on one from the single-cell bone metastatic and primary tumor cells (hereafter called the “single-cell bone metastasis signature”; Table S5). At the gene level, the “bone metastasis signature” was significantly enriched in the “single-cell bone metastasis signature” (P<1×10⁻³²⁴, FIG. 9E). Similarly, differentially regulated pathways in the “bone metastasis signature” were significantly enriched with those in the “single-cell bone metastasis signature” (P<0.001; Table S5). Furthermore, pathway analyses using a bulk RNA signature comparing bone metastases with normal bone (Table S2) revealed significant enrichment with the “single-cell bone metastasis signature” (P<0.001; FIG. 9G). Together, these findings indicate that cell-intrinsic features of bone metastatic cells drive the “bone metastasis signature.”

Among leading-edge pathways enriched between the bulk and single-cell bone metastases signatures, in certain embodiments the most significant was the Hallmarks MYC pathway. In certain embodiments, Hallmark MYC pathway genes were positively enriched in the “single-cell bone metastatic signature” (P<0.001; FIG. 9H) but downregulated in a single-cell gene signature comparing the benign resident bone cells with primary tumor cells (P=0.002; FIG. 9I, Table S5). These analyses implicate MYC pathway activation as a principal cell-intrinsic feature of bone metastases in NPK^{EYFP} mice.

### D. Co-Activation of MYC and RAS in Prostate Cancer Metastasis

Cross-species GSEA comparing pathways enriched in the mouse single-cell bone metastasis signature with those enriched in a human signature comprised of primary tumors (n=19) and bone biopsies (n=19) (Balk; Table S3) revealed significant similarity (P<0.001; FIG. 18A). In certain embodiments, the most significant was the Hallmarks MYC pathway (P<0.001; FIG. 18B). In certain embodiments, MYC activation was further evident by comparing pathways enriched in the mouse “bone metastasis signature” with this Balk signature, which is comprised of tumors and bone biopsies from patients living with metastatic prostate cancer (Table S3), and a second human signature comprised of primary prostate tumors (n=14) and bone metastases (n=20) from patients who had died of metastatic prostate cancer (FHCRC, Table S3). Results showed that pathways upregulated in the Balk and FHCRC cohorts were highly enriched compared with those of the mouse bone metastasis signature (P<0.001; FIG. 18A). Stouffer integration to identify pathways significantly enriched among all three mouse and human signatures revealed, in certain embodiments, the Hallmarks MYC pathway as the most significant (FIG. 18B). In certain embodiments, these mouse and human signatures were also significantly enriched with canonical MYC targets (Dang, P<0.001) and oncogenic MYC targets (Sabo; P<0.003, FIG. 18C).

Consistent with the known up-regulation of MYC in human prostate cancer, immunostaining showed robust expression of MYC in human bone metastases from patients with mCRPC (n=12; FIG. 10C). As observed for KRAS, copy number gains in MYC are more prevalent in human prostate cancer metastases (70% in SU2C) compared with primary tumors (31% in TCGA, FIG. 10). Although MYC copy number gains in NPK^{EYFP} mice was not observed (FIG. 9), Myc mRNA was found to be upregulated in bone metastases relative to primary tumors and lung metastases (Table S2).

To investigate further MYC pathway activation in human prostate cancer metastasis, single-sample GSEA was performed to estimate Hallmarks MYC pathway activity levels (hereinafter, “MYC activity”) in individual cases of metastases from SU2C (n=270) and primary tumors from TCGA (n=497), which showed strong enrichment in metastases (FIG. 10D). The overall distribution of MYC activity was found to be significantly greater in metastases compared with primary tumors (P=1×10⁻¹⁴; FIG. 4D), although MYC did not appear to be preferentially activated in bone metastasis relative to other metastatic sites (FIG. 17).

In NPK^{EYFP} mice, strong enrichment of Myc activity in metastases relative to primary tumors was observed (P=3×10⁻⁹; FIG. 10E); however, unlike human prostate cancer, Myc activation was specific for bone metastases relative to other metastatic sites (P=6.1×10⁻¹⁰; FIG. 17). One major difference between the human prostate cancer and mouse cohorts is that the SU2C cohort are from mCRPC, whereas the mouse cohort is androgen-intact. Notably, robust MYC immunostaining in mCRPC was observed (n=34).

Indeed, despite strong overall similarity of their molecular profiles (FIG. 16G), Myc pathway activity was observed to be significantly up-regulated in castrated versus non-castrated NPK^{EYFP} mice in the bone metastases as well as primary tumors and other metastatic sites (P<0.01; FIG. 17). It is conceivable that MYC is already activated in mCRPC in SU2C, thereby obscuring activation in bone metastases. Notably, while analyses of NPK^{EYFP} mice allows investigation in androgen-intact and -deprived contexts, we are unaware of a human cohort of bone and other metastases from castrated and non-castrated patients that would allow direct comparison of MYC in these contexts.

RAS pathway activation in human and mouse metastasis was analyzed based on the expression of seven genes (i.e., PTPN11, KRAS, NRAS, BRAF, RAF1, SPRY1, SPRY2) associated with RAS/RAF signaling as described in (hereafter called “RAS activation”). Similar to MYC activation (FIG. 10D), strong enrichment of RAS activation in metastases from SU2C was observed compared with primary tumors from TCGA (P=7.4×10⁻⁶⁷; FIG. 10F) and, like MYC, RAS activation was not preferential to bone metastasis (FIG. 17). However, in NPK^{EYFP} mice, Ras activation was specific to bone metastases (P=1×10⁻⁴; FIG. 10G; FIG. 17).

Activation of MYC and RAS are well-correlated in human prostate cancer (Spearman correlation, rho 0.37, P<0.001; FIG. 17I), particularly in advanced tumors (Gleason Grades 8-10) and metastases. Case by case analyses revealed up-regulation of MYC activity in 190 of 497 tumors from TCGA. 80 of these (16%) have co-activation of RAS, whereas 193 of 270 metastases in SU2C have upregulated MYC and 177 of these (66%) have co-activation of RAS (P<2.2×10¹⁶; FIG. 10H). In NPK^{EYFP} mice, activation of Myc and Ras are strongly correlated (Spearman correlation, rho 0.67, P=6.6×10⁻⁴, FIG. 17H), particularly in bone. Further, co-activation of MYC and RAS occurred in only 1 of 13 (8%) primary tumors but 9/10 (90%) of bone metastases in NPK^{EYFP} mice (P=1×10⁻⁴; FIG. 17J). Therefore, co-activation of MYC and RAS is significantly associated with prostate cancer metastasis and effectively modeled in NPK^{EYFP} mice.

### E. MYC is Necessary but not Sufficient for Bone Metastasis

The function of MYC for bone metastasis was investigated using an in vivo allograft model derived from NPK^{EYFP} mice (FIG. 5, FIG. 11). Intracardiac injection of NPK^{EYFP} bone cells, but not control cells from non-metastatic NP tumors, results in metastases to bone, as well as lung and other tissues (FIG. 11; FIG. 19). Silencing Myc using two different shRNAs (shMyc#1 or shMyc#2), but not the control shRNA (shControl), resulted in significant reduction in bone metastases, while not abrogating cellular viability (FIGS. 11B-C, FIG. 19A). Silencing Myc inhibited metastasis in each type of bone (spine, pelvis, femur, tibia and humerus), whereas lung metastases were not significantly affected (n=8, FIGS. 11C-D). While bones from mice injected with control NPK^{EYFP} bone cells displayed YFP-marked bone metastases with robust Myc expression, bones from mice injected with Myc-silenced NPK^{EYFP} bone cells had fewer or no YFP-marked bone metastases and low expression of Myc (FIGS. 11D-E).

MYC is highly expressed in human PC3 cells, which were derived from a bone metastasis, and grow in bone when implanted orthotopically. Therefore, MYC function for tumor growth in bone was examined using PC3 cells engineered to express luciferase and green fluorescent protein (GFP) (herein called, “PC3-Luc-GFP cells”) (FIG. 20). Silencing MYC in PC3-Luc-GFP cells using two different shRNAs (shMYC#1 or shMYC#2), but not the control shRNA (shControl), was found to inhibit tumor growth when implanted into tibia, while not completely abrogating cellular viability (P<0.0001; FIGS. 20B-F). Tibiae of mice implanted with control PC3-Luc-GFP cells (shControl) displayed large YFP-expressing tumors, which were not observed in mice implanted with MYC-silenced cells (FIG. 20G).

Since previous studies of Myc in other prostate cancer mouse models reported no or low incidence of bone metastasis, NPK^{EYFP} mice were crossed with the hi-MYC transgene to generate a series of GEMMs having activation of neither Myc nor Ras (NP^{EYFP}); activation of either Myc (NPM^{EYFP}) or Ras (NPK^{EYFP}); or co-activation of Myc and Ras (NP^{EYFP}) (FIG. 20).

In certain embodiments, while NPM^{EYFP} mice developed large prostate tumors and lung metastasis, they did not develop bone metastasis or lethal prostate cancer (n=23; FIGS. 12A-D). Further, while NPKM^{EYFP} mice developed both lung and bone metastasis, their incidence of bone metastasis and overall survival were similar to NPK^{EYFP} mice (n=10; FIGS. 12A-D). As above, Myc activity was found to be significantly greater in bone metastases compared with primary tumors from NPK^{EYFP} mice (P=3.2×10⁻⁹, FIG. 12E and see FIG. 10E). However, Myc activity in primary tumors of NPK^{EYFP} mice were found to be significantly higher than in primary tumors of the NPK^{EYFP} mice (P=0.015), but comparable to bone metastases (FIG. 12E). Ras pathway activity was also found to be significantly higher in NPK^{EYFP} bone metastases relative to the primary tumors from either NPK^{EYFP} or NPK^{EYFP} mice (P=0.0014, FIG. 12F). These findings show that MYC is necessary but not sufficient for bone metastasis, and suggest it requires collaboration with RAS activation for bone metastasis.

### F. META-16: A Human Gene Signature Prognostic for Time to Metastasis and Treatment Response

Interrogation of the PROMOTE cohort was used to identify a gene signature associated with co-activation of MYC and RAS. The PROMOTE cohort (for PROstate Cancer Medically Optimized Genome-Enhanced ThErapy) is comprised of metastatic biopsies from patients with mCRPC (n=77), the majority of which are bone (n=55) (FIGS. 13A-B, Table S3). Genome-wide correlation based on PROMOTE identified 559 genes positively correlated with MYC expression (“PROMOTE-559”; Spearman rho>0.5, FDR P<0.0001, FIGS. 13A-B); 517 of these (93%) were also correlated with RAS activation.

Interrogation of bone metastasis signatures with PROMOTE-559 revealed significant enrichment in both the mouse (P<0.001, FIG. 7B, FIG. 13A) and human (P<0.001, FIG. 6B, FIG. 13B) signatures. Integration of leading-edge genes from the mouse (121) and human (154) signatures identified 55 genes (“META-55”; FIG. 13B); 52 of these (95%) were also correlated with RAS activation.

To prioritize the META-55 genes for association with metastasis, a univariable Cox proportional hazards model was used, based on metastasis-free survival for 336 patients in TCGA that had reported “time to metastasis” (13 developed metastasis; Table S3). This identified 16 genes (“META-16”) with significant association to metastasis-free survival (P<1×10⁻⁷; FIG. 21C). All 16 (100%) were found to correlate with RAS activation.

Since META-16 consistently outperformed META-55, subsequent analyses focused on this signature; however, findings here include both signatures (FIGS. 21-24). Discovery of META-16 was improved by cross-species interrogation, since analyses of only human signatures identified 48.5% significantly associated to time to metastasis (P<0.01) whereas analyses of both mouse and human signatures identified 74.5% (P<0.01), which is a significant improvement (P=0.0021).

Analysis of single-cell sequencing data revealed significant enrichment of META-16 in bone metastatic versus primary tumor cells (P=2.5×10⁻²⁸⁹) and strong correlation with Myc activation (Spearman correlation P=2.2×10⁻¹⁶; FIGS. 13C-D, FIG. 21). GSEA showed enrichment of META-16 in the single-cell bone metastasis signature (P=0.019; FIG. 13E, but not the single-cell signature based on tumor versus the benign resident bone cells.

Expression of META-16 genes was up-regulated in human bone metastases relative to primary prostate tumors (P<0.05, FIG. 23A), while silencing MYC in human (PC3-Luc-GFP) or mouse (NP K bone) metastatic prostate cancer cells resulted in reduced their expression (P=0.034 for PC3, P<0.01 for NPK, FIG. 23B-C). Notably, META-16 expression in SU2C is significantly higher in patients with MYC amplification (P=0.0006) but not PTEN deletion (P=0.11). Although META-16 includes several genes located near and potentially co-amplified with MYC on human chromosome 8q, META-16 performs equally well without these genes (P=3.7×10⁻¹⁵¹ and FIG. 22).

META-16 is strikingly enriched across human prostate cancer metastases from various tissue sites although, as for MYC, not exclusively in bone metastases. Single-sample GSEA on each tumor from TCGA (n=497) and each metastasis from SU2C (n=270) showed strong enrichment of META-16, particularly in metastases (FIG. 22). The overall distributions between the TCGA and SU2C revealed significant up-regulation of META-16 in metastases compared to primary tumors (P<10⁻¹²⁵, FIG. 13F, FIG. 22). Individual genes META-16 were up-regulated across each metastasis versus each primary tumor (FIG. 13G, FIG. 22). Comparing activity levels of an arbitrary, equally sized (n=16) group of genes showed that the ability of META-16 to distinguish primary tumors from metastases was non-random (P=0.003, FIG. 21D).

To ask whether META-16 is significantly associated with risk of metastasis, we used two independent prostatectomy cohorts with extensive clinical outcome data (MAYO⁴³ and JHMI⁴⁴, FIGS. 14A-C, FIG. 24, Table S3). Patients in MAYO (n=235) had undergone radical prostatectomy between 2000 and 2006 with a median follow up of 7 years; 76 patients developed metastasis. Patients in JHMI (n=260) had undergone radical prostatectomy between 1992 and 2010 with a median follow up of 9 years; 99 patients developed metastasis.

To test association of META-16 with metastasis-free survival, hierarchical clustering was performed to group patients with low or high levels of combined META-16 expression (FIGS. 24A-B). In both cohorts, Kaplan-Meier survival analyses demonstrated that patients with high expression of META-16 have a shorter time to metastasis than those with low expression (P<0.0001;

FIGS. 14A-B, FIGS. 24C-D). A multivariable Cox proportional hazards model, adjusted for age, pathological Gleason score/grade at diagnosis, pre-PSA, seminal vesicle invasion SVI, lymph node invasion LNI, and extra-prostatic extension, showed that the ability of META-16 to predict metastasis-free survival is not affected by those variables and is significantly associated with metastasis-free survival (MAYO, P=0.0001; JHMI, P=0.0006) compared to prostate-cancer specific mortality (MAYO, P=0.05; JHMI, P=0.15; FIG. 14C, FIG. 24E.

SU2C includes 75 patients with detailed clinical data regarding treatment-associated survival (i.e., time from the start of treatment with androgen signaling inhibitors, ARSIs, to death or last follow-up), as well as 56 patients with detailed information about treatment-associated disease progression (i.e., time on treatment with ARSIs). To ask whether META-16 is associated with treatment response, these patients were grouped into low or high levels of combined META-16 expression. Subsequent Kaplan-Meier survival analyses demonstrated that patients with high META-16 expression have a shorter time to treatment-associated death (P=9.2×10⁻⁴) and a shorter time to treatment-associated disease progression (P=0.018; FIGS. 14D-E). Consistent with our observations that META-16 is strongly correlated with MYC activity, when we grouped these same patients based on high versus low levels of MYC activity, we found that those with high activity had a shorter time to treatment-associated survival (P=0.0013) as well as shorter time to treatment-associated disease progression (P=0.0014, FIGS. 24F-G). Therefore, META-16 and MYC activity may have predictive significance for response to treatment with anti-androgens in advanced prostate cancer.

### G. Methods

### i) Genetically Engineered Mouse Model of Bone Metastasis

All experiments using animals were performed according to protocols approved by the Institutional Animal Care and Use Committee (IACUC) at Columbia University Irving Medical Center. All mice were housed in pathogen-free barrier conditions under 12-hour light/dark cycles and with temperature and humidity set-points at 20-25° C. and 30-70%, respectively. Since the focus of the experiments was prostate cancer, only male mice were used.

Nkx3.1^{CreERT2/+}; Pten^{flox/flox}; Kras^{lsl-G12D/+} (NPK) mice were crossed with the Rosa-CAG-LSL-EYFP-WPRE reporter allele to obtain the experimental Nkx3.1^{CreERT2/+}; Pten^{flox/flox}; Kras^{lsl-G12D/+} R26R-CAG-^{LSL-EYFP/+} (NPK^{EYFP}) mice and the control (non-metastatic) Nkx3.1^{CreERT2/+}; Pten^{flox/flox}; Kras^{+/+}; R26R-CAG-^{LSL-EYFP/+} (NP^{EYFP}) mice. The Hi-MYC allele (FVB-Tg(ARR2/Pbsn-MYC) was crossed with the NPK^{EYFP} mice to obtain the NP-Hi-MYC^{EYFP} (NPM^{EYFP}) and NPK-Hi-HMYC^{EYFP} (NPM^{EYFP}) mice. NPK mice have been maintained in our laboratory on a predominantly C57BL/6 background; the Rosa-CAG-LSL-EYFP-WPRE mice were obtained from Jackson Laboratories on a C57BL/6 background (Stock No: 007903); and the Hi-MYC mice were obtained from the NCI mouse repository on an FVB background (Stock No: 01XK8). Of note was the significant increase in median survival of the NPK^{EYFP} mice compared with past reports (4.7 months compared with 3.1 months, P<0.0001). This difference can be attributed to the low level expression of the first generation YFP reporter allele in the previous NPK mice, which required that homozygotes be analyzed. In contrast, in the current NPK^{EYFP} mice, heterozygotes of the second generation Rosa-CAG-LSL-EYFP-WPRE allele were analyzed.

As with Example 1, all studies were done using littermates that were genotyped prior to tumor induction; since the focus of the study was prostate cancer, only male mice were used. Mice were induced to form tumors at 2-3 months of age by administration of tamoxifen (Sigma-Aldrich, Allentown, Pa., USA) using 100 mg/kg (in corn oil) once daily for 4 consecutive days. Control (non-tumor induced) NPK^{EYFP} mice were delivered only the vehicle (corn oil). The primary survival cohort (n=106) were euthanized when their body condition score was <1.5, or when they experienced body weight loss ≥20% or signs of distress, such as difficulty breathing or bladder obstruction. A second survival cohort (n=22) underwent surgical castration 1 month after tumor induction. The longitudinal cohort (n=26) was euthanized at the specific time points following tumor induction as indicated.

At sacrifice, YFP-positive prostatic tumors and metastases were visualized and quantified by ex vivo fluorescence using the same equipment as in Example 1; and histological and immunohistochemical analyses, the same processes were used. For the RNA sequencing analysis, the same process as that of Example 1 was used, except that reads for each sample were aligned to the mm9 mouse genome using Tophat.(v1.1.0).

### ii) Whole Exome Sequencing Analysis of Mouse Tumors and Bone Metastases

Whole exome sequencing (WES) was done on matched trios of primary tumors, lung metastases, and bone metastases, as well as tails (as a control) from five independent NPK-CAG^{EYFP} mice. Genomic DNA was isolated sequenced in the same steps as in Example 1. The resulting average sequencing depth was more than 80×, and reads were mapped to the mouse mm10 genome build using bwa (v 0.0.17). Substitutions and indels were called using MuTect2 (v 4.0.4) with default parameters; only variants with a mutant allele frequency of 5% or greater in tumors and 0% in normal tail were included for further analysis. The variant read count cutoff was 5 or more in tumor and depth was 20 or more in normal tail. A list of single nucleotide variants is provided in Table S5.

Evolutionary trees were reconstructed using somatic mutations (i.e., substitutions and indels), in the same manner as in Example 1. The significance of the phylogeny of the evolutionary tree was tested using bootstrap test, as described therein. Representative combined phylogeny was then constructed reflecting consistent evolutionary patterns across all trees and its meta-analysis p-value was calculated using Fisher's method through combining bootstrap-derived p-values from individual trees.

iii) Single-Cell RNA Sequencing Analyses of Mouse Tumors and Bone Metastases

Single-cell RNA sequencing was done on freshly dissected prostate tumor and bone metastases from NPK-CAG^{EYFP} mice in two independent experiments using the 10× Genomics Chromium platform. The tissues were prepared in the same manner as in Example 1, from the enzymatically digested step to the dilution 4-fold in cold PBS, centrifugation and resuspension for cell counting and viability analysis.

Cells were counted using a Countess II Automated Cell Counter (ThermoFisher) and 10,000 cells with over 70% viability were loaded into a 10× Genomics Chromium Controller for capture and barcoding following the 10× Genomics Single Cell Protocol, as described by the manufacturer (10× Genomics, Pleasanton, Calif., USA), with subsequent RNA sequencing using Illumina NovaSeq. Reads were mapped to the mouse mm9 genome and processed with the CellRanger pipeline. Data are provided in Table S5.

Single-cell RNA-seq raw counts were normalized and the variance was stabilized using DESeq2 package (Bioconductor) in R-studio 0.99.902, R v3.3.0 (The R Foundation for Statistical Computing, ISBN 3-900051-07-0). The uniform manifold approximation and projection (UMAP) dimensionality reduction technique implemented in Python was used to cluster primary and metastatic single-cell RNA sequencing data. UMAP visualizations were constructed as described; the code for visualization is available at https://github.com/simslab.

### iv) Description of Human Patient Cohorts

All studies using human tissue specimens were, as with Example 1, performed according to protocols approved by the Human Research Protection Office and Institutional Review Board (IRB) at the respective institutions. Published human patient cohorts used for discovery (i.e., the Balk, FHCRC, and PROMOTE cohorts) or validation (i.e., the TCGA, SU2C, JMHI, and MAYO cohorts) are described in Table S2.

The subset of SU2C patients used herein (n=270) used polyA+ RNA isolation for transcriptomic library preparation. Among these, clinical outcome data was available for 75 patients based on treatment-associated survival analysis (41 patients died) and 57 patients based on treatment-associated disease progression analysis (47 patients experienced disease-progression related events). Treatment-associated survival was defined as time between start of ARSIs treatment and death or follow-up, and treatment-associated progression was defined as time on ARSIs treatment.

Unpublished cohorts used anonymized human tissue specimens from Columbia University Irving Medical Center (CUIMC) or Johns Hopkins Hospital (JHH); all patients were consented before inclusion. 859 The CUIMC cohort, which was used for analysis of RNA expression, was comprised of 5 bone metastatic resections and 10 primary prostate cancer tumors (Gleason score 9) from surgical resections of patients with advanced prostate cancer that had been banked in the Molecular Pathology Shared Resource of the Herbert Irving Comprehensive Cancer Center. RNA was extracted using miRNeasy mini kit (Qiagen) and qRT-PCR was done using the QuantiTect SYBR Green PCR kit (Qiagen, Germantown, Md.).

The JHH cohort, which was used for immunohistostaining, was comprised of 34 metastatic samples including 12 bone metastatic biopsies from patients diagnosed with advanced prostate cancer. The clinical features of the patients are summarized in the present disclosure. Immunohistochemistry was done using a rabbit monoclonal MYC antibody (Abeam, Cambridge, Mass.). Immunostaining was quantified using an H-score system obtained by multiplying staining intensity (0: no staining; 1: weak staining; 2: moderate staining; 3: intense staining) by the percentage (0-100) of cells showing that intensity (H-score range 0-300, with 0-100 considered low, 101-200 intermediate and 201-300 high).

For Functional Analyses in Cell-Based Models, and Statistics and Reproducibility, the same protocols were followed as in Example 1.

Supplementary Tables:

TABLE S1
Summary of the phenotypic analysis of NPK-CAG^{YFP} mouse cohort
Summary of the metastatic phenotype of the NPK-CAG^{YFP} prostate cancer mouse model

| | | Lung mets | | | | | | | | | | | | | |
|---|---|---|---|---|---|---|---|---|---|---|---|---|---|---|---|
| | | | | Low | | High | | | | Liver | | | | | |
| | | Total | | (<54) | | (>54) | | Lymph node mets | | mets | | Brain mets | | Bone mets | |
| | | # of | | # of | | # of | | # of | | # of | | # of | | # of | |
| Genotype | n | cases | % | cases | % | cases | % | cases | % | cases | % | cases | % | cases | % |
| NP-CAG^{YFP} | 25 | 0 | 0 | — | — | — | — | 0 | 0 | 0 | 0 | 0 | 0 | 0¹ | 0 |
| NPK-CAG^{YFP} | 106 | 105 | 99 | 66 | 62 | 40 | 38 | 106 | 100 | 72 | 68 | 43 | 41 | 47 | 44 |
| NPK-CAG^{YFP} | 3 | 0 | 0 | — | — | — | — | 0 | 0 | 0 | 0 | 0 | 0 | 0 | 0 |
| (Un-induced²) | | | | | | | | | | | | | | | |

¹In non-metastatic NP-CAG^{YFP} mice, all bones were examined checked in 7 mice
²Un-induced NPK-CAG^{YFP} control mice received corn-oil instead of tamoxifen

| | | Spine | | Pelvis | | Femur | | Tibia | | Humerus | |
|---|---|---|---|---|---|---|---|---|---|---|---|
| | | # of | | # of | | # of | | # of | | # of | |
| Genotype | n* | cases | % | cases | % | cases | % | cases | % | cases | % |
| NPK-CAG^{YFP} | 47 | 32 | 68 | 18 | 38 | 22 | 47 | 9 | 19 | 9 | 19 |

*Bone metastases were found in 47/106 mice in the NPK-CAG^{YFP} cohort

| | Intact vs Castrated | | | Low vs High Met Phenotype | | | | |
|---|---|---|---|---|---|---|---|---|
| | Intact | | | | High | | | |
| | # of | Castrated | p- | Low | # of | | Survival | |
| NPK-CAG^{YFP} mice¹ | cases | # of cases | value² | # of cases | cases | p-value² | Median | p-value² |
| W/out bone mets | 59 | 12 | ns | 51 | 8 | <0.0001 | 5.2 | 0.03 |
| With bone mets | 47 | 10 | | 15 | 32 | | 4.4 | |

*Includes intact (n = 106) and castrated (n = 22) NPK-CAG^{YFP} mice for a total n = 128.
¹Two-sided Fisher’s exact test
²Log-rank test

| | | | | Prostate Lobe | | | | | | Coat Color | | |
|---|---|---|---|---|---|---|---|---|---|---|---|---|
| NPK-CAG^{YFP} | Urinary Occlusion | | | | | p- | Weight Loss | | | | | p- |
| mice | No | Yes | p-value¹ | AP | DLP | value¹ | No | Yes | p-value¹ | Black | Agouti | value¹ |
| W/out bone mets | 28 | 30 | 0.02 | 25 | 18 | 0.02 | 16 | 40 | ns | 21 | 37 | ns |
| With bone mets | 31 | 12 | | 15 | 32 | | 10 | 29 | | 11 | 31 | |

¹Two-sided Fisher’s exact test

Table S2: Summary of the Human Prostate Cancer Expression Profiling Datasets Used in this Study

TABLE S2
Description of human datasets used in this study

| | | | | |
|---|---|---|---|---|
| Ref/name | Description and use in this study | n | Platform | Geo/Ref |

Discovery cohorts

| | | | | |
|---|---|---|---|---|
| Name: | Description: | | | |
| Balk | Bone metastases from CRPC obtained | 19 | Affymetrix | GEO: |
| | from bone marrow biopsies | | Human | GSE32269 |
| | Hormone treatment-naïve prostate tumors | 19 | Genome | |
| | isolated from frozen biopsies | | U133A Array | |
| | Use: | | | |
| | Cross-species discovery of conserved genes | | | |
| | and pathways associated with bone | | | |
| | metastasis | | | |
| Name: | Description: | | | |
| FHCRC | Bone metastases from CRPC obtained | 20 | Agilent 44K | GEO: |
| | from rapid autopsies | | whole human | GSE77930 |
| | Primary tumors from CRPC obtained from | 14 | genome | |
| | rapid autopsies | | expression | |
| | Use: | | oligonucleotide | |
| | Cross-species discovery of conserved pathways | | microarray | |
| | associated with bone metastasis | | | |
| Name: | Description: | | | |
| PROMOTE | Metastatic CRPC samples obtained from | 77 | Illumina HiSeq | dbGap: |
| | tissue biopsies (55 are bone mets) | | 2500W | phs001141.v1.p1 |
| | Use: | | | |
| | Discovery of genes that are correlated with | | | |
| | MYC expression in metastases | | | |

Validation cohorts

| | | | | |
|---|---|---|---|---|
| Name: | Description: | | | |
| TCGA | Surgical resection biospecimens from | 497 | Illumina HiSeq | TCGA Data |
| | prostate adenocarcinoma without prior | | 2000 W | Portal: |
| | treatment | | | https://portal.gdc.cancer.gov/ |
| | Use: | | | |
| | Validation of the levels of MYC activity and | | | |
| | expression of metastasis signatures in | | | |
| | primary tumors | | | |
| Name: | Description: | | | |
| SU2C | Bone or soft tissue tumor biopsies from | 270 | Illumina HiSeq | GitHub |
| | metastatic castration-resistant prostate | | 2500 | portal: https://github.com/ |
| | cancer (CRPC) obtained from fresh frozen | | | cBioPortal/datahub/tree/ |
| | needle biopsies | | | master/public/prad_su2c_2019 |
| | Use: | | | |
| | Validation of the levels of MYC activity and | | | |
| | expression of metastasis signatures in | | | |
| | metastases | | | |
| Name: | Description: | | | |
| JMHI | Retrospective case-cohort study of | 260 | Affymetrix | GEO: |
| | primary tumors of men at high-risk of | | Human Exon | GSE79957 |
| | recurrence | | 1.0 ST Array | |
| | Use: | | | |
| | Association of metastasis signatures with | | | |
| | clinical outcome | | | |
| Name: | Description: | | | |
| MAYO | Retrospective case-cohort study of | 235 | Affymetrix | GEO: |
| | primary tumors of men at high-risk of | | Human Exon | GSE62116 |
| | recurrence | | 1.0 ST Array | |
| | Use: | | | |
| | Association of metastases signatures with | | | |
| | clinical outcome | | | |

TABLE S3
Description of the human metastatic prostate cancer specimens used for MYC immunostaining
Clinical data for JHH human metastatic prostate cancer cases used for MYC immunostaining

| | MYC staining | | | | | | | | | |
|---|---|---|---|---|---|---|---|---|---|---|
| | | | | H- | | Age @ | Gleason | # of | Status | Metastasis |
| Patient #⁽¹⁾ | Strong | Moderate | Weak | score | Race² | DX³ | (scores)⁴ | treatments⁵ | at biopsy | Site⁶ |
| 1 | 30 | 40 | 10 | 180 | W | 47 | 8 | 11 | CRPC | Liver |
| | | | | | | | (4 + 4) | | | |
| 2 | 0 | 15 | 5 | 35 | W | 60 | 9 | 5 | CRPC | Lymph node |
| | | | | | | | (4 + 5) | | | |
| 3 | 10 | 10 | 10 | 60 | W | 54.5 | 7 | 6 | CRPC | Lymph node |
| | | | | | | | (3 + 4) | | | |
| 4 | 70 | 30 | 0 | 270 | W | 61 | 7 | 8 | CRPC | Liver |
| | | | | | | | (4 + 3) | | | |
| 5 | 5 | 5 | 0 | 25 | W | 51 | 8 | 9 | CRPC | Lymph node |
| | | | | | | | (4 + 4) | | | |
| 6 | 0 | 20 | 10 | 50 | W | 60 | 9 | 5 | CRPC | Lymph node |
| | | | | | | | (4 + 5) | | | |
| 7 | 85 | 10 | 0 | 275 | W | 55 | 9 | 9 | CRPC | Lymph node |
| | | | | | | | (4 + 5) | | | |
| 8 | 95 | 0 | 0 | 285 | AA | 54 | 7 | 4 | CRPC | Lymph node |
| | | | | | | | (3 + 4) | | | |
| 9 | 5 | 10 | 5 | 40 | W | 52 | 9 | 4 | CRPC | Liver |
| | | | | | | | (4 + 5) | | | |
| 10 | 10 | 10 | 0 | 50 | W | 53 | 8 | 8 | CRPC | Lymph node |
| | | | | | | | (4 + 4) | | | |
| 11 | 2 | 0 | 20 | 26 | H | 56 | 9 | 4 | CRPC | Liver |
| | | | | | | | (4 + 5) | | | |
| 12 | 10 | 20 | 0 | 70 | W | 76 | 9 | 3 | CRPC | Bone |
| | | | | | | | (4 + 5) | | | (Vertebrae) |
| 13 | 90 | 5 | 0 | 280 | W | 46 | 8 | 7 | CRPC | Lymph node |
| | | | | | | | (4 + 4) | | | |
| 14 | 95 | 0 | 0 | 285 | AA | 59 | 9 | 4 | CRPC | Lymph node |
| | | | | | | | (4 + 5) | | | |
| 15 | 70 | 20 | 0 | 250 | W | 63 | 9 | 6 | CRPC | Lymph node |
| | | | | | | | (4 + 5) | | | |
| 16 | 90 | 5 | 0 | 280 | W | 58 | 9 | 4 | CRPC | Lymph node |
| | | | | | | | (4 + 5) | | | |
| 17 | 5 | 10 | 0 | 35 | W | 49 | 8 | 9 | CRPC | Lymph node |
| | | | | | | | (4 + 4) | | | |
| 18 | 60 | 20 | 0 | 220 | W | 77.5 | 8 | 5 | CRPC | Liver |
| | | | | | | | (4 + 4) | | | |
| 19 | 5 | 20 | 10 | 65 | W | 60 | 9 | 4 | CRPC | Lymph node |
| | | | | | | | (4 + 5) | | | |
| 20 | 95 | 0 | 1 | 286 | AA | 64 | 9 | 5 | CRPC | Lymph node |
| | | | | | | | (4 + 5) | | | |
| 21 | 20 | 10 | 10 | 90 | W | 57 | 8 | 7 | CRPC | Liver |
| | | | | | | | (4 + 4) | | | |
| 22 | 0 | 0 | 2 | 2 | W | 54 | 9 | 4 | CRPC | Liver |
| | | | | | | | (4 + 5) | | | |
| 23 | 10 | 30 | 5 | 95 | AA | 59 | 9 | 9 | CRPC | Lymph node |
| | | | | | | | (4 + 5) | | | |
| 24 | 40 | 35 | 5 | 195 | W | 74 | NR | NA | CRPC | Bone |
| | | | | | | | | | | (Vertebrae) |
| 25 | 80 | 10 | 5 | 265 | AA | 57 | NR | NA | CRPC | Bone |
| | | | | | | | | | | (Vertebrae) |
| 26 | 90 | 5 | 0 | 280 | AA | 69 | NR | NA | Hormone | Bone |
| | | | | | | | | | naïve | (Vertebrae) |
| 27 | 5 | 20 | 10 | 65 | W | 73 | NR | NA | CRPC | Bone (Illium) |
| 28 | 90 | 5 | 0 | 280 | W | 56 | NR | NA | Recurrence | Bone (Illium) |
| 29 | 40 | 10 | 10 | 150 | W | 67 | NR | NA | CRPC | Bone (Illium) |
| 30 | 95 | 0 | 0 | 285 | W | 66 | NR | NA | CRPC | Bone |
| | | | | | | | | | | (Vertebrae) |
| 31 | 90 | 0 | 0 | 270 | AA | 59 | NR | NA | CRPC | Bone |
| | | | | | | | | | | (Vertebrae) |
| 32 | 0 | 0 | 5 | 5 | W | 81 | NR | NA | CRPC | bone |
| 33 | 70 | 20 | 0 | 250 | W | 75 | NR | NA | CRPC | bone |
| 34 | 10 | 10 | 10 | 60 | AA | 69 | NR | NA | NA | Bone |
| | | | | | | | | | | (Vertebrae) |

Notes:
¹Patients 1-23 from authors J. L. and E. S. A. and patients 24-34 from A. M. D.
²Race: W = White, AA = African-American, H = Hispanic
³Age at Diagnosis
⁴Gleason summary and scores (in parentheses); NR, not relevant (Gleason not scored for metastases)
⁵NA, not available.
⁶Site of bone metastases in indicated if known.

TABLE S4
List of antibodies used in this study
Primary antibodies

| | | | | Use and dilution | |
|---|---|---|---|---|---|
| Antigen | Company | Catalog # | Type | IHC/IF | Western |
| GFP | Abcam | ab13970 | Chicken pAb | 1/1000 (IF) | |
| GFP | Sigma | 11814460001 | Mouse mAb | 1/1000 (IHC) | |
| Cytokeratin 5 | Covance | PRB-160P | Rabbit pAb | 1/500 | |
| Cytokeratin 8 | Covance | MMS-162P | Mouse mAb | 1/500 | |
| Ki67 | eBiosciences | 14-5698-82 | Rat IGa2 | 1/700 | |
| AR | Abcam | ab133273 | Rabbit mAb | 1/200 | |
| MYC | Abeam | ab32072 | Rabbit mAb | 1/200 | 1/1000 |
| ATAD2 | Abcam | ab244431 | Rabbit pAb | 1/250 | 1/500 |
| β-actin | Cell Signaling | cs4970 | Rabbit mAb | | 1/20000 |

Secondary antibodies

| Antigen | Company | Catalog # | Conjugate | Use | Dilution |
|---|---|---|---|---|---|
| Goat anti-rabbit IgG | Life | A11008 | Alexa Fluor ® | IF | 1/1000 |
| | Technologies | | 488 | | |
| Goat anti-mouse IgG | Life | A11001 | Alexa Fluor ® | IF | 1/1000 |
| | Technologies | | 488 | | |
| Goat anti-rat IgG | Life | A11006 | Alexa Fluor ® | IF | 1/1000 |
| | Technologies | | 488 | | |
| Goat Anti-chicken IgG | Invitrogen | A-21437 | Alexa Fluor ® | IF | 1/1000 |
| | | | 555 | | |
| Goat anti-rabbit IgG | Invitrogen | A-21245 | Alexa Fluor ® | IF | 1/1000 |
| | | | 647 | | |
| Horse anti-rabbit IgG | Vector Labs | BA-1000 | Biotinylated | IHC | 1/300 |
| Horse anti-mouse IgG | Vector Labs | BA-2000 | Biotinylated | IHC | 1/300 |
| Horse anti-rat IgG | Vector Labs | BA-9400 | Biotinylated | IHC | 1/300 |

TABLE S5
List of primers and shRNA used in this study
Primers

| Target | Species | Forward (5′ to 3′) | Reverse (5′ to 3′) |
|---|---|---|---|
| ACTB | Human | CAACCGCGAGAAGATGACC (SEQ | AGCACAGCCTGGATAGCAAC (SEQ |
| | | ID NO: 1) | ID NO: 2) |
| ATAD2 | Human | GGGCTAGAAACATCGTTCAAAGT | GCATGGACTGGTTTACACCAC (SEQ |
| | | (SEQ ID NO: 3) | ID NO: 4) |
| AZIN1 | Human | GCCATTCTACACAGTGAAGTGC | GAACAAGCAAATCCGGTTCCA |
| | | (SEQ ID NO: 5) | (SEQ ID NO: 6) |
| CCNE2 | Human | TCAAGACGAAGTAGCCGTTTAC | TGACATCCTGGGTAGTTTTCCTC |
| | | (SEQ ID NO: 7) | (SEQ ID NO: 8) |
| ERCC6L | Human | CAAGGATGAACGGACCAGAAA | GCTTGAAAGTTGCTGCCAGTTA |
| | | (SEQ ID NO: 9) | (SEQ ID NO: 10) |
| GAPDH | Human | TTCACCACCATGGAGAAGG (SEQ | AGGGGGCAGAGATGATGAC (SEQ |
| | | ID NO: 11) | ID NO: 12) |
| LMNB1 | Human | ACATGGAAATCAGTGCTTACAGG | GGGATACTGTCACACGGGA (SEQ |
| | | (SEQ ID NO: 13) | ID NO: 14) |
| MAD2L1 | Human | GTTCTTCTCATTCGGCATCAACA | GAGTCCGTATTTCTGCACTCG (SEQ |
| | | (SEQ ID NO: 15) | ID NO: 16) |
| MCM4 | Human | CACCACACACAGTTATCCTGTT | CGAATAGGCACAGCTCGATAGAT |
| | | (SEQ ID NO: 17) | (SEQ ID NO: 18) |
| MYC | Human | AATGAAAAGGCCCCCAAGGTAGT | GTCGTTTCCGCAACAAGTCCTCTTC |
| | | TATCC (SEQ ID NO: 19) | (SEQ ID NO: 20) |
| RACGAP1 | Human | TGCACGTAATCAGGTGGATGT | TGAATCTGTCGTTCCAGCTTTT |
| | | (SEQ ID NO: 21) | (SEQ ID NO: 22) |
| RAD21 | Human | AGCCTGCACATGACGATATGG | ATGGTTGGCATTGGTTCAACG (SEQ |
| | | (SEQ ID NO: 23) | ID NO: 24) |
| RAD51API | Human | ATGACAAGCTCTACCAGAGAGAC | CACATTAGTGGTGACTGTTGGAA |
| | | (SEQ ID NO: 25) | (SEQ ID NO: 26) |
| SRPK1 | Human | ATGGAGCGGAAAGTGCTTG (SEQ | GAGCCTCGGTGCTGAGTTT (SEQ ID |
| | | ID NO: 27) | NO: 28) |
| TAF2 | Human | CGTTGATGAGTTAAAGGTCCTGA | CTCTGCCATACTTCCCTCTACA |
| | | (SEQ ID NO: 29) | (SEQ ID NO: 30) |
| TMPO | Human | CCCCTCGGTCCTGACAAAAG (SEQ | CGCTCTTCGTCACTGGAGAA (SEQ |
| | | ID NO: 31) | ID NO: 32) |
| TOP2A | Human | TGGCTGTGGTATTGTAGAAAGC | TTGGCATCATCGAGTTTGGGA (SEQ |
| | | (SEQ ID NO: 33) | ID NO: 34) |
| WDHD1 | Human | TCTACACCGTTGCATCTCACT (SEQ | CACATCTATGGCATTGTCTTGCT |
| | | ID NO: 35) | (SEQ ID NO: 36) |
| WDR12 | Human | GGTCATGCTGGAAGTGTAGATTC | CGATTTGTGGACTCCTCCATTT |
| | | (SEQ ID NO: 37) | (SEQ ID NO: 38) |
| Actb | Mouse | ATGGTGGGAATGGGTCAGAAG | CCATGTCGTCCCAGTTGGTAA (SEQ |
| | | (SEQ ID NO: 39) | ID NO: 40) |
| Atad2 | Mouse | CAGAACATTTGCACGAACAAAGT | CTGGTTCATGGTACTGTAACGAC |
| | | (SEQ ID NO: 41) | (SEQ ID NO: 42) |
| Azin1 | Mouse | ATTGACGATGCGAACTACTCCG | TTCCCAAGATCCCCCACAAAA |
| | | (SEQ ID NO: 43) | (SEQ ID NO: 44) |
| Ccne2 | Mouse | AGCCGTTTACAAGCTAAGCAA | TGGCCTGAATTATCTGGGTTTC |
| | | (SEQ ID NO: 45) | (SEQ ID NO: 46) |
| Ercc61 | Mouse | ATACATGGGTCAACGAATTTGCC | TTCTAGTGCGTTCACTCTTGC (SEQ |
| | | (SEQ ID NO: 47) | ID NO: 48) |
| Gapdh | Mouse | ACTCCACTCACGGCAAATTC (SEQ | TCTCCATGGTGGTGAAGACA (SEQ |
| | | ID NO: 49) | ID NO: 50) |
| Lmnb1 | Mouse | ATTTGGAGAATCGCTGTCAGAG | AAGCGGGTCTCATGCTTCC (SEQ ID |
| | | (SEQ ID NO: 51) | NO: 52) |
| Mad211 | Mouse | GTGGCCGAGTTTTTCTCATTTG | AGGTGAGTCCATATTTCTGCACT |
| | | (SEQ ID NO: 53) | (SEQ ID NO: 54) |
| Mcm4 | Mouse | GAGGAAAGCAGGTCGTCACC (SEQ | TGGGCATTGGCAGTAGCTC (SEQ ID |
| | | ID NO: 55) | NO: 56) |
| Myc | Mouse | CCCTATTTCATCTGCGACGAG | GAGAAGGACGTAGCGACCG (SEQ |
| | | (SEQ ID NO: 57) | ID NO: 58) |
| Racgap1 | Mouse | CAGTGACTCCGCTCTGAACAG | TGAGACGAAGTCGTGCAAGC (SEQ |
| | | (SEQ ID NO: 59) | ID NO: 60) |
| Rad21 | Mouse | GGAGTAGTCCGCATCTATCACA | GGCCGAAACGCCATCTTTATT (SEQ |
| | | (SEQ ID NO: 61) | ID NO: 62) |
| Rad51ap1 | Mouse | CCTTCTGAGGCCACTAGGAAA | TAACTGGCGCTAATCGGGAGA |
| | | (SEQ ID NO: 63) | (SEQ ID NO: 64) |
| Srpk1 | Mouse | AGGCCCGAAAGAAAAGGACC | TGCTCTGGGATGTCGCTCT (SEQ ID |
| | | (SEQ ID NO: 65) | NO: 66) |
| Taf2 | Mouse | GGCCTTGGAAAAATTCCCCAC | GAAGCACGCTGACATCCTGA (SEQ |
| | | (SEQ ID NO: 67) | ID NO: 68) |
| Tmpo | Mouse | GAAGAACTTCTGGATCAGCTTGT | CCCTCAGCTTCAACAGCTTC (SEQ |
| | | (SEQ ID NO: 69) | ID NO: 70) |
| Top2a | Mouse | AACAAAGGGACCCAAAAATGTCT | TGTGTTCAACAACAGGGATTCC |
| | | (SEQ ID NO: 71) | (SEQ ID NO: 72) |
| Wdhd1 | Mouse | AGCCCATGAGGTACGGACATA | ACTGCCACAAGTCACAATATAGC |
| | | (SEQ ID NO: 73) | (SEQ ID NO: 74) |
| Wdr12 | Mouse | CTGAAGTTGCGGACCTTAGTAAC | AAATCAAACTCGACATGCTTGTG |
| | | (SEQ ID NO: 75) | (SEQ ID NO: 76) |

shRNA

| Target | Company | Catalog #/CloneID | Oligo Sequence |
|---|---|---|---|
| Control | Sigma | SHC002 | CAACAAGATGAAGAGCACCAA |
| | | | (SEQ ID NO: 77) |
| shMyc #1 | Dharmacon | TRCN0000042513 | CGAGAACAGTTGAAACACAAA |
| (mouse) | | | (SEQ ID NO: 78) |
| shMyc #2 | Dharmacon | TRCNO000042515 | CGACGAGGAAGAGAATTTCTA |
| (mouse) | | | (SEQ ID NO: 79) |
| shMYC #1 | Dharmacon | TRCN0000039640 | CAGTTGAAACACAAACTTGAA |
| (human) | | | (SEQ ID NO: 80) |
| shMYC #2 | Dharmacon | TRCN0000039642 | CCTGAGACAGATCAGCAACAA |
| (human) | | | (SEQ ID NO: 81) |

The following are four Supplementary Datasets used herein:

Supplementary Dataset 1: Differentially expressed genes from bulk RNA sequencing of NPK-CAG^{YFP} prostate tumors and metastases

Supplementary Dataset 2: Differentially expressed genes from single-cell sequencing of NPK-CAG^{YFP} prostate tumors and bone metastases

Supplementary Dataset 3: Biological pathway analysis from NPK-CAG^{YFP} prostate tumors and metastases

Supplementary Dataset 4: Single nucleotide variants based on whole exome sequencing of NPK-CAG^{YFP} prostate tumors and metastases.

All four Datasets are reproduced below:

Lengthy table referenced here
US20220307089A1-20220929-T00001
Please refer to the end of the specification for access instructions.

Lengthy table referenced here
US20220307089A1-20220929-T00002
Please refer to the end of the specification for access instructions.

Lengthy table referenced here
US20220307089A1-20220929-T00003
Please refer to the end of the specification for access instructions.

Lengthy table referenced here
US20220307089A1-20220929-T00004
Please refer to the end of the specification for access instructions.

Lengthy table referenced here
US20220307089A1-20220929-T00005
Please refer to the end of the specification for access instructions.

Lengthy table referenced here
US20220307089A1-20220929-T00006
Please refer to the end of the specification for access instructions.

Lengthy table referenced here
US20220307089A1-20220929-T00007
Please refer to the end of the specification for access instructions.

Lengthy table referenced here
US20220307089A1-20220929-T00008
Please refer to the end of the specification for access instructions.

Lengthy table referenced here
US20220307089A1-20220929-T00009
Please refer to the end of the specification for access instructions.

Lengthy table referenced here
US20220307089A1-20220929-T00010
Please refer to the end of the specification for access instructions.

Lengthy table referenced here
US20220307089A1-20220929-T00011
Please refer to the end of the specification for access instructions.

Lengthy table referenced here
US20220307089A1-20220929-T00012
Please refer to the end of the specification for access instructions.

Until now it has proven challenging to model high-efficiency bone metastasis in the context of the native tumor microenvironment during cancer evolution in the whole organism. Our description of NPK^{EYFP} mice overcome this limitation since these mice model lethal prostate cancer with highly penetrant bone metastasis. Indeed, the present analyses of NPK^{EYFP} mice have enabled detailed biological and molecular characterization of bone metastases as arise de novo during tumor progression in vivo in androgen-intact and androgen-deprived contexts.

Although bone metastases are only discernable in highly metastatic NPK^{EYFP} mice, longitudinal analysis combined with phylogenetic analysis reveal that they originate early in disease progression from an early sub-clone of the primary tumor. It can be inferred herein that bone metastases in NPK^{EYFP} mice are seeded early, but take longer to cultivate compared with metastases to soft tissues. This parallels the scenario in human patients, wherein bone metastases are more prevalent than metastases to visceral tissues, but the latter are associated with worse clinical outcome. Since DTCs occur in bones of NPK mice early during prostate cancer progression, analyses of circulating tumor cells in NPK^{EYFP} mice can help to identify and molecularly characterize bone metastases early in tumor progression.

The current embodiments recognize the MYC activation in mCRPC and metastasis, and the importance of MYC in prostate cancer, notably, in certain embodiments the co-activation of MYC and RAS as a driver of prostate cancer metastasis.

In certain embodiments, the META-55 and META-16 gene signatures, associated with adverse outcome for metastasis in patients with localized prostate cancer and adverse treatment response in patients with advanced disease, can augment other prognostic signatures, such as Decipher GX, which is associated with risk of metastasis, and Prolaris CCP score, which is associated with prostate cancer specific survival.

The present disclosure has demonstrated that bone metastases can have distinct sub-clonal origin and transcriptomic profiles. The present development of a GEMM of bone metastasis has uncovered new mechanisms relevant for human prostate cancer metastasis that are likely to provide new opportunities for improved detection and treatment of this currently intractable disease.

Although the present technology has been described in relation to embodiments thereof, these embodiments and examples are merely exemplary and not intended to be limiting. Any reference to a particular Figure indicates only a particular embodiment, and does not limit all embodiments contemplated herein solely to what is depicted in the Figure mentioned. Many other variations and modifications and other uses will become apparent to those skilled in the art. The present technology should, therefore, not be limited by the specific disclosure herein, and can be embodied in other forms not explicitly described here, without departing from the spirit thereof.

LENGTHY TABLES
The patent application contains a lengthy table section. A copy of the table is available in electronic form from the USPTO web site (https://seqdata.uspto.gov/?pageRequest=docDetail&DocID=US20220307089A1). An electronic copy of the table will also be available from the USPTO upon request and payment of the fee set forth in 37 CFR 1.19(b)(3).

What is claimed is:

1. A method for diagnosing metastasis in a subject having cancer, or for assessing risk of metastasis in a subject having cancer, the method comprising:
(a) obtaining a sample from the subject;
(b) determining in the sample an expression level of one or more of genes ATAD2, AZIN1, CCNE2, ERCC6L, LMNB1, MAD2L1, MCM4, RACGAP1, RAD21, RAD51AP1, SRPK1, TAF2, TMPO, TOP2A, WDHD1, WDR12, CSE1L, LRPPRC, DHX9, UBA2, G3BP1, HEATR1, MRPL3, DDX21, ARL6IP1, CHD1L, PAXIP1, ACTL6A, RRP15, NUP107, CENPN, DBF4, SLC25A5, RAN, CCT5, HNRNPAB, AFG3L2, TMEM97, WDR3, CDC23, NFYA, MSH2, MAPKAPK5, POLR3B, GART, C1QBP, ECT2, DSCC1, TRMT12, SLBP, UNG, TTK, KIF20A, TRIP13, or TIMELESS;
(c) comparing the expression level obtained in step (b) with a reference level or with an expression level of the one or more genes in a control sample; and
(d) diagnosing that the subject has metastasis or an increased risk of metastasis, if the expression level of at least one gene obtained in step (b) increases by at least 10% compared to the reference level or its expression level in the control sample.

2. A method for treating a subject with metastatic cancer or an increased risk of cancer metastasis, the method comprising:
(a) obtaining a sample from the subject;
(b) determining in the sample an expression level of one or more of genes ATAD2, AZIN1, CCNE2, ERCC6L, LMNB1, MAD2L1, MCM4, RACGAP1, RAD21, RAD51AP1, SRPK1, TAF2, TMPO, TOP2A, WDHD1, WDR12, CSE1L, LRPPRC, DHX9, UBA2, G3BP1, HEATR1, MRPL3, DDX21, ARL6IP1, CHD1L, PAXIP1, ACTL6A, RRP15, NUP107, CENPN, DBF4, SLC25A5, RAN, CCT5, HNRNPAB, AFG3L2, TMEM97, WDR3, CDC23, NFYA, MSH2, MAPKAPK5, POLR3B, GART, C1QBP, ECT2, DSCC1, TRMT12, SLBP, UNG, TTK, KIF20A, TRIP13, or TIMELESS;
(c) comparing the expression level obtained in step (b) with a reference level or with an expression level of the one or more genes in a control sample; and
(d) treating the subject for metastatic cancer or an increased risk of cancer metastasis, if the expression level of at least one gene obtained in step (b) increases by at least 10% compared to the reference level or its expression level in the control sample.

3. The method of claim 1 or 2, wherein the cancer is prostate cancer.

4. The method of claim 1 or 2, wherein the metastasis is bone metastasis.

5. The method of claim 4, wherein the bone metastasis is osteolytic metastasis.

6. The method of claim 1 or 2, wherein the increase in the expression level of the one or more genes is at least 15%.

7. The method of claim 1 or 2, wherein the increase in the expression level of the one or more genes is at least 20%.

8. The method of claim 1 or 2, wherein the increase in the expression level of the one or more genes is at least 30%.

9. The method of claim 1 or 2, wherein the increase in the expression level of the one or more genes is at least 50%.

10. The method of claim 1 or 2, wherein the increase in the expression level of the one or more genes is about 20% to about 90%.

11. The method of claim 1 or 2, wherein the sample is a plasma, serum or blood sample.

12. The method of claim 1 or 2, wherein the sample is a prostate tumor sample.

13. The method of claim 1 or 2, wherein the control sample is from a healthy subject or a plurality of healthy subjects.

14. The method of claim 1 or 2, wherein the control sample is from a subject having a metastasis-free cancer.

15. The method of claim 1 or 2, wherein the subject is human.

16. The method of claim 1 or 2, wherein the expression level of the one or more genes is determined by assaying an mRNA level or a protein level.

17. The method of claim 1 or 2, wherein the expression level of the one or more genes is determined by polymerase chain reaction (PCR), RNA sequencing (RNA-seq), or nCounter technology.

18. A kit comprising:
(a) means for quantifying an expression level of one or more genes selected from the group consisting of ATAD2, AZIN1, CCNE2, ERCC6L, LMNB1, MAD2L1, MCM4, RACGAP1, RAD21, RAD51AP1, SRPK1, TAF2, TMPO, TOP2A, WDHD1, WDR12, CSE1L, LRPPRC, DHX9, UBA2, G3BP1, HEATR1, MRPL3, DDX21, ARL6IP1, CHD1L, PAXIP1, ACTL6A, RRP15, NUP107, CENPN, DBF4, SLC25A5, RAN, CCT5, HNRNPAB, AFG3L2, TMEM97, WDR3, CDC23, NFYA, MSH2, MAPKAPK5, POLR3B, GART, C1QBP, ECT2, DSCC1, TRMT12, SLBP, UNG, TTK, KIF20A, TRIP13, and TIMELESS, in a sample from a subject;
b) means for comparing the expression level with a reference level or with an expression level of the one or more genes in a control sample; and, optionally,
c) means for determining a therapy for treating the subject.


SEQUENCE LISTING (81 sequences)

SEQ ID NO: 1 - DNA, 19 bp, Homo sapiens
caaccgcgagaagatgacc

SEQ ID NO: 2 - DNA, 20 bp, Homo sapiens
agcacagcctggatagcaac

SEQ ID NO: 3 - DNA, 23 bp, Homo sapiens
gggctagaaacatcgttcaaagt

SEQ ID NO: 4 - DNA, 21 bp, Homo sapiens
gcatggactggtttacaccac

SEQ ID NO: 5 - DNA, 22 bp, Homo sapiens
gccattctacacagtgaagtgc

SEQ ID NO: 6 - DNA, 21 bp, Homo sapiens
gaacaagcaaatccggttcca

SEQ ID NO: 7 - DNA, 22 bp, Homo sapiens
tcaagacgaagtagccgtttac

SEQ ID NO: 8 - DNA, 23 bp, Homo sapiens
tgacatcctgggtagttttcctc

SEQ ID NO: 9 - DNA, 21 bp, Homo sapiens
caaggatgaacggaccagaaa

SEQ ID NO: 10 - DNA, 22 bp, Homo sapiens
gcttgaaagttgctgccagtta

SEQ ID NO: 11 - DNA, 19 bp, Homo sapiens
ttcaccaccatggagaagg

SEQ ID NO: 12 - DNA, 19 bp, Homo sapiens
agggggcagagatgatgac

SEQ ID NO: 13 - DNA, 23 bp, Homo sapiens
acatggaaatcagtgcttacagg

SEQ ID NO: 14 - DNA, 19 bp, Homo sapiens
gggatactgtcacacggga

SEQ ID NO: 15 - DNA, 23 bp, Homo sapiens
gttcttctcattcggcatcaaca

SEQ ID NO: 16 - DNA, 21 bp, Homo sapiens
gagtccgtatttctgcactcg

SEQ ID NO: 17 - DNA, 22 bp, Homo sapiens
caccacacacagttatcctgtt

SEQ ID NO: 18 - DNA, 23 bp, Homo sapiens
cgaataggcacagctcgatagat

SEQ ID NO: 19 - DNA, 28 bp, Homo sapiens
aatgaaaaggcccccaaggtagttatcc

SEQ ID NO: 20 - DNA, 25 bp, Homo sapiens
gtcgtttccgcaacaagtcctcttc

SEQ ID NO: 21 - DNA, 21 bp, Homo sapiens
tgcacgtaatcaggtggatgt

SEQ ID NO: 22 - DNA, 22 bp, Homo sapiens
tgaatctgtcgttccagctttt

SEQ ID NO: 23 - DNA, 21 bp, Homo sapiens
agcctgcacatgacgatatgg

SEQ ID NO: 24 - DNA, 21 bp, Homo sapiens
atggttggcattggttcaacg

SEQ ID NO: 25 - DNA, 23 bp, Homo sapiens
atgacaagctctaccagagagac

SEQ ID NO: 26 - DNA, 23 bp, Homo sapiens
cacattagtggtgactgttggaa

SEQ ID NO: 27 - DNA, 19 bp, Homo sapiens
atggagcggaaagtgcttg

SEQ ID NO: 28 - DNA, 19 bp, Homo sapiens
gagcctcggtgctgagttt

SEQ ID NO: 29 - DNA, 23 bp, Homo sapiens
cgttgatgagttaaaggtcctga

SEQ ID NO: 30 - DNA, 22 bp, Homo sapiens
ctctgccatacttccctctaca

SEQ ID NO: 31 - DNA, 20 bp, Homo sapiens
cccctcggtcctgacaaaag

SEQ ID NO: 32 - DNA, 20 bp, Homo sapiens
cgctcttcgtcactggagaa

SEQ ID NO: 33 - DNA, 22 bp, Homo sapiens
tggctgtggtattgtagaaagc

SEQ ID NO: 34 - DNA, 21 bp, Homo sapiens
ttggcatcatcgagtttggga

SEQ ID NO: 35 - DNA, 21 bp, Homo sapiens
tctacaccgttgcatctcact

SEQ ID NO: 36 - DNA, 23 bp, Homo sapiens
cacatctatggcattgtcttgct

SEQ ID NO: 37 - DNA, 23 bp, Homo sapiens
ggtcatgctggaagtgtagattc

SEQ ID NO: 38 - DNA, 22 bp, Homo sapiens
cgatttgtggactcctccattt

SEQ ID NO: 39 - DNA, 21 bp, Mus musculus
atggtgggaatgggtcagaag

SEQ ID NO: 40 - DNA, 21 bp, Mus musculus
ccatgtcgtcccagttggtaa

SEQ ID NO: 41 - DNA, 23 bp, Mus musculus
cagaacatttgcacgaacaaagt

SEQ ID NO: 42 - DNA, 23 bp, Mus musculus
ctggttcatggtactgtaacgac

SEQ ID NO: 43 - DNA, 22 bp, Mus musculus
attgacgatgcgaactactccg

SEQ ID NO: 44 - DNA, 21 bp, Mus musculus
ttcccaagatcccccacaaaa

SEQ ID NO: 45 - DNA, 21 bp, Mus musculus
agccgtttacaagctaagcaa

SEQ ID NO: 46 - DNA, 22 bp, Mus musculus
tggcctgaattatctgggtttc

SEQ ID NO: 47 - DNA, 23 bp, Mus musculus
atacatgggtcaacgaatttgcc

SEQ ID NO: 48 - DNA, 21 bp, Mus musculus
ttctagtgcgttcactcttgc

SEQ ID NO: 49 - DNA, 20 bp, Mus musculus
actccactcacggcaaattc

SEQ ID NO: 50 - DNA, 20 bp, Mus musculus
tctccatggtggtgaagaca

SEQ ID NO: 51 - DNA, 22 bp, Mus musculus
atttggagaatcgctgtcagag

SEQ ID NO: 52 - DNA, 19 bp, Mus musculus
aagcgggtctcatgcttcc

SEQ ID NO: 53 - DNA, 22 bp, Mus musculus
gtggccgagtttttctcatttg

SEQ ID NO: 54 - DNA, 23 bp, Mus musculus
aggtgagtccatatttctgcact

SEQ ID NO: 55 - DNA, 20 bp, Mus musculus
gaggaaagcaggtcgtcacc

SEQ ID NO: 56 - DNA, 19 bp, Mus musculus
tgggcattggcagtagctc

SEQ ID NO: 57 - DNA, 21 bp, Mus musculus
ccctatttcatctgcgacgag

SEQ ID NO: 58 - DNA, 19 bp, Mus musculus
gagaaggacgtagcgaccg

SEQ ID NO: 59 - DNA, 21 bp, Mus musculus
cagtgactccgctctgaacag

SEQ ID NO: 60 - DNA, 20 bp, Mus musculus
tgagacgaagtcgtgcaagc

SEQ ID NO: 61 - DNA, 22 bp, Mus musculus
ggagtagtccgcatctatcaca

SEQ ID NO: 62 - DNA, 21 bp, Mus musculus
ggccgaaacgccatctttatt

SEQ ID NO: 63 - DNA, 21 bp, Mus musculus
ccttctgaggccactaggaaa

SEQ ID NO: 64 - DNA, 21 bp, Mus musculus
taactggcgctaatcgggaga

SEQ ID NO: 65 - DNA, 20 bp, Mus musculus
aggcccgaaagaaaaggacc

SEQ ID NO: 66 - DNA, 19 bp, Mus musculus
tgctctgggatgtcgctct

SEQ ID NO: 67 - DNA, 21 bp, Mus musculus
ggccttggaaaaattccccac

SEQ ID NO: 68 - DNA, 20 bp, Mus musculus
gaagcacgctgacatcctga

SEQ ID NO: 69 - DNA, 23 bp, Mus musculus
gaagaacttctggatcagcttgt

SEQ ID NO: 70 - DNA, 20 bp, Mus musculus
ccctcagcttcaacagcttc

SEQ ID NO: 71 - DNA, 23 bp, Mus musculus
aacaaagggacccaaaaatgtct

SEQ ID NO: 72 - DNA, 22 bp, Mus musculus
tgtgttcaacaacagggattcc

SEQ ID NO: 73 - DNA, 21 bp, Mus musculus
agcccatgaggtacggacata

SEQ ID NO: 74 - DNA, 23 bp, Mus musculus
actgccacaagtcacaatatagc

SEQ ID NO: 75 - DNA, 23 bp, Mus musculus
ctgaagttgcggaccttagtaac

SEQ ID NO: 76 - DNA, 23 bp, Mus musculus
aaatcaaactcgacatgcttgtg

SEQ ID NO: 77 - DNA, 21 bp, Unknown
  Control Sequence
caacaagatgaagagcaccaa

SEQ ID NO: 78 - DNA, 21 bp, Mus musculus
cgagaacagttgaaacacaaa

SEQ ID NO: 79 - DNA, 21 bp, Mus musculus
cgacgaggaagagaatttcta

SEQ ID NO: 80 - DNA, 21 bp, Homo sapiens
cagttgaaacacaaacttgaa

SEQ ID NO: 81 - DNA, 21 bp, Homo sapiens
cctgagacagatcagcaacaa